(19) United States
(12) Patent Application Publication
(10) Pub. No.: US 2025/0135029 A1
(43) Pub. Date: May 1, 2025

(54) REGULATORY ELEMENT FOR CELL TYPE SPECIFIC EXPRESSION OF GENES IN SPINAL MOTOR NEURONS

(71) Applicant: The Trustees of Columbia University in the City of New York, New York, NY (US)

(72) Inventors: Hynek WICHTERLE, New York, NY (US); Tulsi PATEL, New York, NY (US)

(21) Appl. No.: 19/013,368

(22) Filed: Jan. 8, 2025

Related U.S. Application Data
(63) Continuation of application No. PCT/US23/69780, filed on Jul. 7, 2023.
(60) Provisional application No. 63/359,527, filed on Jul. 8, 2022.

(51) Int. Cl. A61K 48/00; C12N 15/86

(52) U.S. Cl. CPC A61K 48/0041; C12N 15/86; C12N 2750/14143; C12N 2830/008

(57) ABSTRACT

The subject matter described here relates to an enhancer specific to a subset of cholinergic neurons, that can control gene expression in a highly neuron-specific manner, wherein the enhancer is compatible with AAVs packaging and can induce gene expression or knockdown in cholinergic neurons in both post-natal and adult subjects.

This application is a continuation of International Patent Application No. PCT/US2023/069780, filed on Jul. 7, 2023 which claims the benefit of and priority to U.S. Provisional Patent Application No. 63/359,527, filed Jul. 8, 2022, the contents of each of which are incorporated herein by reference in their entireties.

All patents, patent applications and publications cited herein are hereby incorporated by reference in their entirety.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### GOVERNMENT SUPPORT

This invention was made with government support under grant nos. NS116141, NS105372, NS121136, and NS109217 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 6, 2025, is named 0019240_01268US2_SL.xml and is 208,484 bytes in size.

### BACKGROUND OF THE INVENTION

Cholinergic neurons, including motor neurons and basal forebrain cholinergic neurons, are implicated in various neurodegenerative diseases including Amyotrophic lateral sclerosis (ALS) and Alzheimer's disease.

ALS is a fatal motor neuron disease affecting about 4.42 per 100,000 people. It is characterized by motor neuron degeneration resulting in paralysis and death. Currently, only two drugs to treat ALS are improved by the United States Food and Drug Administration edaravone and riluzole—and their effects on improving life expectancy is limited. While focusing on motor neurons is essential for future therapeutic treatments, targeting their widespread distribution across developmental stages remains a limiting factor for potential gene therapy.

Basal forebrain cholinergic neurons are affected in Alzheimer's disease, the most common form of dementia and estimated to affect 5.8 million Americans aged 65 years or older as of 2020. Alzheimer's disease is fatal, and it currently has no cure.

There is a growing interest in gene therapy for the treatment of neurodegenerative diseases. However, broad expression of transgenes or knockdown construct could potentially have deleterious side effects. Therefore, having the ability to control gene expression in a highly neuron-specific and temporally-controlled manner will be beneficial.

### SUMMARY OF THE INVENTION

Described herein is an enhancer specific to a subset of cholinergic neurons, including motor neurons, that can specifically control gene expression, and methods of implementing the technology.

In certain aspects, described herein is a nucleic acid comprising an enhancer sequence comprising a nucleotide sequence at least 70% identical to SEQ ID NO: 1 and a nucleotide sequence encoding a polypeptide of interest or an oligonucleotide of interest, wherein the nucleotide sequence encoding the polypeptide of interest or oligonucleotide of interest is positioned 3′ to the enhancer sequence.

In some embodiments, the enhancer sequence drives expression of the polypeptide of interest or oligonucleotide of interest in a cholinergic neuron. In certain embodiments, the cholinergic neuron is a motor neuron. In some embodiments, the motor neuron is a lower motor neuron. In certain embodiments, the cholinergic neuron is a basal forebrain cholinergic neuron.

In some embodiments, the nucleic acid comprises a nucleotide sequence encoding a polypeptide of interest. In certain embodiments, the polypeptide of interest is a neurotrophic factor or a neuroprotective factor. In some embodiments, the polypeptide of interest is a prophylactic or therapeutic polypeptide. In certain embodiments, the prophylactic or therapeutic polypeptide is brain-derived neurotrophic factor (BDNF), glial cell line derived neurotrophic factor (GDNF), insulin-like growth factor 1 (IGF-1), and vascular endothelial growth factors (VEGF). In some embodiments, the polypeptide of interest is a Cre recombinase.

In certain embodiments, the nucleotide sequence encodes an oligonucleotide of interest. In some embodiments, the oligonucleotide of interest is a non-coding RNA. In certain embodiments, the non-coding RNA is an inhibitory RNA. In some embodiments, the inhibitory is one or more of a miRNA, a siRNA, a shRNA, or a piRNA. In certain embodiments, the inhibitory RNA mediates silencing of a target gene. In some embodiments, the target gene is SOD1, C9orf72, ATXN2, FUS, or L3MRTL1. In certain embodiments, the non-coding RNA is a CRISPR/Cas9 guide RNA or single guide RNA.

In certain embodiments, the nucleic acid further comprises a nucleotide sequence encoding a selectable marker gene. In some embodiments, the selectable marker gene is an antibiotic resistance gene.

In certain embodiments, the nucleic acid further comprises a nucleotide sequence encoding a promoter sequence positioned between the enhancer sequence and the nucleotide sequence encoding the polypeptide of interest or an oligonucleotide of interest. In some embodiments, the nucleic acid further comprises a nucleotide sequence of an intron sequence positioned between the enhancer sequence and the nucleotide sequence encoding the polypeptide of interest or an oligonucleotide of interest. In certain embodiments, the nucleic acid comprises a nucleotide sequence encoding a polypeptide of interest and further comprises a nucleotide sequence of a polyA sequence positioned 3′ to the nucleotide sequence encoding the polypeptide of interest, optionally wherein the polyA sequence is an SV40 poly A sequence.

In some embodiments, the enhancer sequence is at least 80%, at least 90%, at least 95%, at least 97%, at least 98%, or at least 99% identical to SEQ ID NO: 1. In certain embodiments, the enhancer sequence comprises SEQ ID NO: 1. In some embodiments, the enhancer sequence consists of SEQ ID NO: 1.

In certain aspects, described herein is an expression cassette comprising any of the nucleic acids as described herein.

In certain aspects, described herein is a vector comprising any of the nucleic acids as described herein. In some embodiments, the vector is a viral vector. In certain embodiments, the vector is an AAV vector. In some embodiments, the nucleic acid is positioned between first inverted terminal repeat (ITR) of the AAV vector and a second inverted terminal repeat (ITR) of the AAV vector, and in some embodiments, the first and second ITRs are AAV2 ITR. In certain embodiments, the vector is circular, and in some embodiments, the vector is linearized.

In certain aspects, described herein is an AAV particle comprising any of the nucleic acids, any of the expression vectors, or any of the vectors as described herein. In some embodiments, the AAV particle is an AAV2 particle.

In certain aspects, described herein is a cell comprising any of the nucleic acids, any of the expression vectors, or any of the vectors as described herein. In some embodiments, the cell further comprises an AAV Rep gene and an AAV Cap gene, and in certain embodiments, the AAV Rep gene and/or AAV Cap gene are AAV2 genes. In some embodiments, the cell further comprises a nucleic acid comprising a nucleotide sequence encoding one or more AAV helper genes.

In certain aspects, described herein is a method of studying cholinergic activity in the central nervous system or motor neuron activity in the peripheral nervous system comprising administering the any of the AAV particles as described herein to a subject.

In certain aspects, described herein is a method of treating a neurologic disorder in a subject in need thereof comprising administering a therapeutically effective amount of any of the AAV particles as described herein to the subject. In some embodiments, the neurologic disorder is associated with a defect in cholinergic neurons. In certain embodiments, the neurologic disorder is Amyotrophic Lateral Sclerosis, Alzheimer's disease, Parkinson's disease, Myasthenia gravis, Huntington's chorea, Spinal Muscular Atrophy, Kennedy Disease, Progressive Muscular Atrophy, and Monomelic Amyotrophy. In some embodiments, the neurologic disorder is Alzheimer's disease. In certain embodiments, the neurologic disorder is Amyotrophic Lateral Sclerosis.

In certain aspects, described herein is a method of gene editing cholinergic neurons comprising administering any of the AAV particles as described herein to a subject. In some embodiments, the cholinergic neuron is a motor neuron. In certain embodiments, the motor neuron is a lower motor neuron. In some embodiments, the cholinergic neuron is a basal forebrain cholinergic neuron.

In certain embodiments, the AAV particle as described herein is administered via intravenous, intracerebroventricular, intrathecal, intraparenchymal, intramuscular, or intraperitoneal injection. In some embodiments, the subject is a mammal. In certain embodiments, the subject is a mouse or a rat. In some embodiments, the subject is a human. In certain embodiments, the polypeptide of interest or the oligonucleotide of interest is expressed for at least 4, at least 21, or at least 72 days post-administration.

In certain aspects, described herein is a pharmaceutical composition comprising any of the AAV particles as described herein.

In certain aspects, described herein is a nucleic acid comprising a nucleotide sequence at least 70% identical to SEQ ID NO: 1. In some embodiments, the nucleotide sequence is at least 80%, at least 90%, at least 95%, at least 97%, at least 98%, or at least 99% identical to SEQ ID NO: 1. In certain embodiments, the nucleotide sequence comprises SEQ ID NO: 1. In some embodiments, the nucleotide sequence consists of SEQ ID NO: 1.

These and other embodiments of the invention are further described in the following sections of the application, including the Detailed Description, Examples, Claims, and Drawings.

### BRIEF DESCRIPTION OF FIGURES

The patent or application file contains at least one drawing originally in color. To conform to the requirements for PCT patent applications, many of the figures presented herein are black and white representations of images originally created in color.

FIG. 1 shows choline acetyltransferase (ChAT) in situ labeling of spinal cord motor neurons (Image Credit: Allen Brain Atlas).

FIGS. 2A and B show functional validation of the enhancer-based AAV virus to drive the expression of proteins of interest in spinal motor neurons. FIG. 2A shows stages of injection of enhancer-based AAV virus into mice at PO and stages of analysis. FIG. 2B shows images of whole spinal cords or region 1 which contains spinal motor neurons at P4, P21 and P72. mCherry from AAV is shown in red, ChAT protein is shown in green. Motor neurons are labeled with ChAT protein.

FIGS. 3A and B show functional validation of the enhancer-based AAV virus to drive the expression of either Is11 or Lhx3 in spinal motor neurons. FIG. 3A shows ChAT in situ labeling of spinal cord motor neurons with regions 2 highlighted. FIG. 3B shows images of spinal cord region 2 as labeled in 3A. AAV virus drives the expression of either Is11 or Lhx3, two transcription factors. Both transcription factors are easily detected in motor neurons of injected mice 20 days after injection. Is11 or Lhx3 from AAV are shown in green, ChAT protein is shown in red.

FIGS. 4A and B show functional validation of the enhancer-based AAV virus to drive the expression of proteins of interest in spinal motor neurons of juvenile mice. FIG. 4A shows ChAT in situ labeling of spinal cord motor neurons with regions 1 highlighted. FIG. 4B shows images of region 1 which contains spinal motor neurons from three weeks old mice injected with an AAV virus that drives expression of fluorescent protein mCherry. Spinal cord sections are imaged 2.5 weeks after injection and detected mCherry in motor neurons. mCherry from AAV is shown in red, and ChAT protein is shown in green. Motor neurons are labeled with ChAT protein.

FIG. 5 provides the sequence for the enhancer described herein (SEQ ID NO:1).

FIG. 6 provides the sequence for plasmid pTP-AAV-ChATen-mCherry (SEQ ID NO: 10).

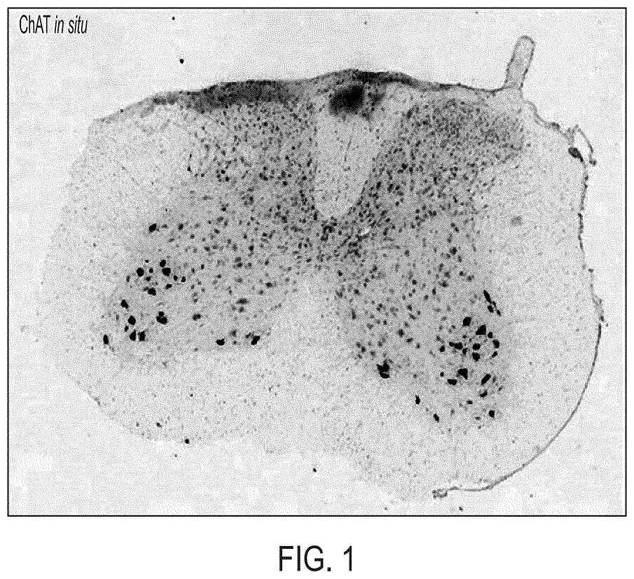

### DETAILED DESCRIPTION

In certain aspects, described herein is an enhancer specific to a subset of cholinergic neurons that can control gene expression in a highly neuron-specific and temporally-controlled manner. In certain embodiments, the enhancer described herein is smaller than 9 kb, and in some embodiments the enhancer is about 1 kb. In some embodiments, the enhancer is compatible with AAV packaging and can induce gene expression or knockdown in cholinergic neurons at different developmental stages. In some embodiments, the different developmental stages are between the early postnatal stage and adulthood.

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “subject” refers to a vertebrate animal. In some embodiments, the subject is a mammal or a mammalian species. In some embodiments, the subject is a human. In some embodiments, the subject is a healthy human adult. In other embodiments, the subject is a non-human vertebrate animal, including, without limitation, non-human primates, laboratory animals, livestock, racehorses, domesticated animals, and non-domesticated animals. In some embodiments, the term “human subjects” means a population of healthy human adults.

The term “mammal” includes, but is not limited to, a human, mouse, rat, guinea pig, dog, cat, horse, cow, pig, or non-human primate, such as a monkey, chimpanzee, baboon, or rhesus. In some embodiments, the mammal is a human.

### Nucleic Acids

In certain aspects, described herein is a nucleic acid comprising: an enhancer sequence comprising a nucleotide sequence at least 70% identical to SEQ ID NO: 1; and a nucleotide sequence encoding a polypeptide of interest or an oligonucleotide of interest, wherein the nucleotide encoding the polypeptide of interest or oligonucleotide of interest is positioned 3′ to the enhancer sequence.

In certain aspects the invention provides an expression cassette comprising a nucleic acid comprising: an enhancer sequence comprising a nucleotide sequence at least 70% identical to SEQ ID NO: 1; and a nucleotide sequence encoding a polypeptide of interest or an oligonucleotide of interest, wherein the nucleotide encoding the polypeptide of interest or oligonucleotide of interest is positioned 3′ to the enhancer sequence.

In certain aspects, the invention is directed to a nucleic acid sequence as provided in SEQ ID NO: 1.

In certain aspects, the invention is directed to nucleic acid sequence variants of SEQ ID NO: 1. Variants of SEQ ID NO: 1 include, but are not limited to, nucleic acid sequences having at least from about 50% to about 55% identity to that of SEQ ID NO: 1. Variants of SEQ ID NO: 1 include, but are not limited to, nucleic acid sequences having at least from about 55.1% to about 60% identity to that of SEQ ID NO: 1. Variants of SEQ ID NO: 1 include, but are not limited to, nucleic acid sequences having at least from about 60.1% to about 65% identity to that of SEQ ID NO: 1. Variants of SEQ ID NO: 1 include, but are not limited to, nucleic acid sequences having at least from about 65.1% to about 70% identity to that of SEQ ID NO: 1. Variants of SEQ ID NO: 1 include, but are not limited to, nucleic acid sequences having at least from about 70.1% to about 75% identity to that of SEQ ID NO: 1. Variants of SEQ ID NO: 1 include, but are not limited to, nucleic acid sequences having at least from about 75.1% to about 80% identity to that of SEQ ID NO: 1. Variants of SEQ ID NO: 1 include, but are not limited to, nucleic acid sequences having at least from about 80.1% to about 85% identity to that of SEQ ID NO: 1. Variants of SEQ ID NO: 1 include, but are not limited to, nucleic acid sequences having at least from about 85.1% to about 90% identity to that of SEQ ID NO: 1. Variants of SEQ ID NO: 1, but are not limited to, nucleic acid sequences having at least from about 90.1% to about 95% identity to that of SEQ ID NO: 1. Variants of SEQ ID NO: 1 include, but are not limited to, nucleic acid sequences having at least from about 95.1% to about 97% identity to that of SEQ ID NO: 1. Variants of SEQ ID NO: 1 include, but are not limited to, nucleic acid sequences having at least from about 97.1% to about 99% identity to that of SEQ ID NO: 1.

Programs and algorithms for sequence alignment and comparison of % identity and/or homology between nucleic acid sequences, or polypeptides, are well known in the art, and include BLAST, SIM alignment tool, and so forth.

In some embodiments, the invention is directed to a nucleic acid sequence comprising from about 10 to about 50 consecutive nucleotides from SEQ ID NO: 1.

In some embodiments, the invention is directed to an isolated nucleic acid sequence comprising from about 10 to about 100 consecutive nucleotides from any one of SEQ ID NO: 1 or a sequence complementary to SEQ ID NO: 1. In some embodiments, the invention is directed to an isolated nucleic acid sequence comprising from about 10 to about 200 consecutive nucleotides from any one of SEQ ID NO: 1 or a sequence complementary to SEQ ID NO: 1. In some embodiments, the invention is directed to an isolated nucleic acid sequence comprising from about 10 to about 300 consecutive nucleotides from any one of SEQ ID NO: 1 or a sequence complementary to SEQ ID NO: 1. In some embodiments, the invention is directed to an isolated nucleic acid sequence comprising from about 10 to about 400 consecutive nucleotides from SEQ ID NO: 1 or a sequence complementary to SEQ ID NO: 1. In some embodiments, the invention is directed to an isolated nucleic acid sequence comprising from about 10 to about 500 consecutive nucleotides from any one of SEQ ID NO: 1 or a sequence complementary to SEQ ID NO: 1. In some embodiments, the invention is directed to an isolated nucleic acid sequence comprising from about 10 to about 600 consecutive nucleotides from any one of SEQ ID NO: 1 or a sequence complementary to SEQ ID NO: 1. In some embodiments, the invention is directed to an isolated nucleic acid sequence comprising from about 10 to about 700 consecutive nucleotides from any one of SEQ ID NO: 1 or a sequence complementary to SEQ ID NO: 1. In some embodiments, the invention is directed to an isolated nucleic acid sequence comprising from about 10 to about 800 consecutive nucleotides from any one of SEQ ID NO: 1 or a sequence complementary to SEQ ID NO: 1. In some embodiments, the invention is directed to an isolated nucleic acid sequence comprising from about 10 to about 900 or more consecutive nucleotides from any one of SEQ ID NO: 1 or a sequence complementary to SEQ ID NO: 1.

The sequence identities can be determined by analysis with a sequence comparison algorithm. Nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

An example of an algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:402-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (www ncbi.nlm.nih.gov/). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Percent identity in the context of two or more nucleic acids, refers to the percentage of nucleotides that two or more sequences or subsequences contain which are the same. A specified percentage of nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

In some embodiments, the enhancer sequence drives expression of the polypeptide of interest or oligonucleotide of interest in a cholinergic neuron. In some embodiments, the cholinergic neuron is a motor neuron. In some embodiments, the cholinergic neuron is a basal forebrain cholinergic neuron. In some embodiments, the enhancer sequence drives expression of the polypeptide of interest or oligonucleotide of interest in a cholinergic neuron of an adult subject.

In some embodiments, the enhancer sequence is at least 80%, identical to SEQ ID NO: 1. In some embodiments, the enhancer sequence is at least 85%, identical to SEQ ID NO: 1. In some embodiments, the enhancer sequence is at least 90%, identical to SEQ ID NO: 1. In some embodiments, the enhancer sequence is at least 95%, identical to SEQ ID NO: 1. In some embodiments, the enhancer sequence is at least 96%, identical to SEQ ID NO: 1. In some embodiments, the enhancer sequence is at least 97%, identical to SEQ ID NO: 1. In some embodiments, the enhancer sequence is at least 98%, identical to SEQ ID NO: 1. In some embodiments, the enhancer sequence is at least 99%, identical to SEQ ID NO: 1. In some embodiments, the enhancer sequence comprises SEQ ID NO: 1. In some embodiments, the enhancer sequence consists of SEQ ID NO: 1.

In some embodiments, the nucleic acid comprises a nucleotide sequence encoding a polypeptide of interest, wherein the polypeptide of interest is a prophylactic or therapeutic polypeptide, as described further herein. See e.g., section titled Polypeptides of Interest and RNAs of Interest below.

In some embodiments, the nucleic acid further comprises a nucleotide sequence encoding a selectable marker gene. In some embodiments, the selectable marker gene is an antibiotic resistance gene. In some embodiments, the antibiotic resistance gene is an ampicillin resistance gene. In some embodiments, the antibiotic resistance gene comprises SEQ ID NO: 2. In some embodiments, the antibiotic resistance gene comprises a nucleotide sequence encoding an amino acid sequence of SEQ ID NO: 3.

In some embodiments, the nucleic acid further comprises a nucleotide sequence encoding a promoter sequence positioned between the enhancer sequence and the nucleotide sequence encoding the polypeptide of interest or an oligonucleotide of interest. In some embodiments, the promoter sequence is a mini-promoter sequence. In some embodiments, the promoter sequence comprises SEQ ID NO: 6.

In some embodiments, the nucleic acid further comprises a nucleotide sequence of an intron sequence positioned between the enhancer sequence and the nucleotide sequence encoding the polypeptide of interest or an oligonucleotide of interest. In some embodiments, the intron is a chimeric intron. In some embodiments, the intron sequence comprises SEQ ID NO: 7.

In some embodiments, the nucleic acid further comprises a nucleotide sequence of a post-transcriptional regulatory element positioned 3′ to the nucleotide sequence encoding the polypeptide of interest. In some embodiments, the post-transcriptional regulatory element is further positioned proximal to a polyA sequence. In some embodiments, the post-transcriptional regulatory element comprises a woodchuck hepatitis virus post-transcriptional regulatory element (WPRE). In some embodiments, the post-transcriptional regulatory element comprises SEQ ID NO: 8.

In some embodiments, the nucleic acid further comprises a nucleotide sequence encoding a polypeptide of interest and further comprising a nucleotide sequence of a polyA sequence positioned 3′ to the nucleotide sequence encoding the polypeptide of interest. In some embodiments, the polyA sequence is an SV40 poly A sequence. In some embodiments, the polyA sequence comprises SEQ ID NO: 9.

### Vectors and Constructs

In other aspects, the invention is directed to expression constructs, for example but not limited to plasmids and vectors which comprise the nucleic acid sequence of SEQ ID NO: 1, complementary sequences thereof, and/or variants thereof. In certain embodiments, the expression constructs further comprise the sequence encoding the oligonucleotide or polypeptide of interest. Such expression constructs can be prepared by any suitable method known in the art. Such expression constructs are suitable for viral nucleic acid and/or protein expression and purification.

In certain aspects, the invention provides a nucleic acid vector comprising: an enhancer sequence comprising a nucleotide sequence at least 70% identical to SEQ ID NO: 1; and a nucleotide sequence encoding a polypeptide of interest or an oligonucleotide of interest, wherein the nucleotide encoding the polypeptide of interest or oligonucleotide of interest is positioned 3′ to the enhancer sequence. In certain embodiments, the nucleic acid vector comprises the nucleic acid features described above. See section titled “Nucleic Acids”.

In certain embodiments, the vector is a viral vector. In some embodiments, the vector is circular, and in other embodiments, the vector is linearized. Viral vectors include adeno-associated viral (AAV), adenoviral, lentiviral, and retroviral vectors. AAVs can infect terminally differentiated cells, establish nuclear episomes without risking insertional mutagenesis, and convey long-term transgene expression and mild immune responses, making them a preferred choice of delivery to neurons. In certain embodiments, an AAV vector may convey transgene expression for at least one week, at least one month, at least four months, at least six months, at least one year, or longer.

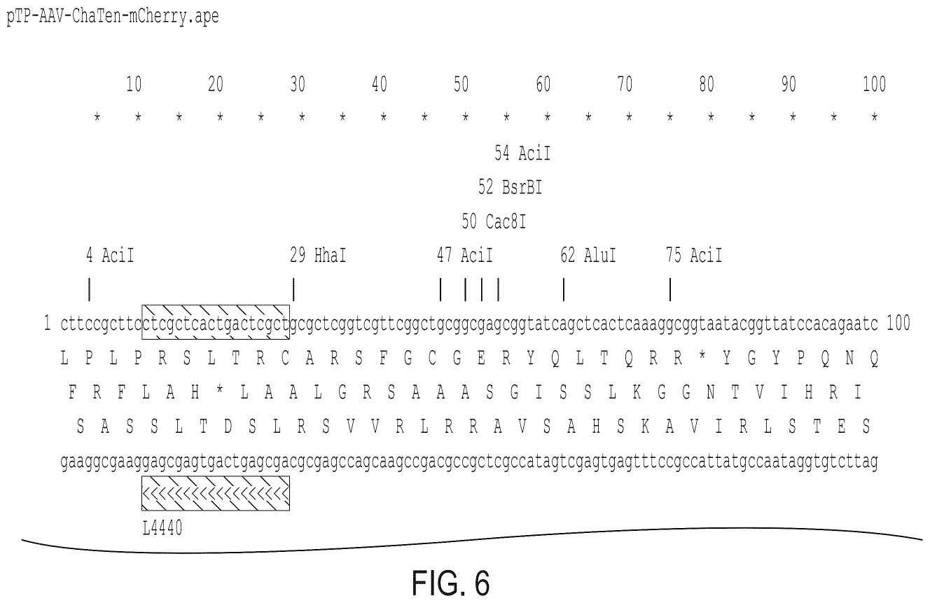

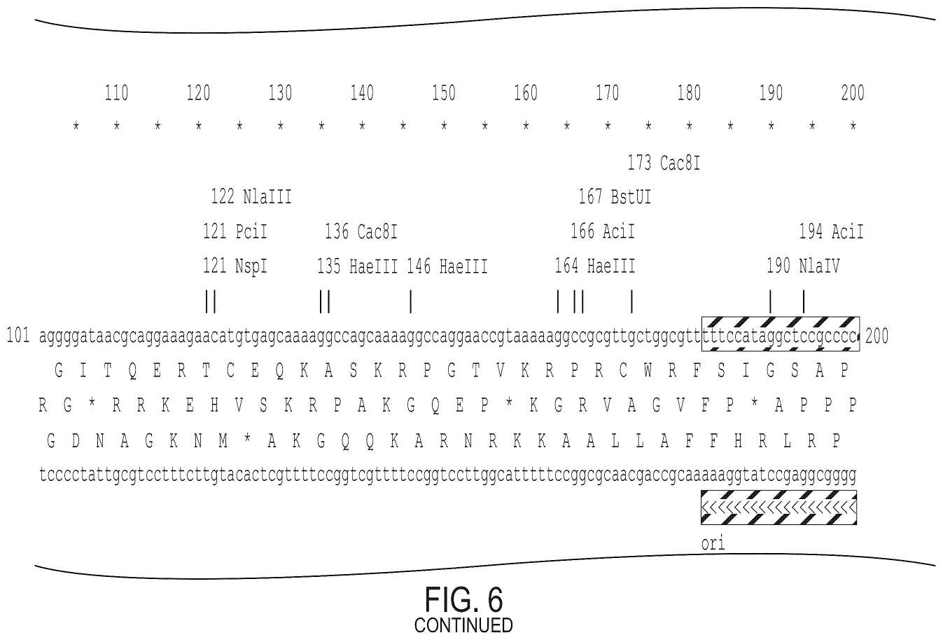

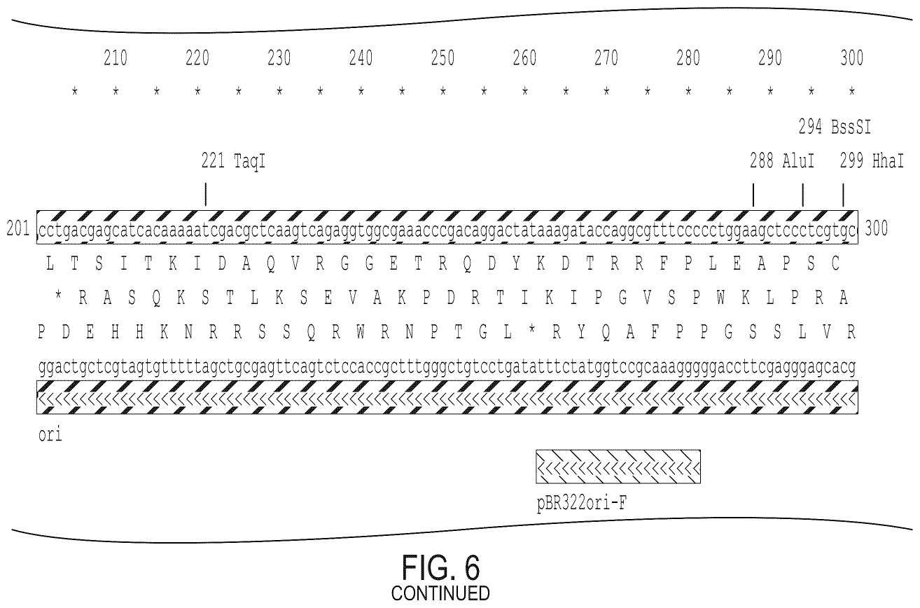

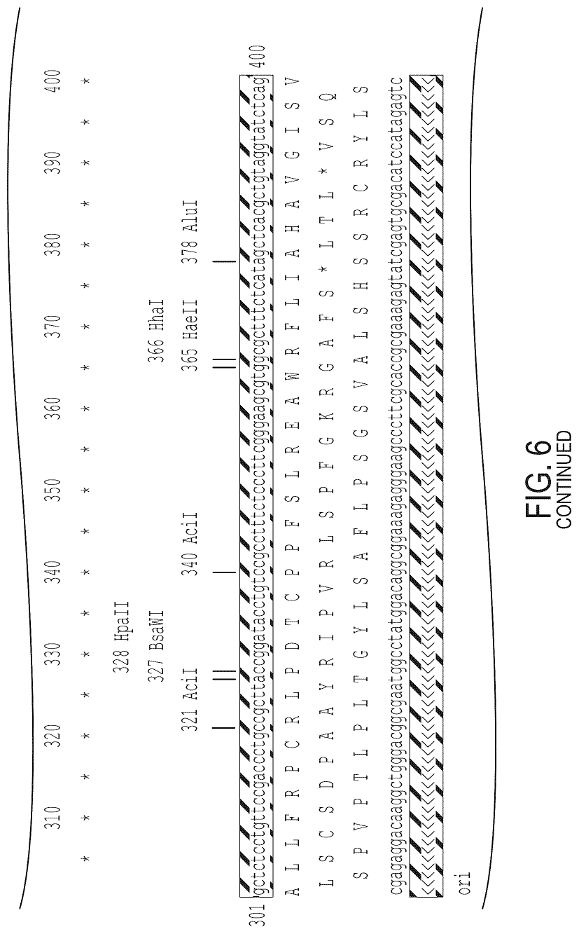

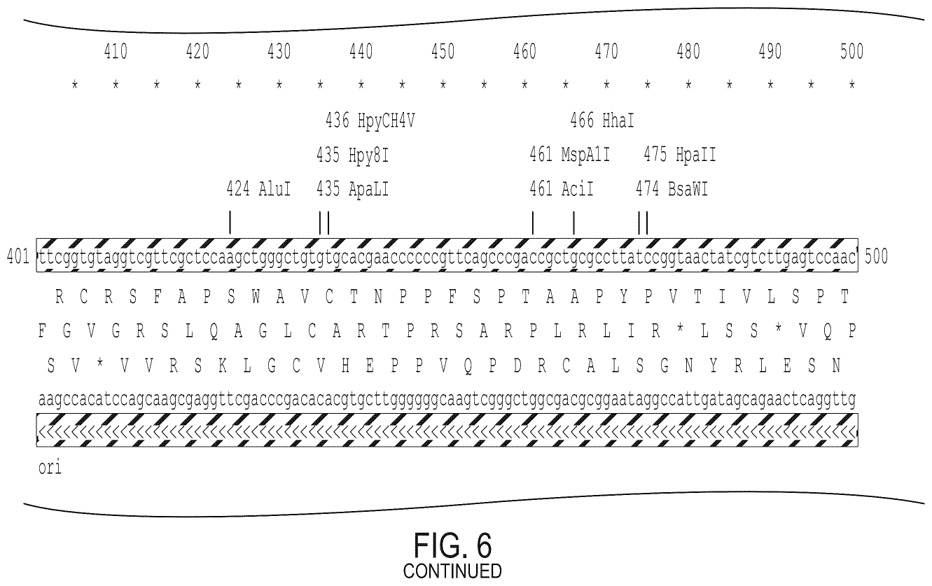

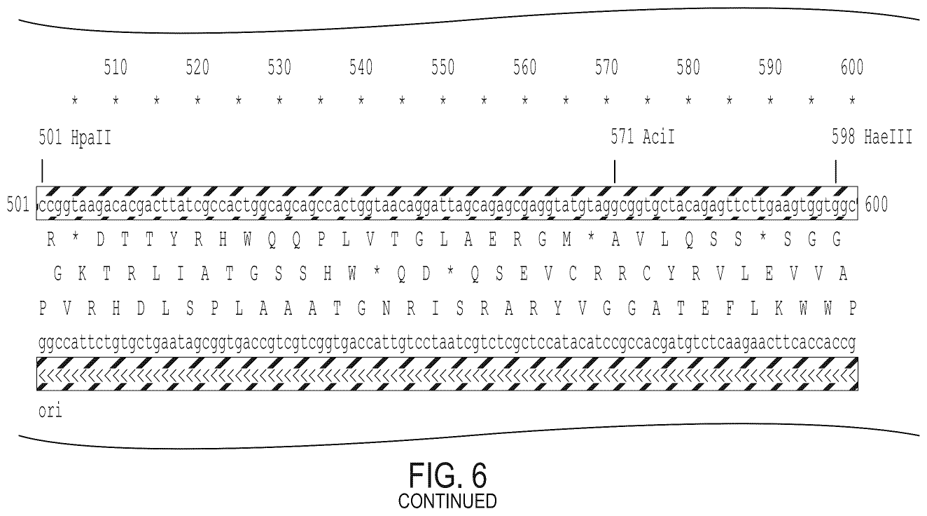

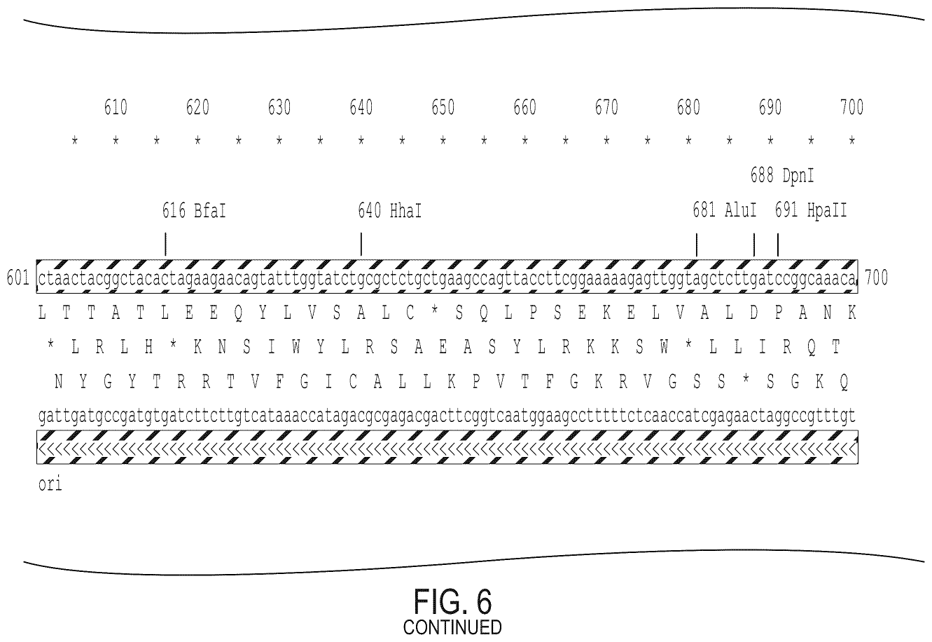

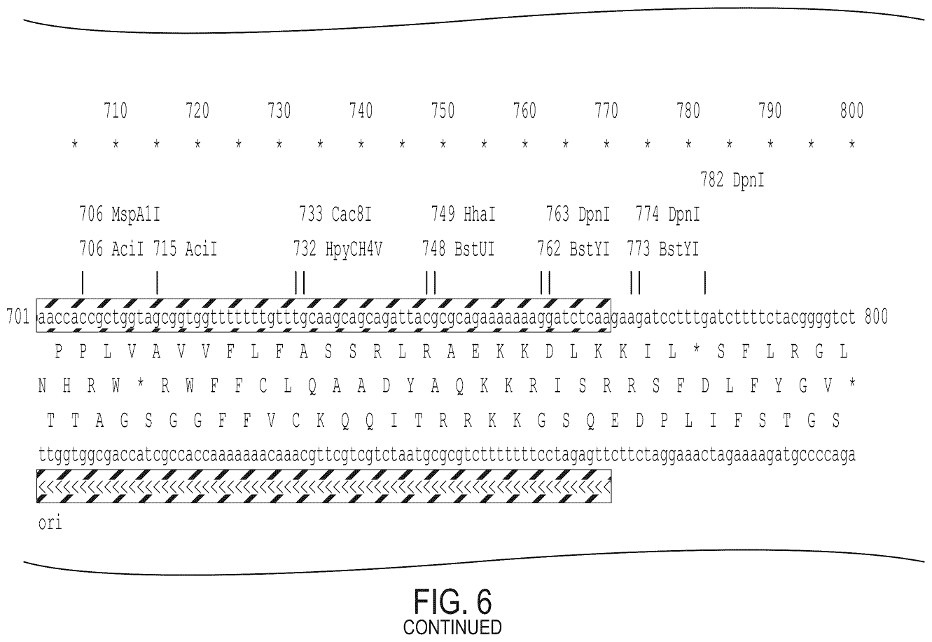

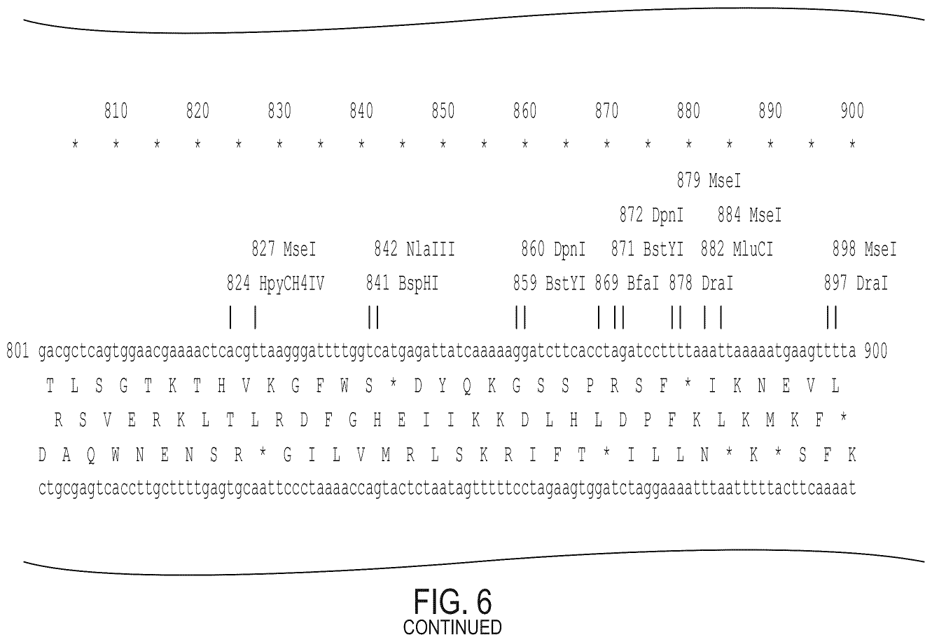

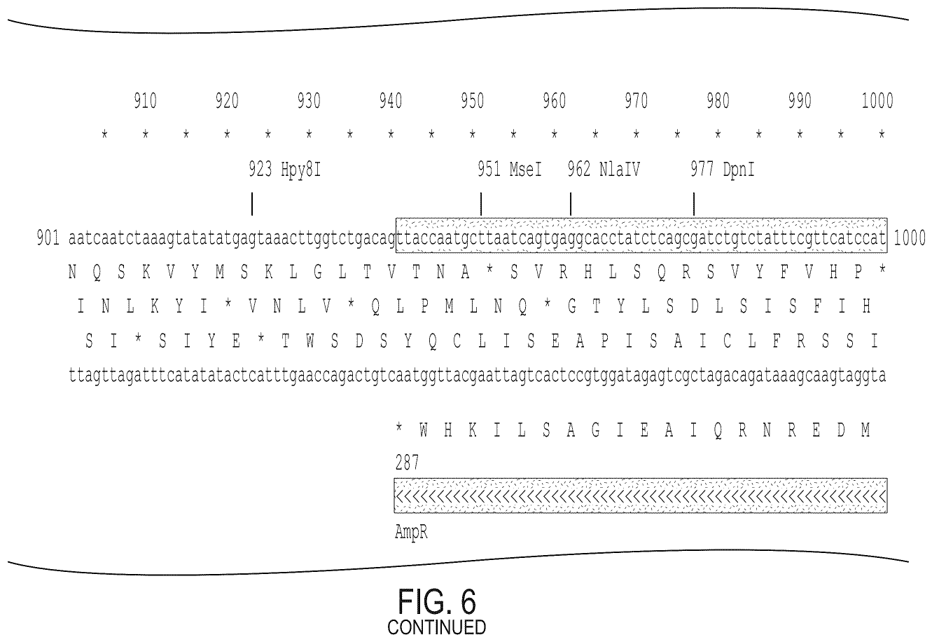

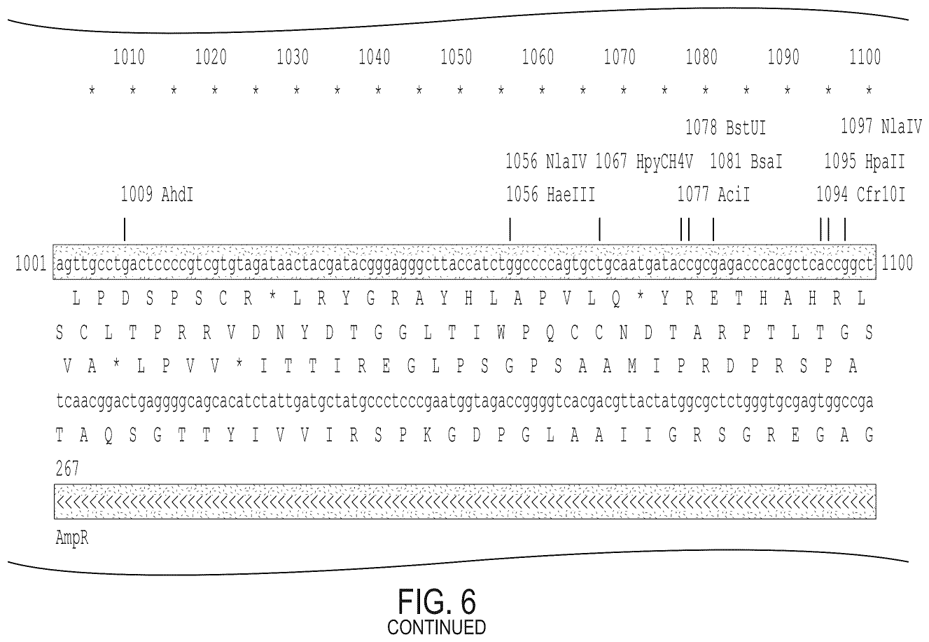

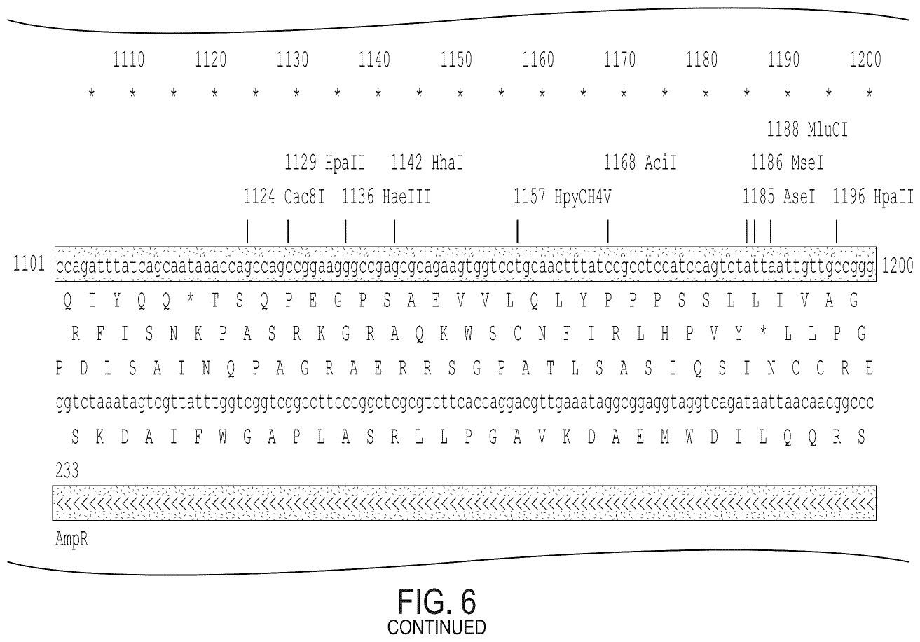

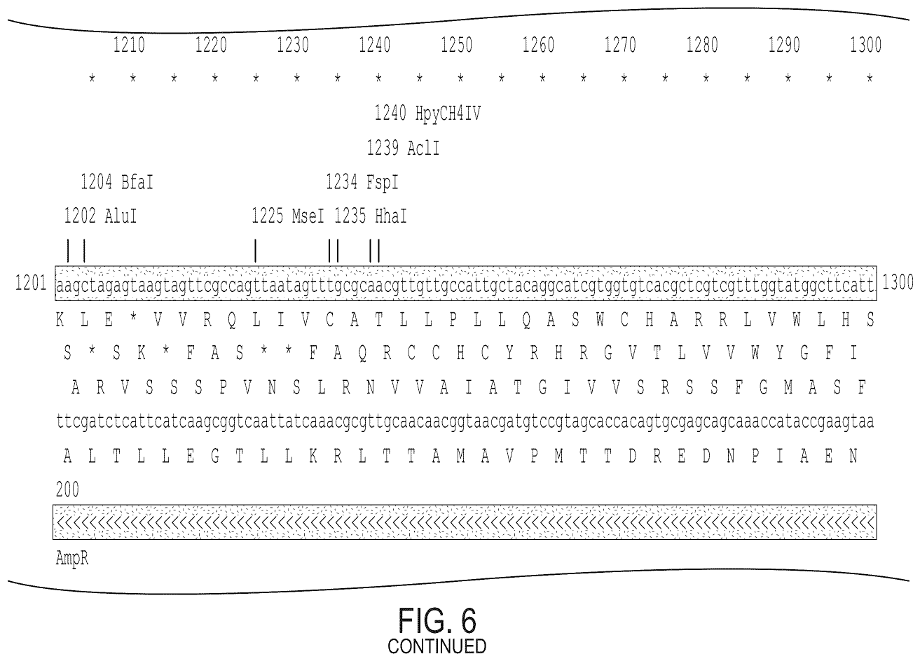

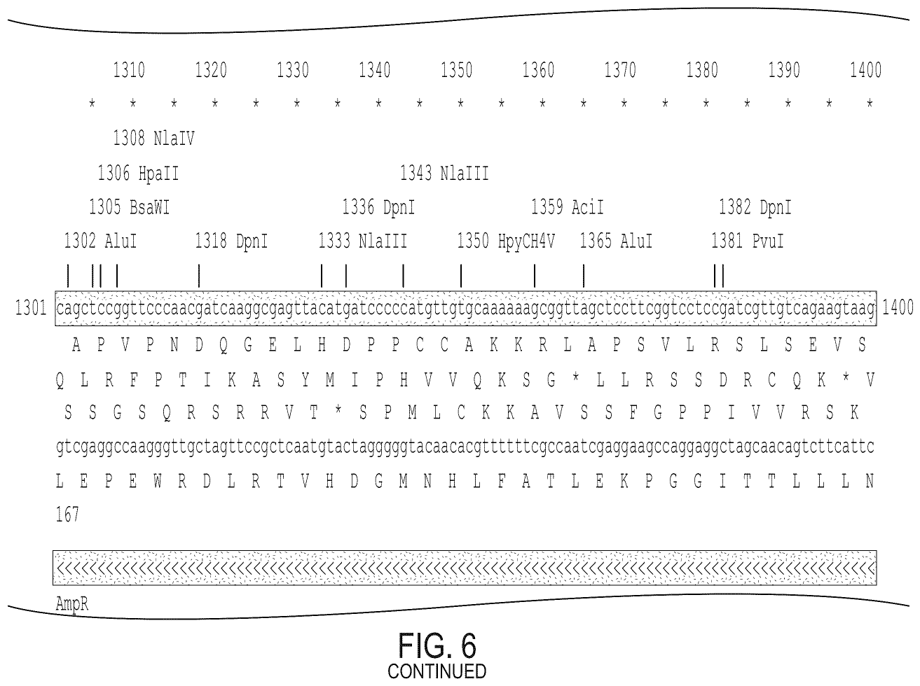

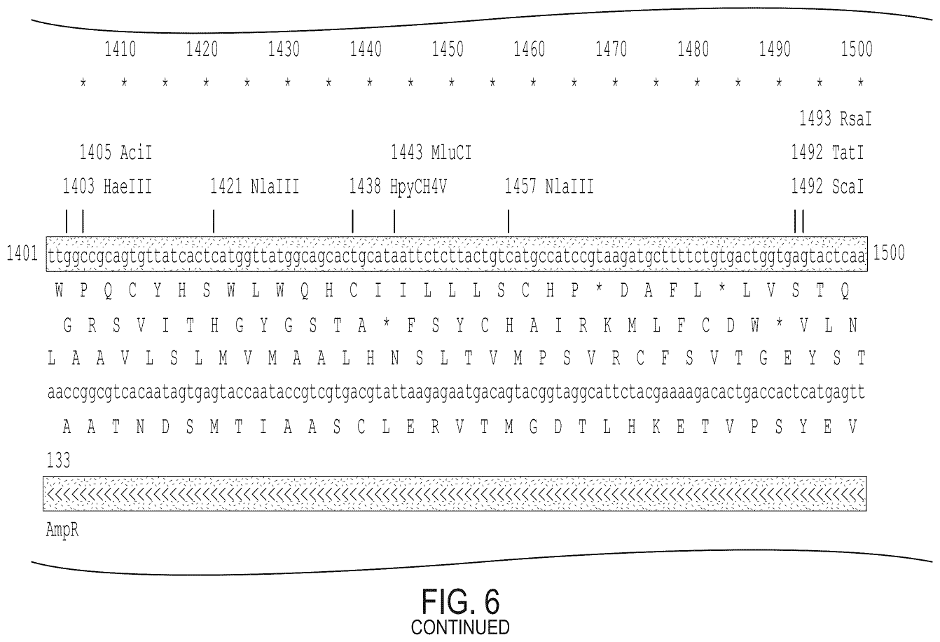

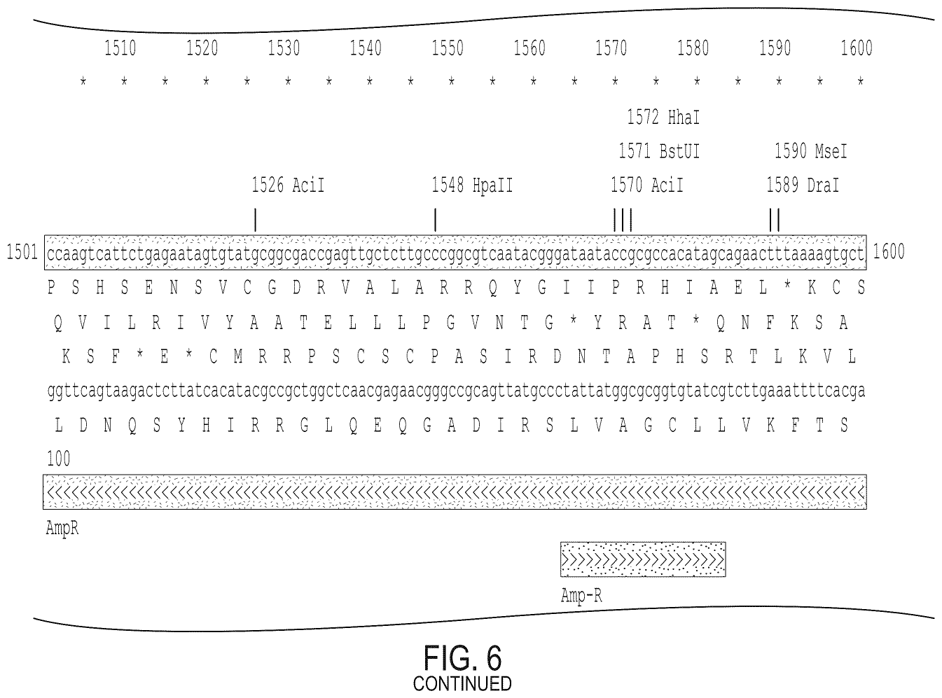

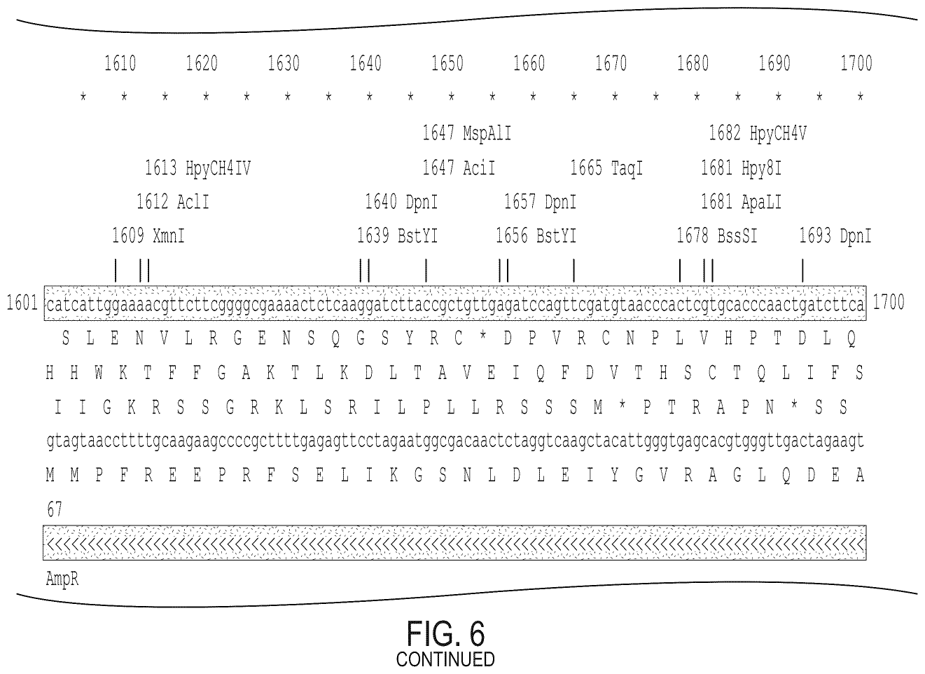

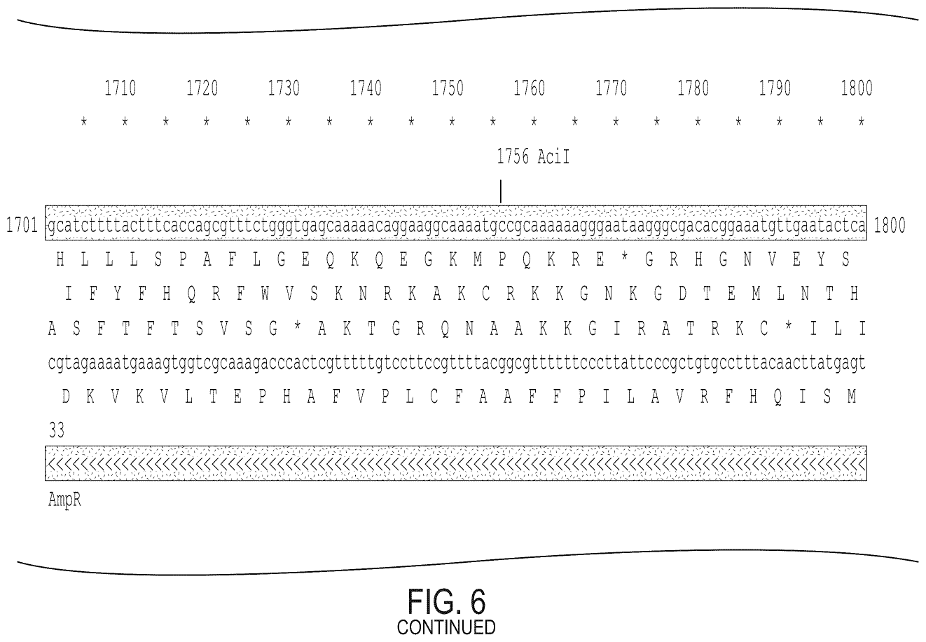

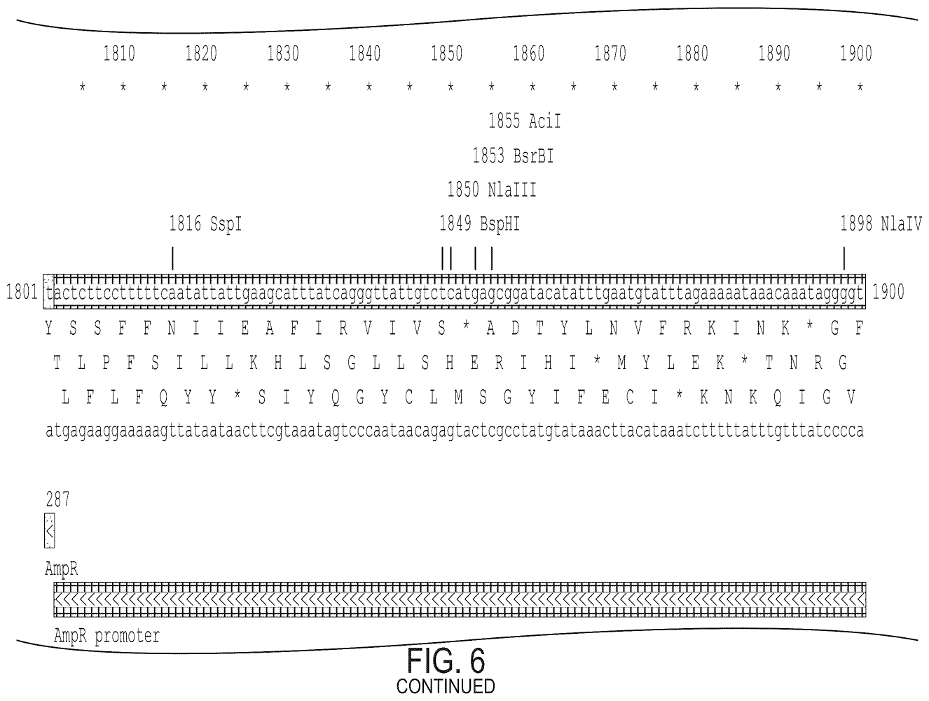

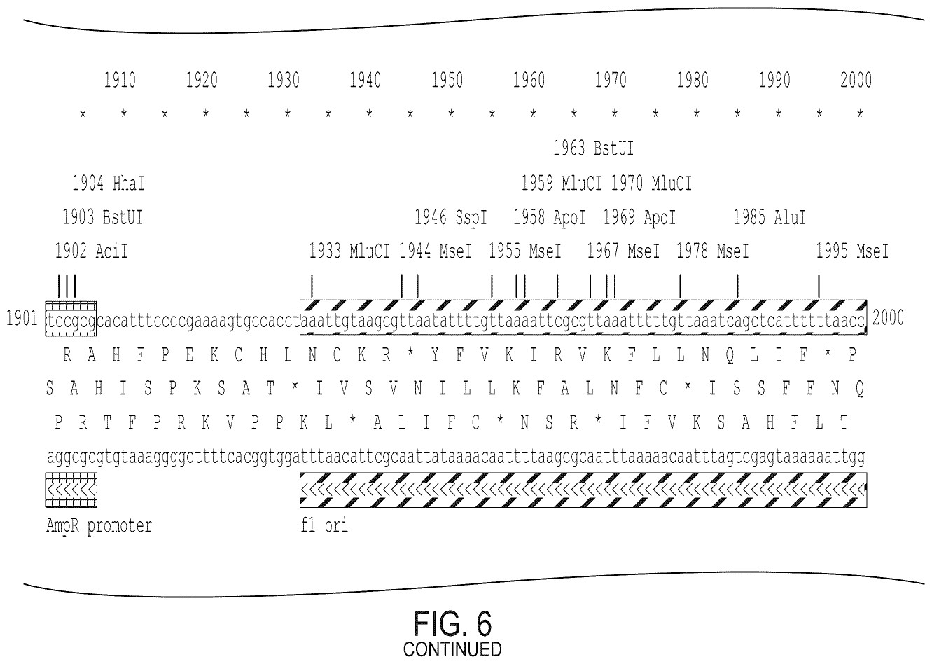

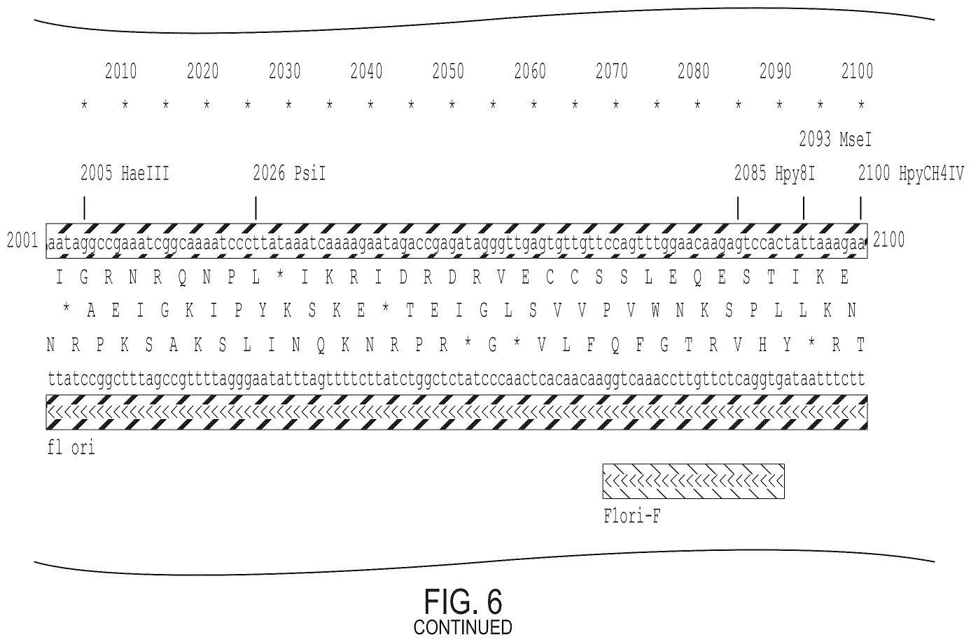

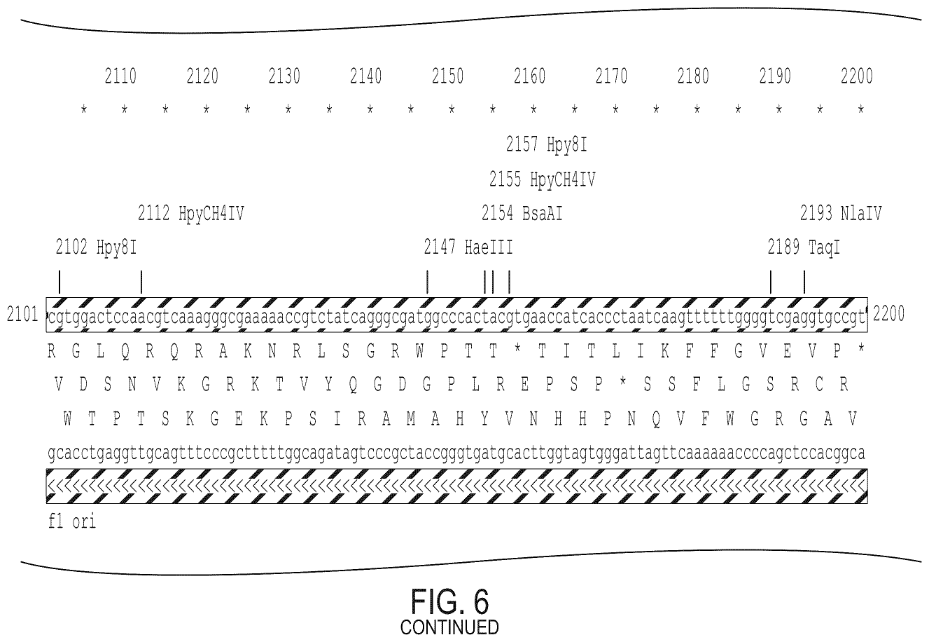

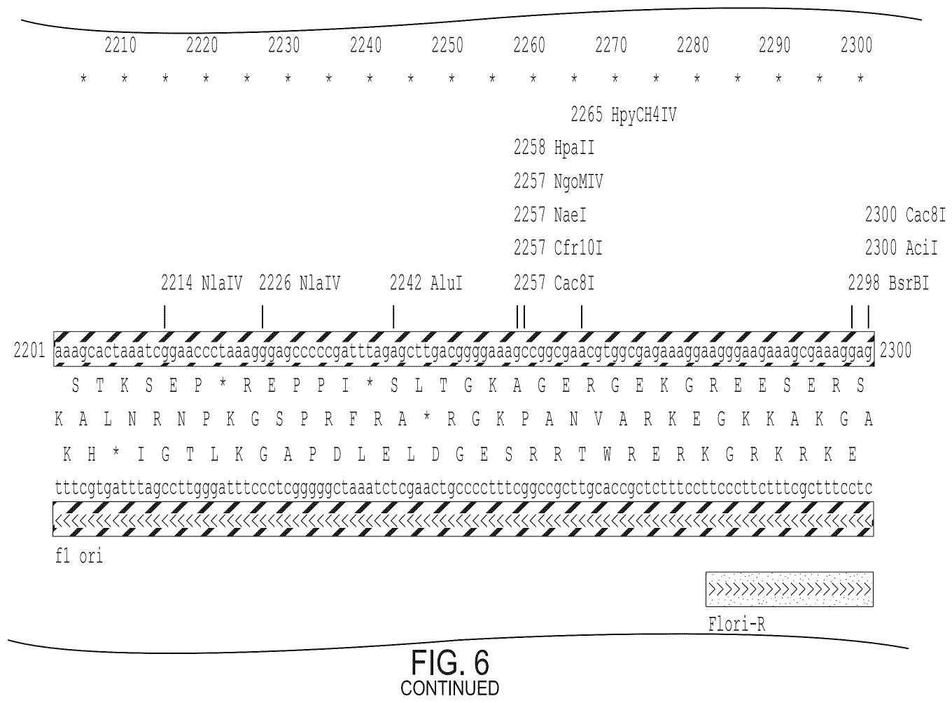

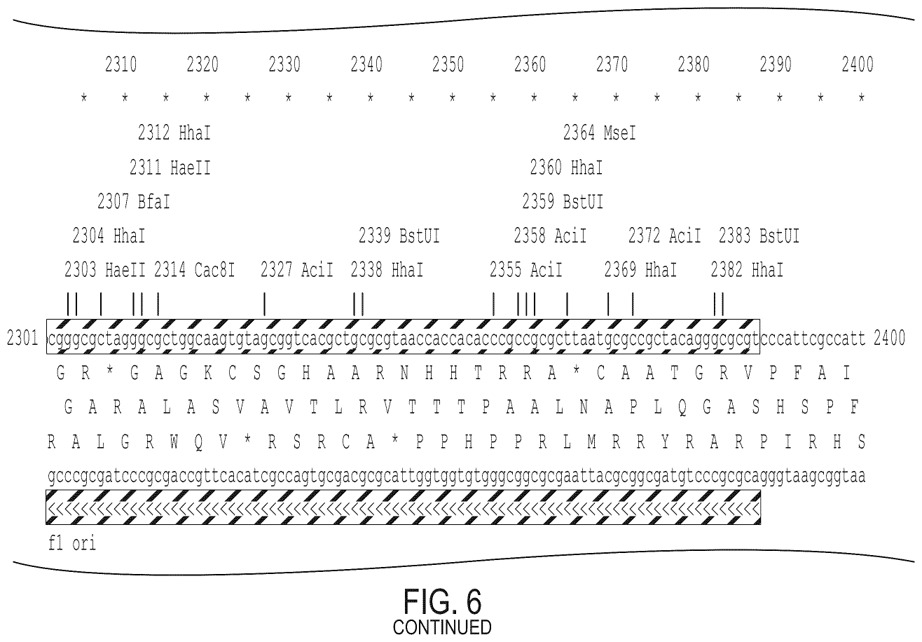

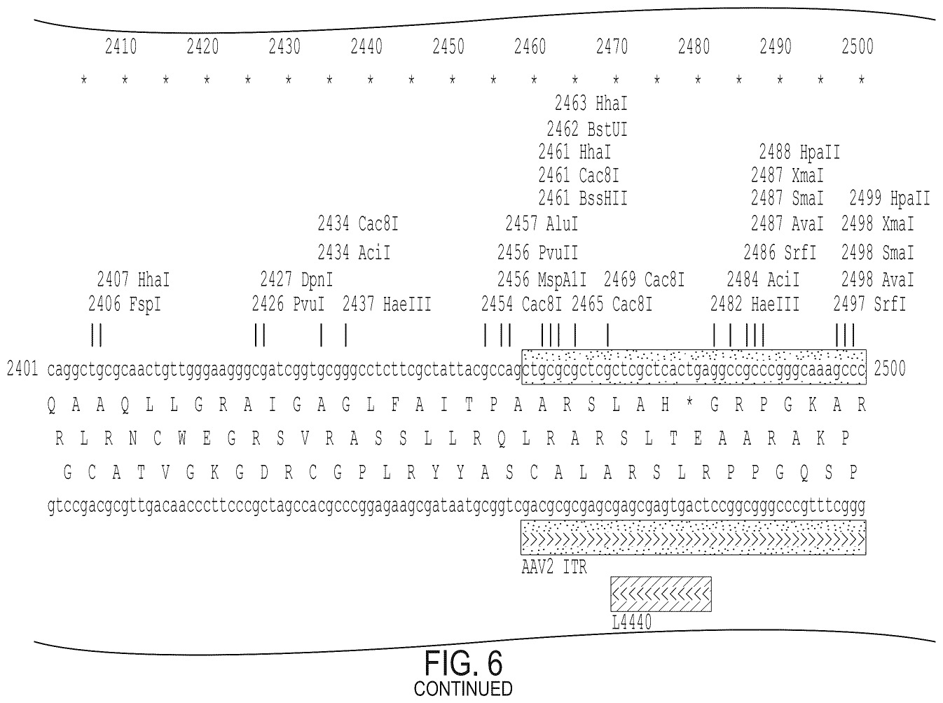

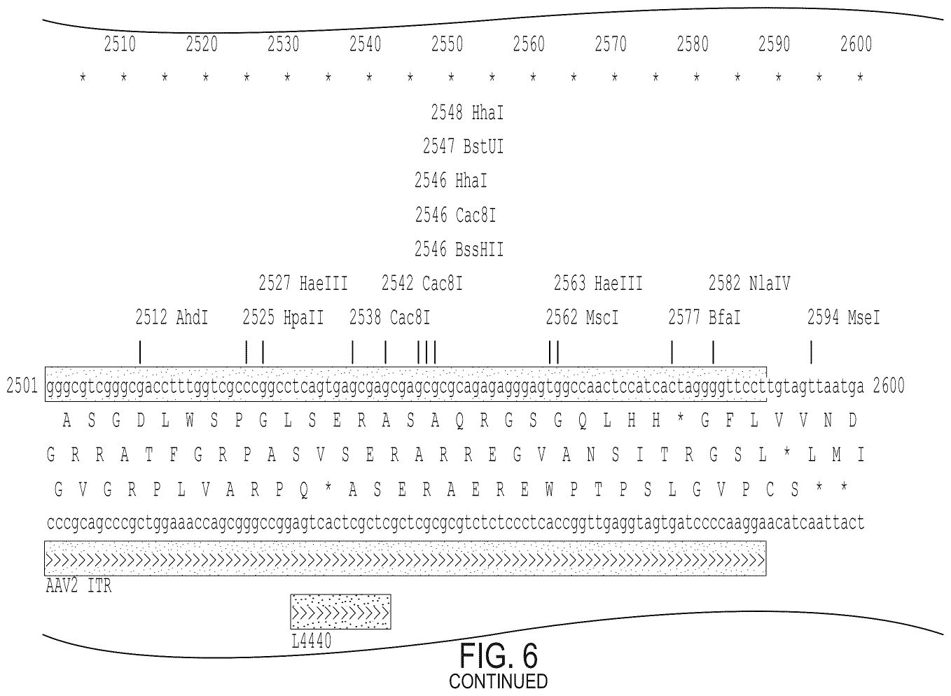

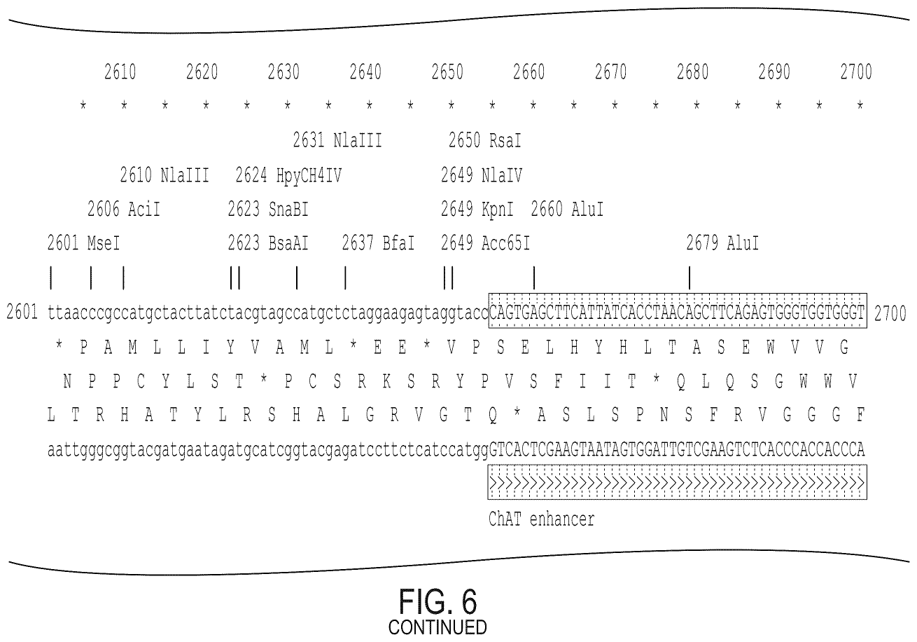

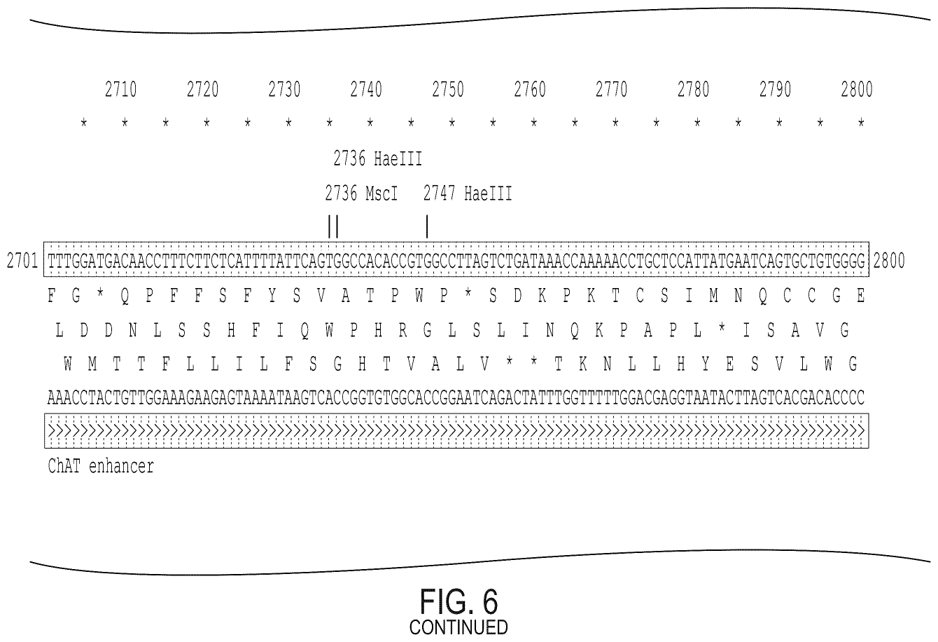

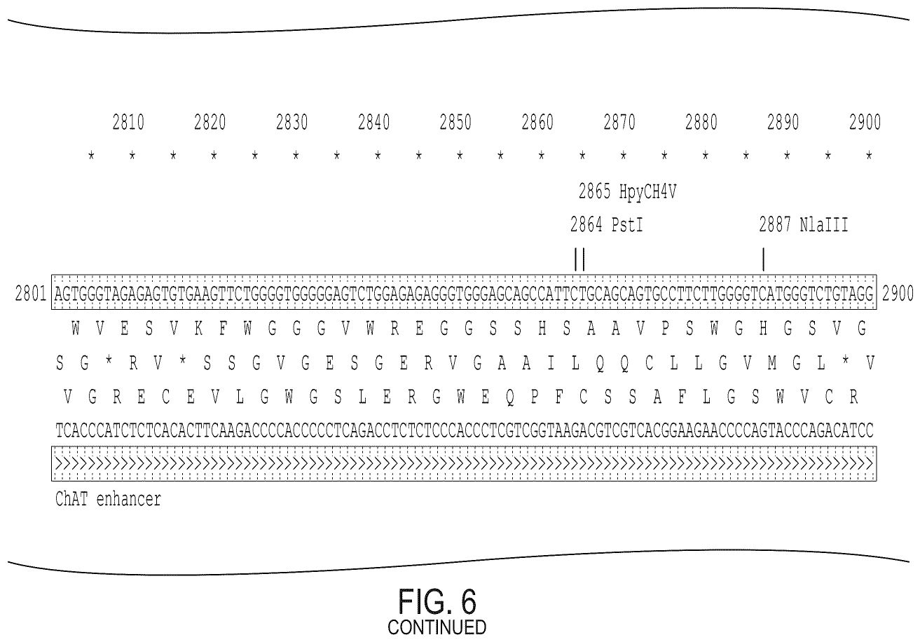

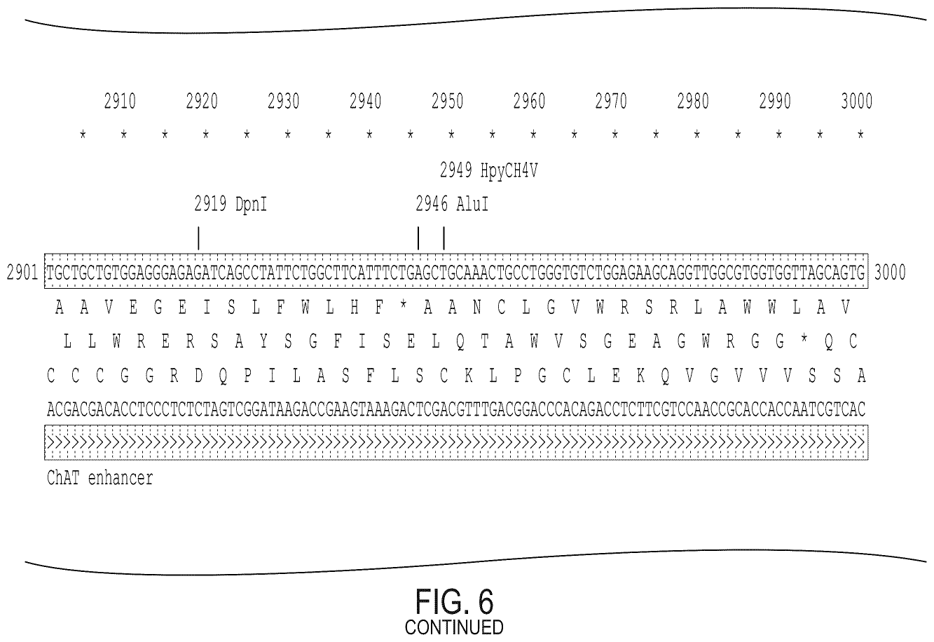

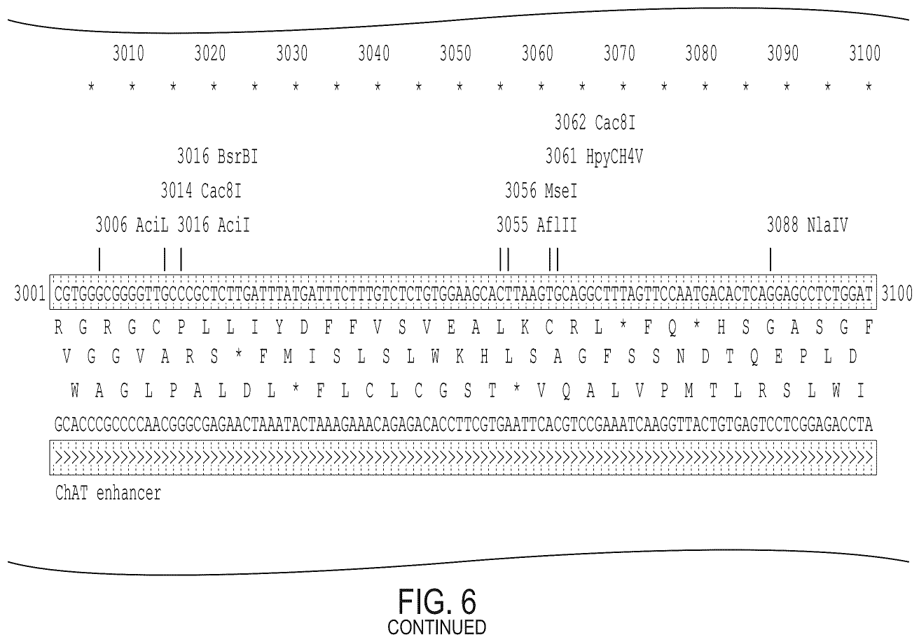

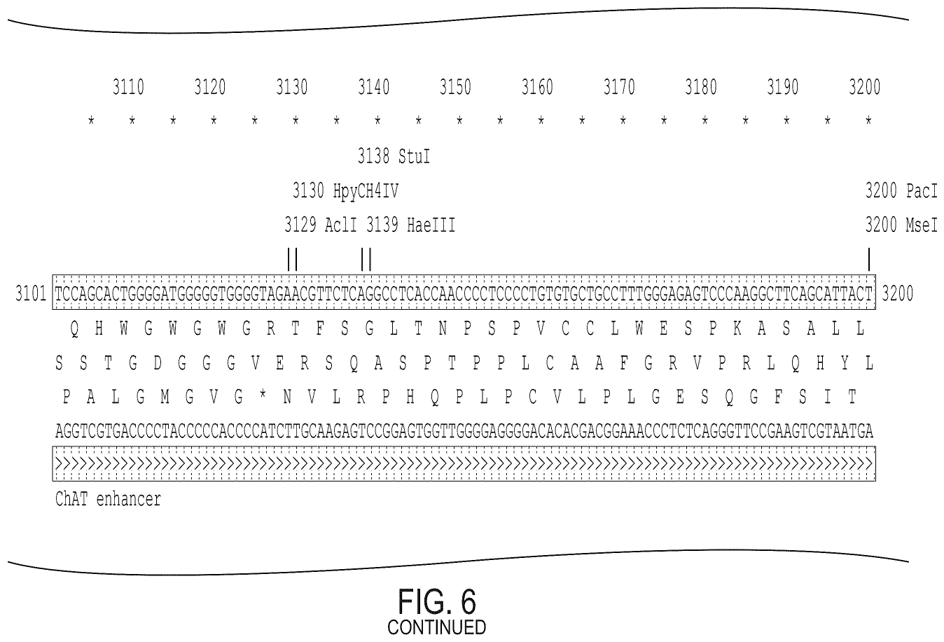

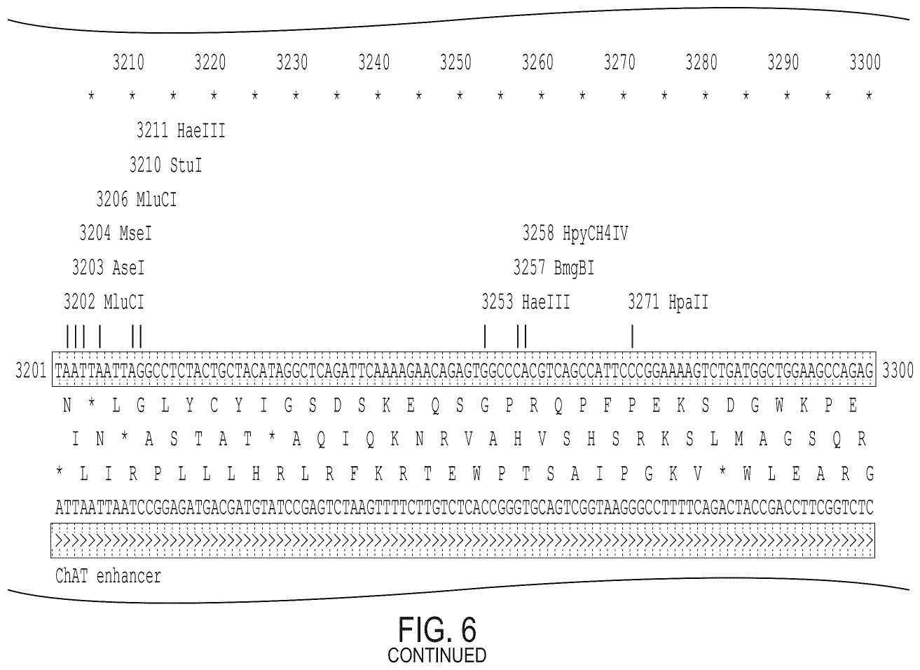

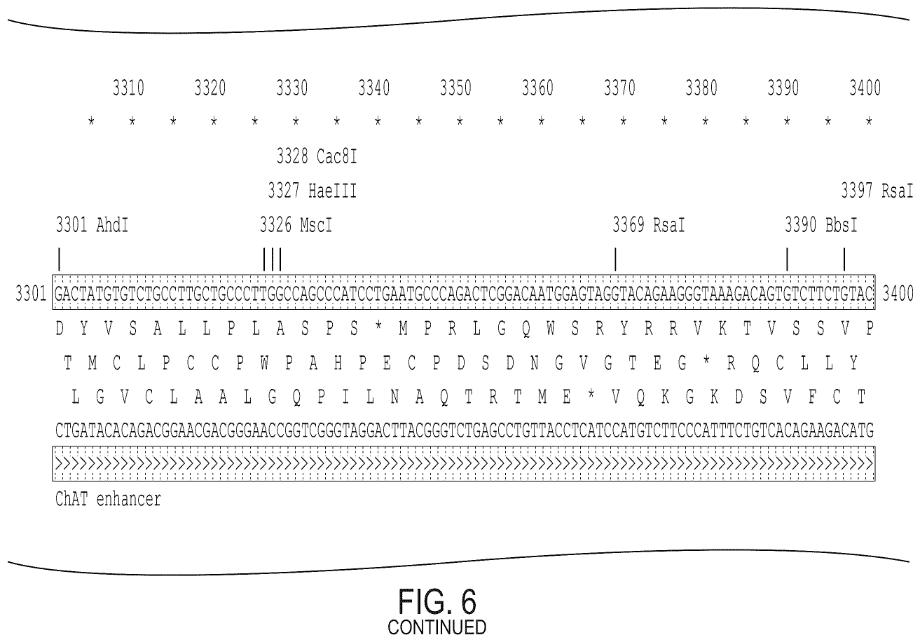

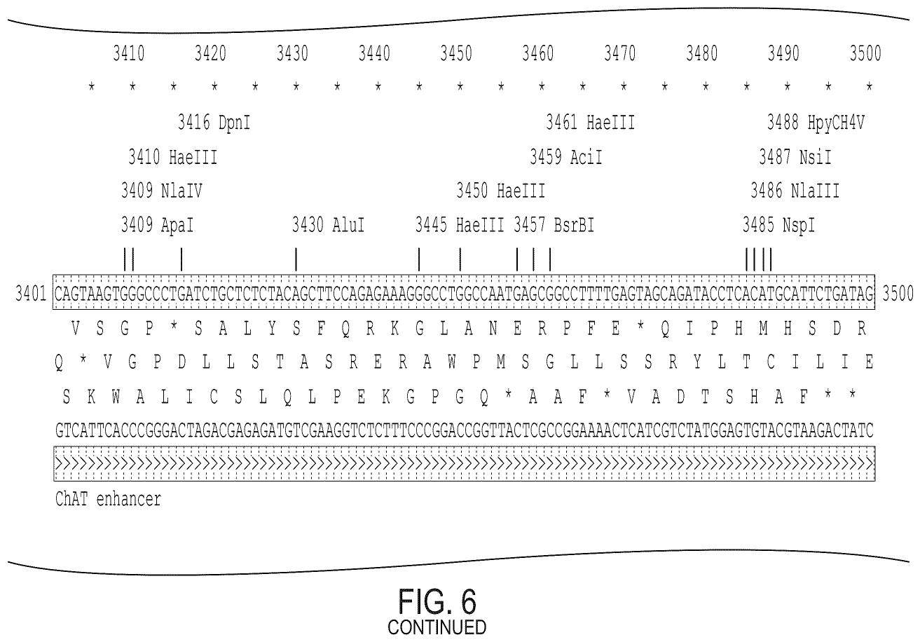

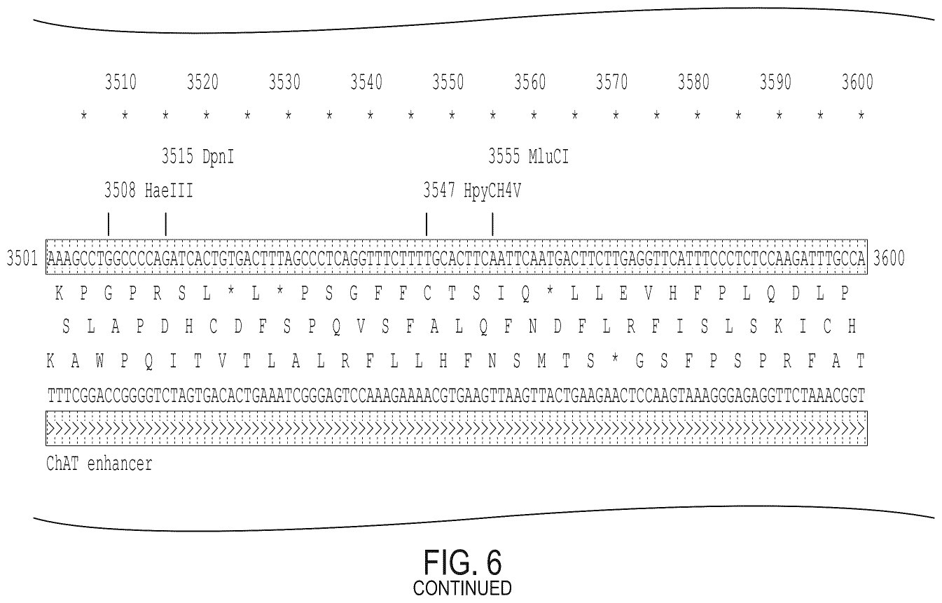

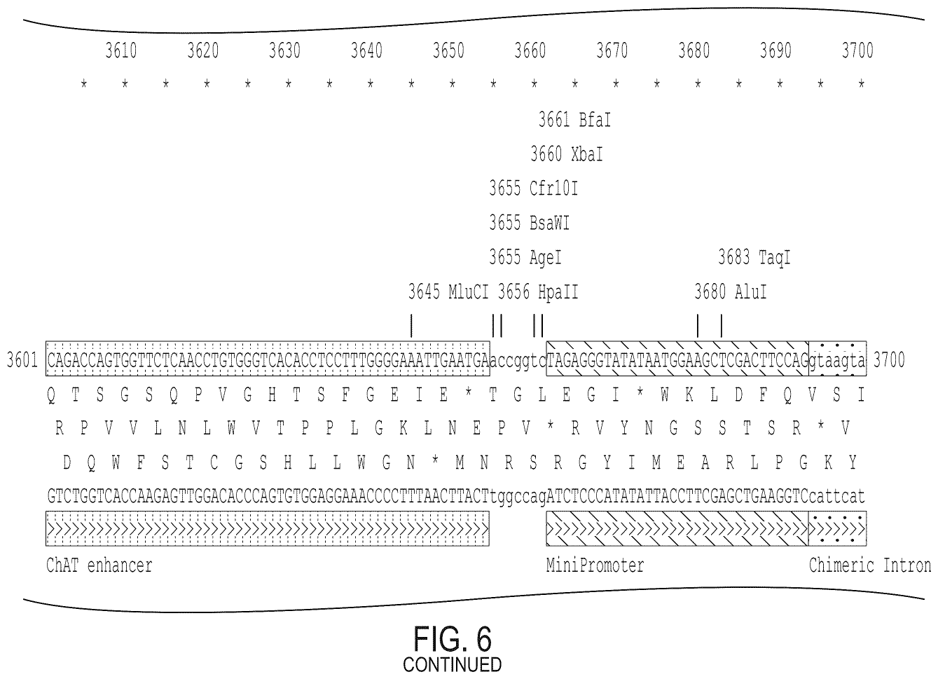

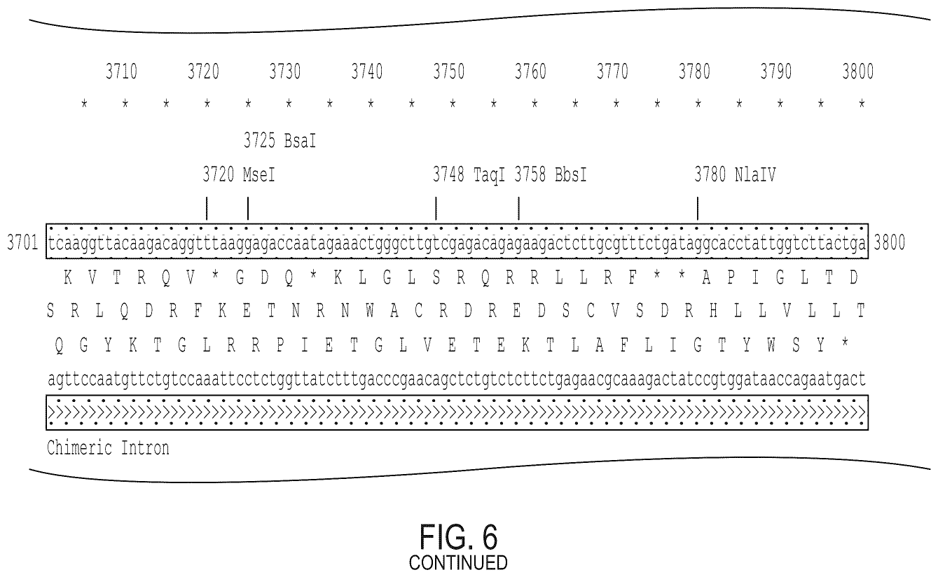

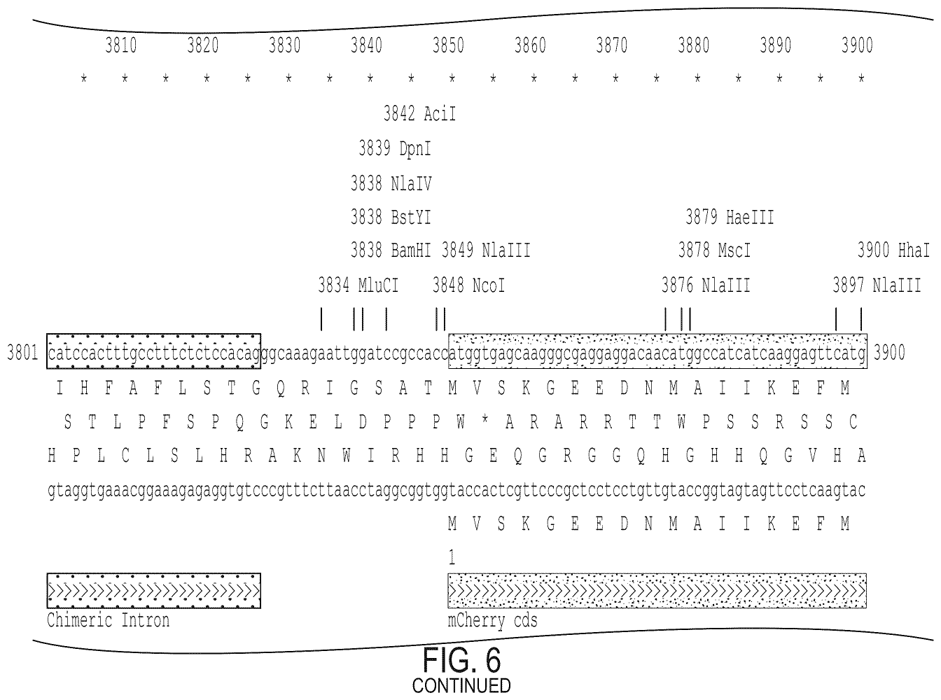

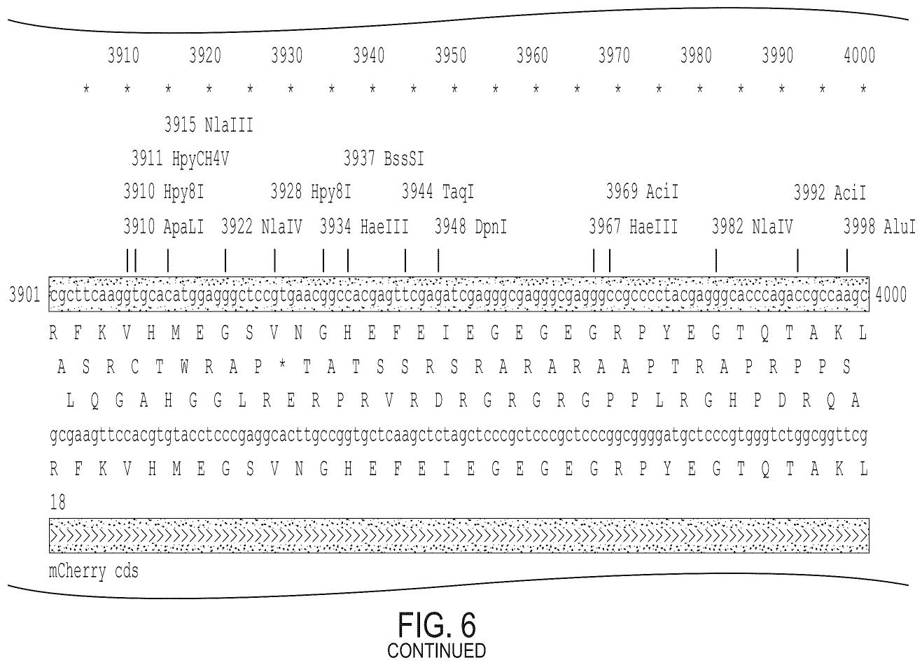

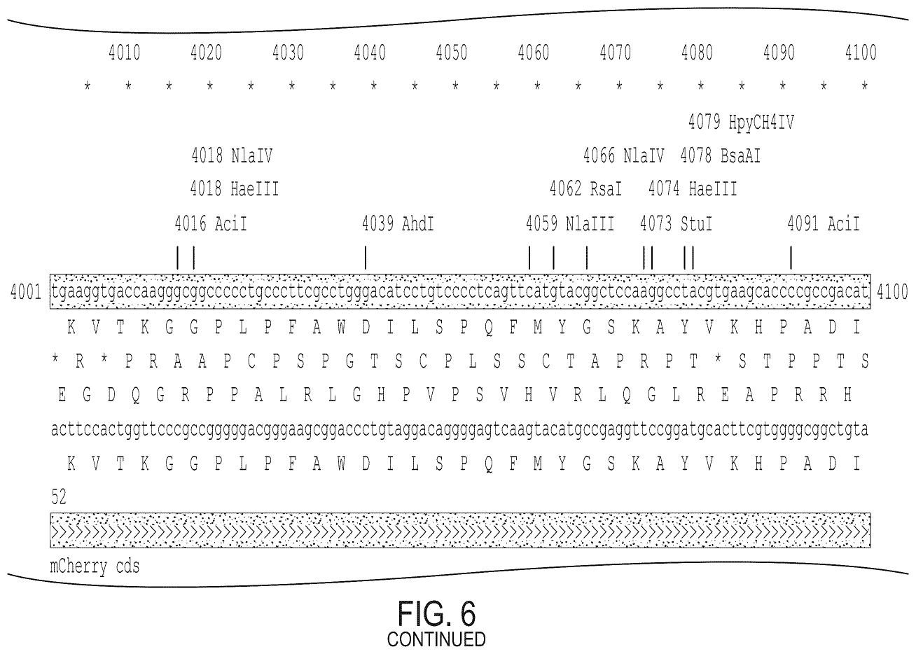

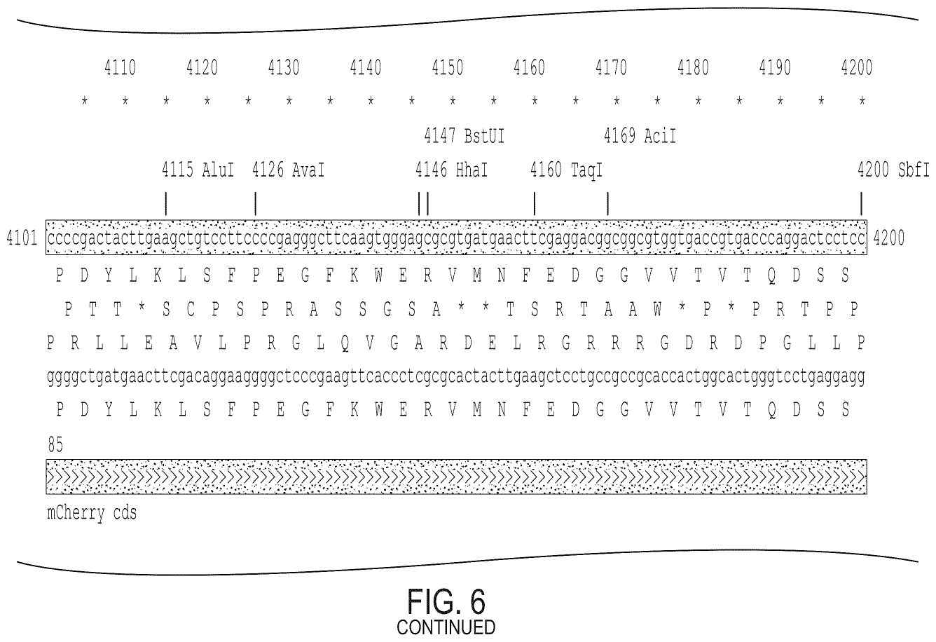

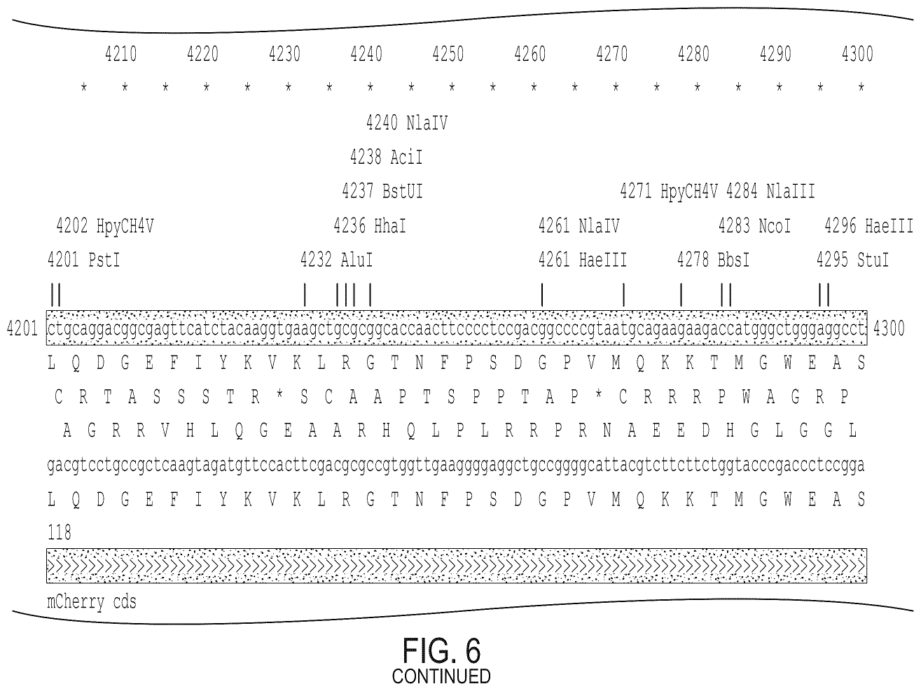

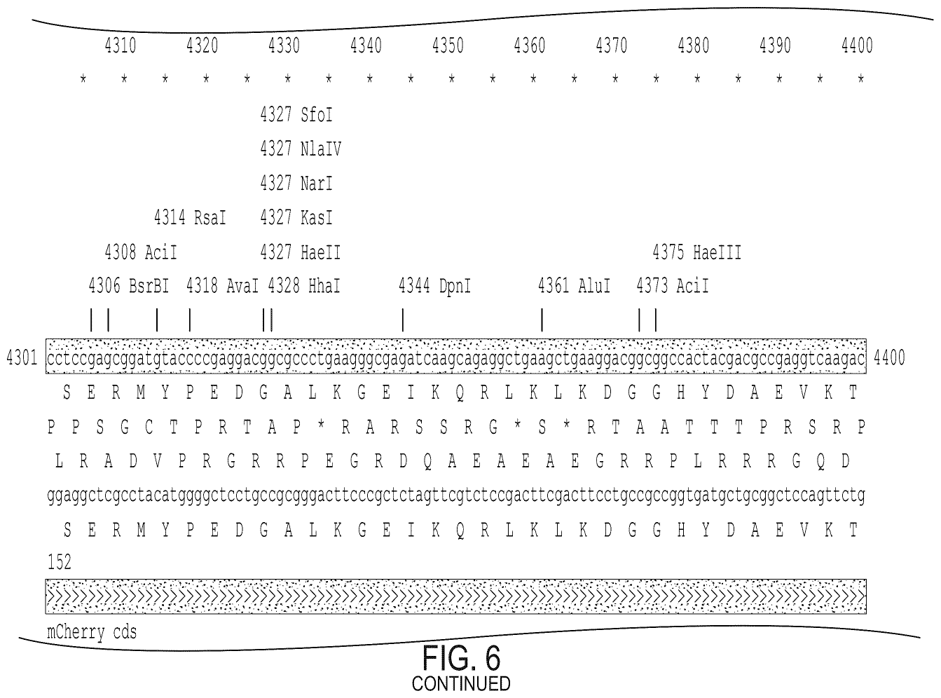

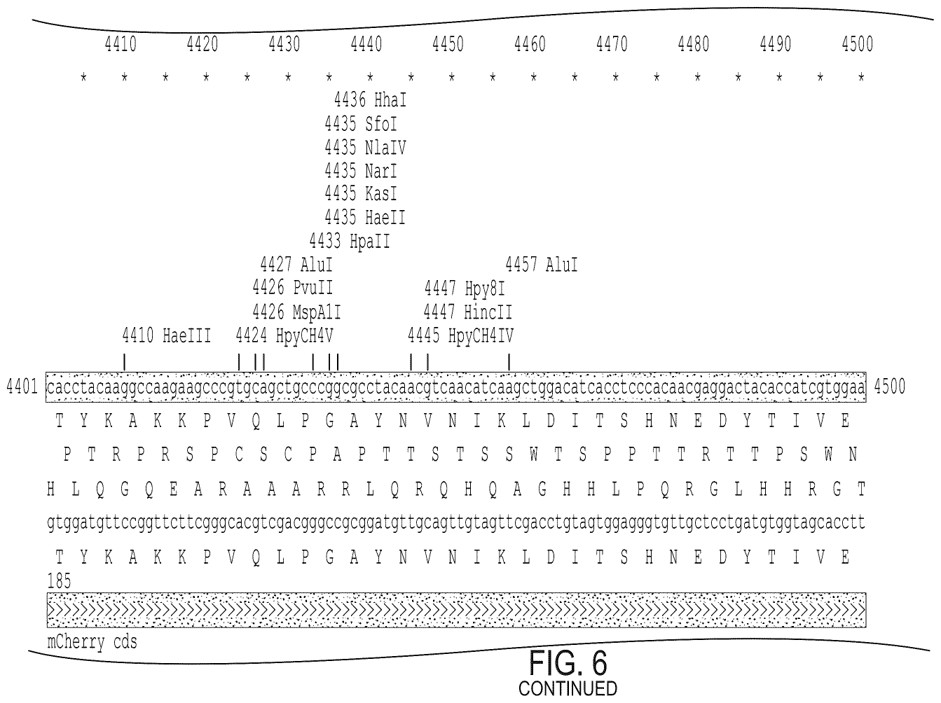

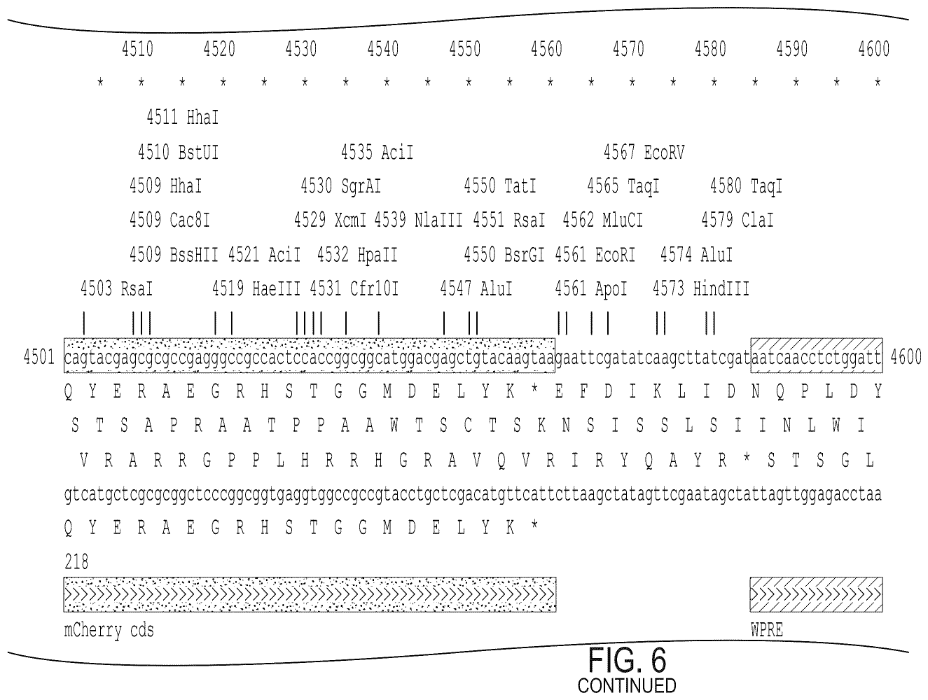

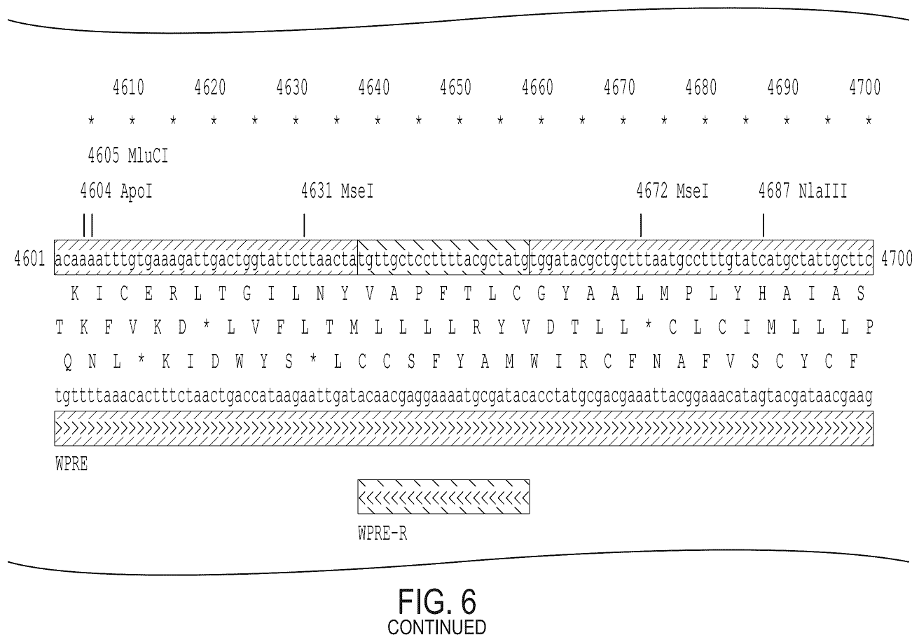

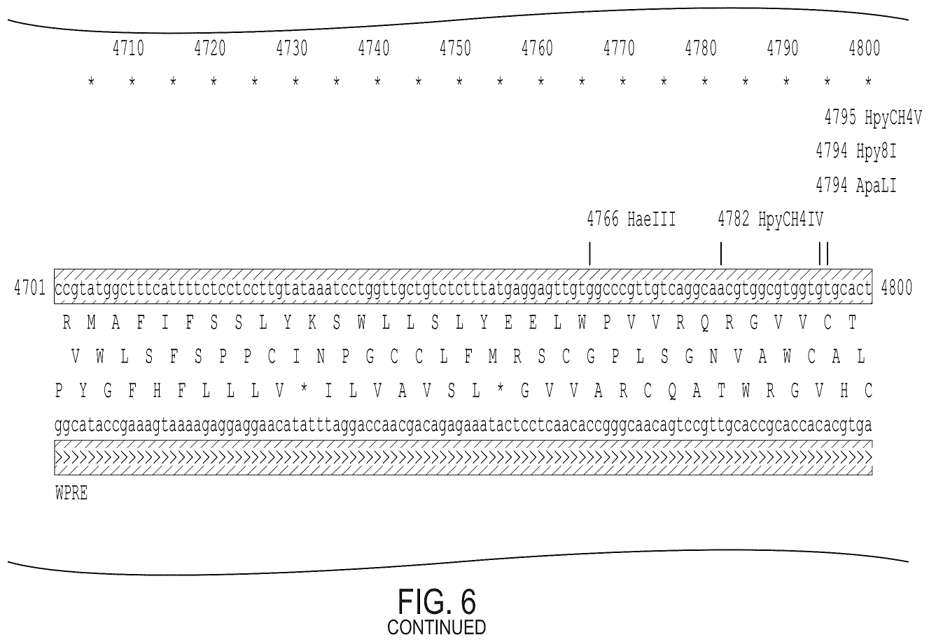

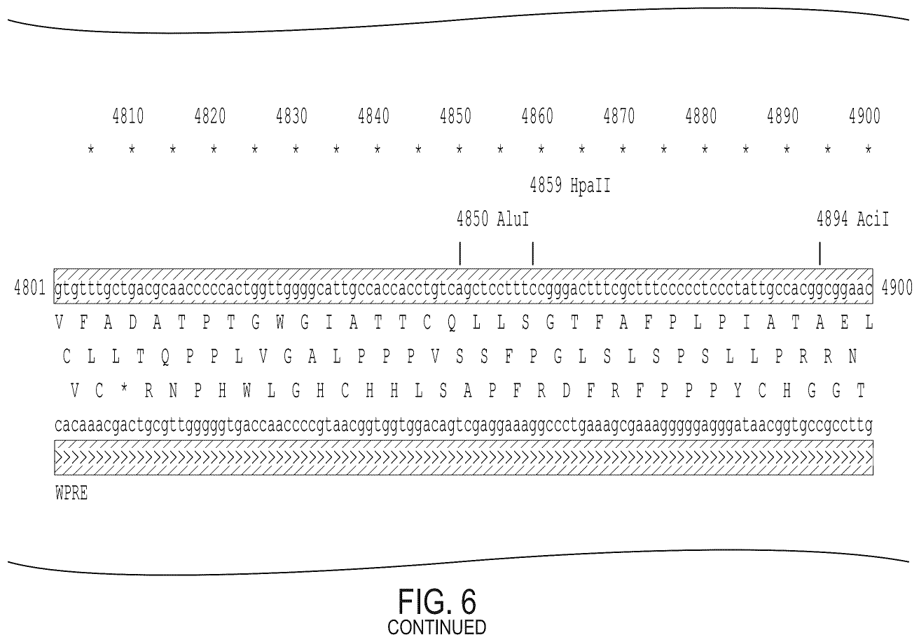

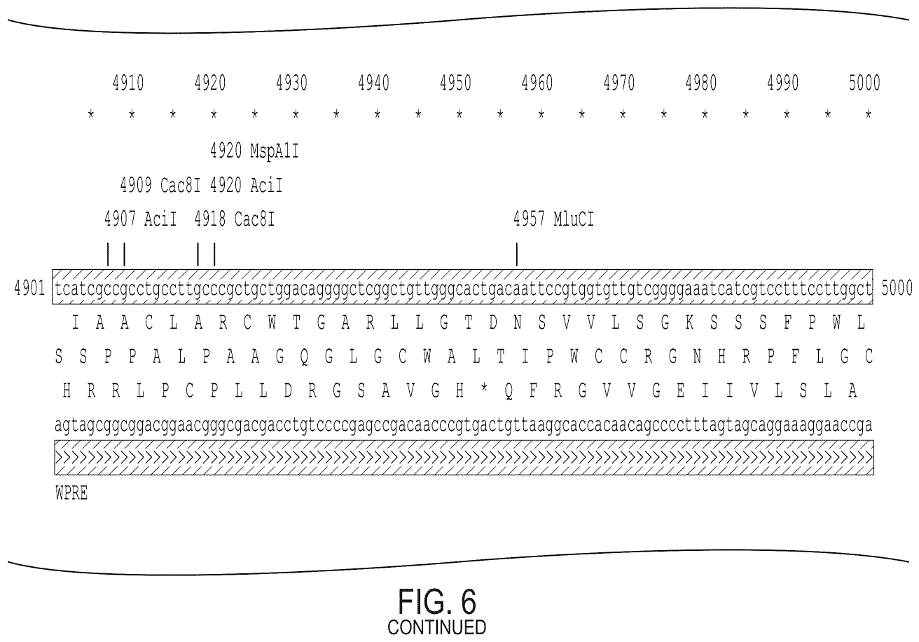

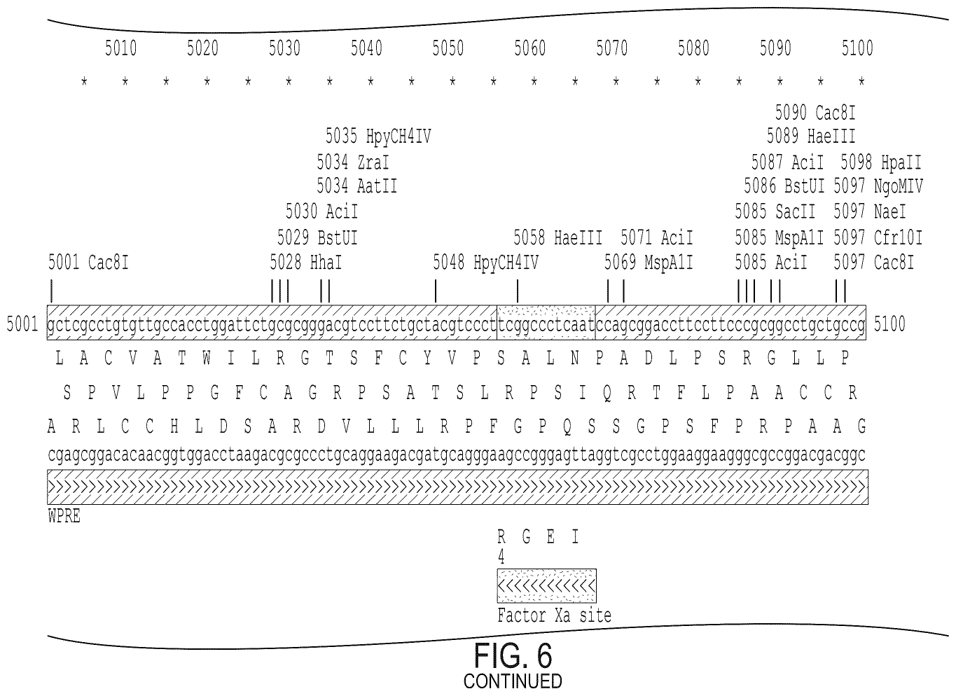

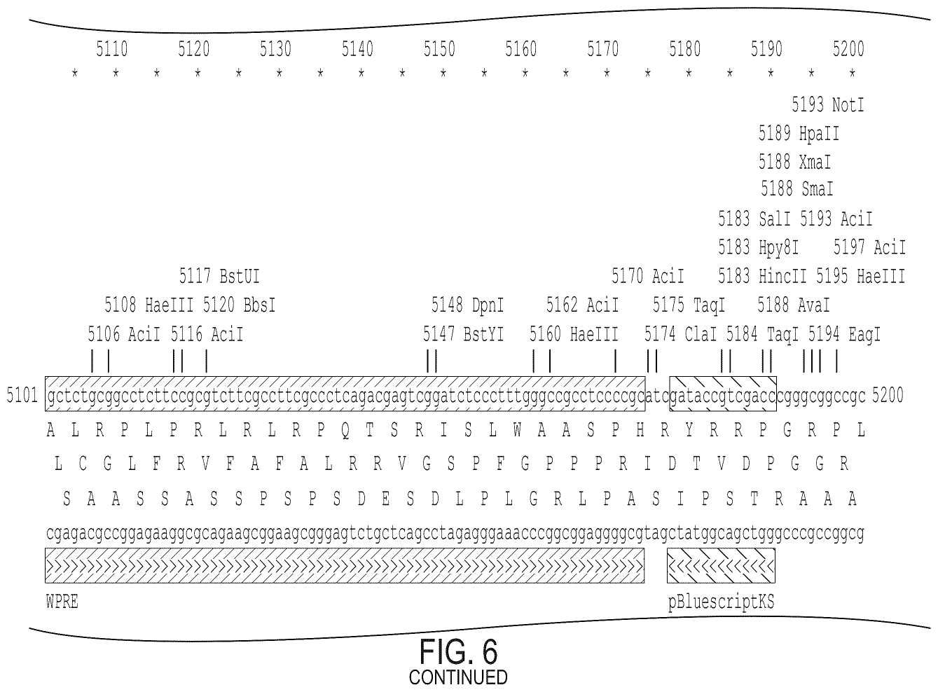

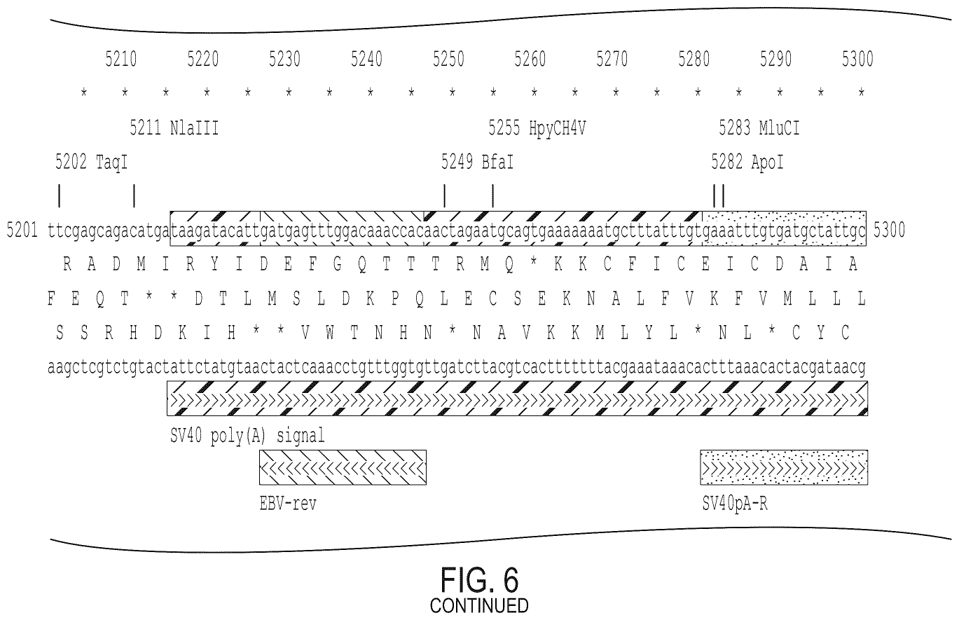

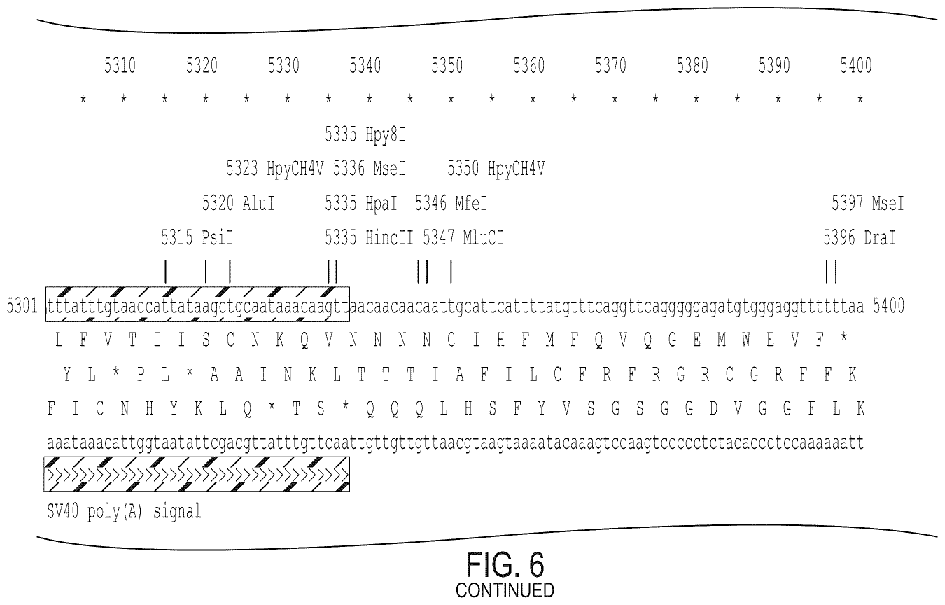

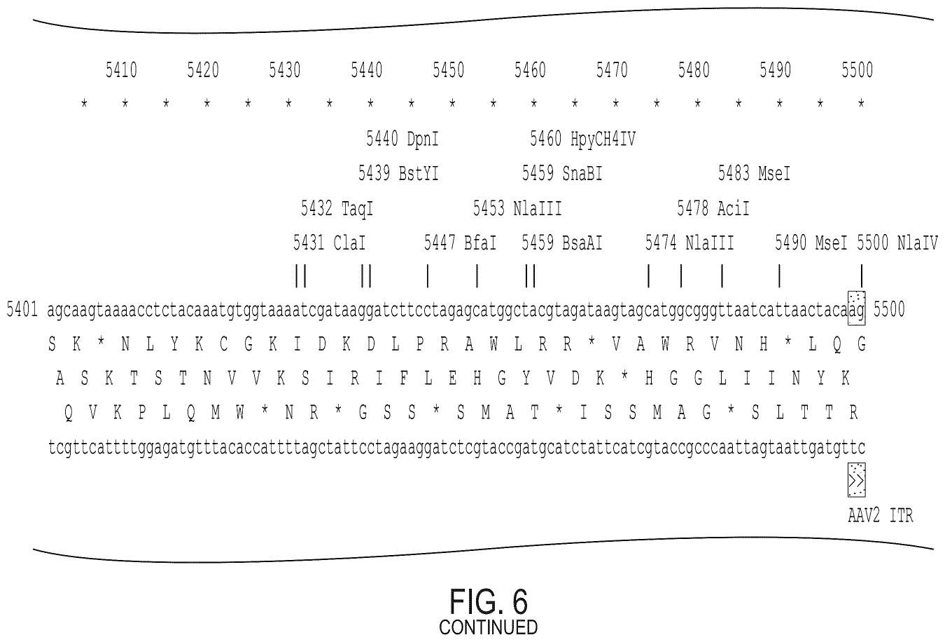

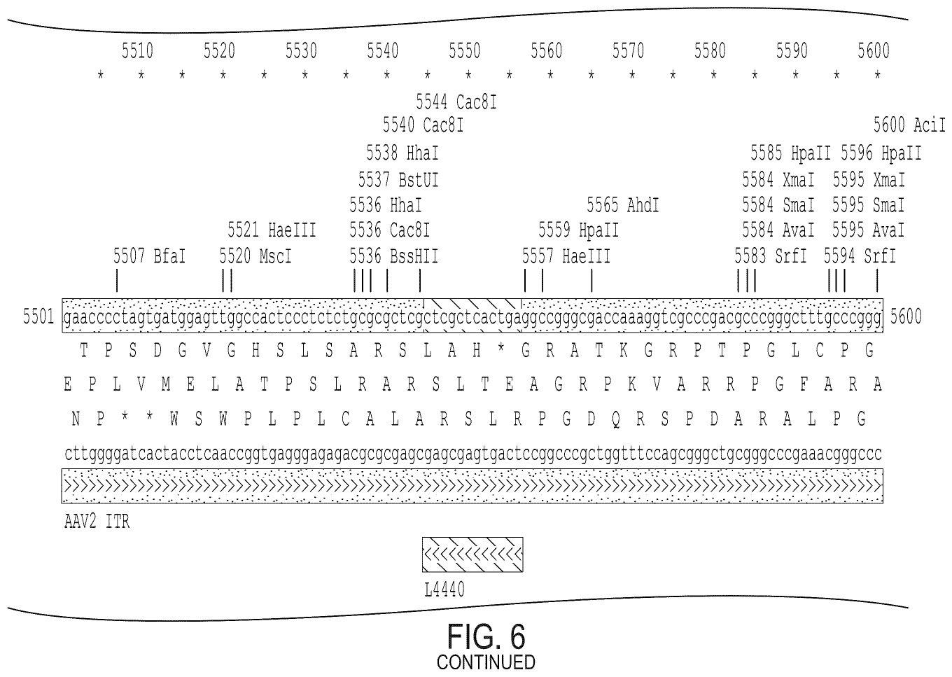

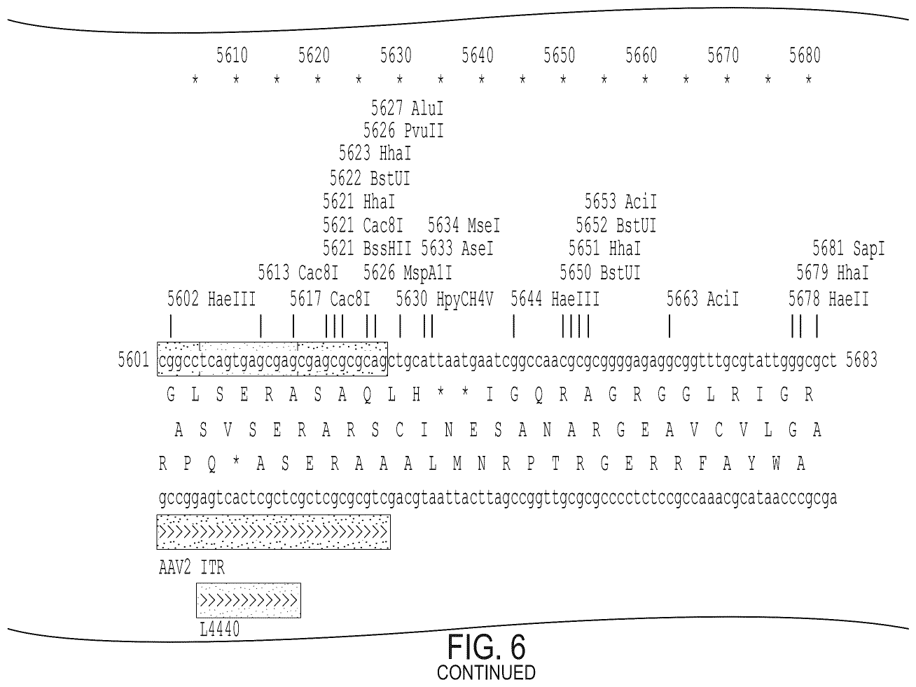

In certain embodiments, the AAV vector contains two inverted terminal repeats (ITRs). In some embodiments, the ITRs are AAV2 ITRs. In some embodiments, the AAV2 ITRs comprise SEQ ID NOs: 4 and 5. In some embodiments, the AAV vector comprises a nucleotide sequence encoding a selectable marker such as an antibiotic resistance gene. One exemplary expression vector is the nucleic acid sequence of SEQ ID NO: 10 provided in FIG. 6, which is an AAV vector comprising the cholinergic enhancer sequence of SEQ ID NO: 1 and an mCherry sequence positioned 3′ to the cholinergic enhancer sequence such that the cholinergic enhancer drives expression of mCherry in cholinergic neurons. Other nucleotide sequences may replace the sequence encoding mCherry with a sequence encoding a polypeptide of interest or an RNA of interest as described herein. Other nucleotide sequences may replace any origin(s) of replication, mini-promoter, chimeric intron, woodchuck hepatitis virus post-transcriptional regulatory element (WPRE), and/or SV40 polyA sequence with other known sequences that perform the same or similar function to these elements which are known in the art.

### Cells and Animals

In another aspect, described herein are cells comprising the nucleic acid, expression cassette, or viral vector as described herein. In certain embodiments, the viral vector is an AAV vector. In some embodiments, the cells may further comprise an AAV Rep gene and an AAV Cap gene. The AAV Rep and Cap genes may be driven by an AAV promoter such as the p19 and p40 promoters, or they may be driven by a heterologous promoter such as a human cytomegalovirus (CMB) immediate-early enhancer and promoter. In some embodiments, the AAV Rep gene and/or AAV Cap gene are AAV2 genes.

In certain embodiments, the cells further comprise AAV helper genes. AAVs require genes from adenovirus to mediate AAV replication and particle production. Helper genes for AAV include the adenovirus E2A, E4, VA, and E1 genes. These helper genes may be transiently express in the cell as a plasmid, or they may be stably integrated in the cell. A cell line commonly used for production of AAV particles is the HEK293 cell line, which contains the adenovirus gene E1. The remaining helper genes are supplied in the form of a helper plasmid. When the AAV vector comprising the nucleotide sequences described herein positioned between two ITRs is expressed in a cell with AAV Rep/Cap genes and the AAV helper genes, the nucleotide sequence positioned between the ITRs is replicated and packaged in an AAV particle to be delivered to target cells such as cholinergic neurons.

Cells comprising these viral vectors may be cultured in any useful media. Cells can be any permissive cell or tissues, which may be derived from mammals, including, but not limited to, cell lines derived from rodent, murine, human, canine, feline, equine, bovine or porcine cell lines. As used herein, a cell or a tissue can include, but is not limited to individual cells, tissues, organs, insect cells, rodent cells, avian cells, mammalian cells, hybridoma cells, primary cells, continuous cell lines, and/or genetically engineered cells. Cell culture media formulations to suitable for culturing cells are known in the art.

An exogenous nucleic acid, for example a nucleic acid comprising SEQ ID NO: 1 or vector containing SEQ ID NO: 1, can be introduced into a cell via a variety of techniques known in the art, for example, but not limited to lipofection, microinjection, calcium phosphate or calcium chloride precipitation, DEAE-dextrin-mediated transfection, or electroporation.

Cells can be primary and secondary cells, which can be obtained from various tissues and include cell types which can be maintained and propagated in culture.

### Polypeptides of Interest and Oligonucleotides of Interest

In certain aspects, described herein are nucleic acids that encode polypeptides of interest and are positioned 3′ of the enhancer sequence such that the enhancer sequence drives expression of the polypeptides of interest. These polypeptides include, but are not limited to, prophylactic polypeptides, therapeutic polypeptides, and gene-editing polypeptides. In certain embodiments, the polypeptide is a neurotrophic factor or neuroprotective factor that allows neurons such as motor neurons to better handle age-related stresses such as oxidative stress, protein misfolding, and DNA damage. In some embodiments, the polypeptides reduce the degradative effects of neurologic disorders. Prophylactic and therapeutic polypeptides may include, but are not limited to, neurotrophic factors such as brain-derived neurotrophic factor (BDNF), glial cell line derived neurotrophic factor (GDNF), insulin-like growth factor 1 (IGF-1), and vascular endothelial growth factors (VEGF). Additional exemplary prophylactic or therapeutic polypeptides that may be encoded are described in R. Chia et al., Novel Genes Associated with Amyotrophic Lateral Sclerosis: Diagnostic and Clinical Implications, Lancet Neurol. 2018 January; 17(1):94-102, D. Amado & B. Davidson, Gene Therapy for ALS: A Review, Molecular Therapy. 2021 December; 29(12): pp. 3345-3358, Fang et al., Gene Therapy in Amyotrophic Lateral Sclerosis, Cells. 2022 July; 11(13): 2066, and US2020/0101138A1, each of which are herein incorporated by reference in their entirety. In some embodiments, the polypeptides of interest is Is11 or Lhx3.

An exemplary gene-editing polypeptide of interest that may be encoded is Cre recombinase. Cre recombinase recognizes repeated DNA sequences called loxP and mediates site-specific deletion of DNA between the two loxP sites, thus “knocking out” the gene between the loxP sites. The Cre-loxP system is a commonly employed system in animal models such as mouse or rat models to temporally or spatially express or knock out genes of interest. If a transgenic mouse expresses a gene of interest flanked by 2 loxP sites, this gene can be knocked out in tissues where Cre recombinase is expressed. Cre expression can be controlled spatially, for example, when Cre recombinase is driven by a tissue specific promoter, and/or temporally, for example, when a nucleotide sequence encoding Cre recombinase is packaged in an AAV particle and delivered to the transgenic mouse at a certain time point.

In certain aspects, described herein are nucleic acids encoding RNAs of interest, which includes, but is not limited to an interfering RNA (iRNA), and variants thereof, that can silence a target gene. An iRNA can down-regulate the expression of a target gene, e.g., SOD1, C9orf72, ATXN2, FUS, and L3MRTL1. For example, L3MBTL1 is a gene that is expressed at higher levels in adult motor neurons and that has been shown to suppress proteasome activity and protein degradation in C. elegans models of ALS. Suppression of this protein may improve the ability of motor neurons to handle misfolded proteins. Additional exemplary genes of interest for ALS are discussed in R. Chia et al., Novel Genes Associated with Amyotrophic Lateral Sclerosis: Diagnostic and Clinical Implications, Lancet Neurol. 2018 January; 17(1):94-102, D. Amado & B. Davidson, Gene Therapy for ALS: A Review, Molecular Therapy. 2021 December; 29(12): pp. 3345-3358, Fang et al., Gene Therapy in Amyotrophic Lateral Sclerosis, Cells. 2022 July; 11(13): 2066, and Lu et al., L3MBTL1 Regulated ALS/FTD-Associated Proteotoxicity and Quality Control, Nat Neurosci. 2019 June; 22(6):875-886, each of which are herein incorporated by reference in their entirety. An iRNA may act by one or more of a number of mechanisms, including post-transcriptional cleavage of a target mRNA sometimes referred to in the art as RNAi, or pre-transcriptional or pre-translational mechanisms. An iRNA can be a double stranded (ds) iRNA. A ds iRNA includes more than one, and in certain embodiments two, strands in which interchain hybridization can form a region of duplex structure. A strand refers to a contiguous sequence of nucleotides (including non-naturally occurring or modified nucleotides). At least one strand can include a region which is sufficiently complementary to a target RNA. Such strand is termed the antisense strand. A second strand comprised in the dsRNA which comprises a region complementary to the antisense strand is termed the sense strand. However, a ds iRNA can also be formed from a single RNA molecule which is, at least partly; self-complementary, forming, e.g., a hairpin or panhandle structure, including a duplex region. In such case, the term strand refers to one of the regions of the RNA molecule that is complementary to another region of the same RNA molecule. Nonlimiting examples of inhibitory RNA include miRNA, siRNA, shRNA, and piRNA.

iRNA as described herein, including ds iRNA and siRNA, can mediate silencing of a gene, e.g., by RNA degradation. For convenience, such RNA is also referred to herein as the RNA to be silenced. Such a gene is also referred to as a target gene. In certain embodiments, the gene to be silenced is SOD1, C9orf72, ATXN2, FUS, or L3MRTL1.

In certain embodiments, the oligonucleotide of interest is a guide RNA (gRNA) or single guide RNA (sgRNA). The CRISPR/Cas9 gene editing technique promotes a new human gene therapy strategy by correcting a defect gene at pre-chosen sites without altering the endogenous regulation of the target gene. This system consists of two key components: Cas9 protein and a guide RNA, e.g., a single guide RNA (sgRNA), as well as a correction template when needed. sgRNA contains two components: a 17-20 nucleotide sequence termed crispr RNA that is complementary to the target DNA region, and a tracr RNA that serves as the binding scaffold for a Cas nuclease. The sgRNA recognizes the target DNA and guides the Cas9 nuclease to the region for editing.

### Treatment of Cholinergic Neurons

In another aspect, described herein is a method of treating neurologic disorders in a subject in need thereof comprising administering to a subject a therapeutically effective amount of an AAV particle comprising the nucleotide sequence at least 70% identical to SEQ ID NO: 1 or a pharmaceutical composition comprising said AAV particle. In certain embodiments, the subject is a mammal. In some embodiments, the subject is a mouse or a rat, and preferably the subject is a human. Neurologic disorders that may preferentially benefit from the disclosures herein include neurologic disorders associated with a defect in cholinergic neurons. Such cholinergic neurons may include basal forebrain cholinergic neurons and lower motor neurons. Such neurologic disorders include, but are not limited to, Amyotrophic Lateral Sclerosis (ALS), Alzheimer's disease, Parkinson's disease, Myasthenia gravis, Huntington's chorea, Spinal Muscular Atrophy, Kennedy Disease, Progressive Muscular Atrophy, and Monomelic Amyotrophy. In some embodiments, the neurologic disorder is Alzheimer's disease. In some embodiments, the neurologic disorder is ALS.

In certain aspects, described herein is a method for rejuvenating spinal motor neurons or increasing motor neuron resistance to ALS pathogens in a subject with ALS. In some embodiments, the method uses an enhancer specific to a subset of motor neurons such as lower motor neurons and is packaged in AAVs. In some embodiments, a subject is administered the AAVs containing the enhancer to induce gene expression or knockdown in spinal motor neurons at different developmental stages. In certain embodiments, the enhancer is a nucleotide sequence at least 70% identical to SEQ ID NO: 1. In some embodiments, the enhancer sequence drives a nucleotide sequence encoding a polypeptide of interest or an oligonucleotide of interest, wherein the nucleotide sequence encoding the polypeptide of interest or oligonucleotide of interest is positioned 3′ to the enhancer sequence. In certain embodiments, the polypeptide of interest or oligonucleotide of interest are the polypeptides and oligonucleotides described above herein.

Cholinergic neurons are neurons that utilize the neurotransmitter acetylcholine, synthesized through activity of the enzyme choline acetyltransferase (ChAT) to send messages. Cholinergic neurons are distributed throughout the central nervous system, including but not limited to the spinal cord, striatum, hindbrain, and basal forebrain. One subset of cholinergic neurons are spinal motor neurons or lower motor neurons. These neurons are involved in sensory, autonomic and motor control in the spinal cord. Degeneration of these neurons is a characteristic of ALS. Another subset of cholinergic neurons are basal forebrain cholinergic neurons. These neurons are critical for a range of cognitive functions, and loss of these neurons is characteristic of Alzheimer's Disease.

In certain embodiments, the AAV particles are administered by injection, which may include, but is not limited to, intravenous, intracerebroventricular, intrathecal, intraparenchymal, intramuscular, or intraperitoneal injection.

Therapeutically effective amount refers to an amount that is effective for preventing, ameliorating, treating or delaying the onset of a disease or condition. The pharmaceutical compositions (e.g. comprising an AAV particle described herein) of the inventions can be administered to any animal that can experience the beneficial effects of the agents of the invention. Such animals include humans and non-humans.

Routes of administration and dosages of effective amounts of the pharmaceutical compositions (e.g. comprising an AAV particle described herein) are also disclosed. The agents of the present invention can be administered in combination with other pharmaceutical agents in a variety of protocols for effective treatment of disease.

Pharmaceutical compositions (e.g. comprising an AAV particle described herein) are administered to a subject in a manner known in the art. The dosage administered will be dependent upon the age, health, and weight of the recipient, kind of concurrent treatment, if any, frequency of treatment, and the nature of the effect desired. One may administer the pharmaceutical compositions in a local rather than systemic manner, for example, via injection of directly into the desired target site, often in a depot or sustained release formulation.

One of ordinary skill in the art will appreciate that a method of administering pharmaceutically effective amounts of the pharmaceutical compositions (e.g. comprising an AAV particle described herein) to a patient in need thereof, can be determined empirically, or by standards currently recognized in the medical arts. The AAV particles can be administered to a patient as pharmaceutical compositions in combination with one or more pharmaceutically acceptable excipients. It will be understood that, when administered to a human patient, the total daily usage of the agents of the pharmaceutical compositions (e.g. comprising an AAV particle described herein) will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

### Research Applications

The compositions and methods described herein may also be utilized for research purposes. In one aspect, described herein is a method of studying cholinergic activity in the central nervous system or motor neuron activity in the peripheral nervous system comprising administering to a subject an AAV particle comprising the nucleotide sequence at least 70% identical to SEQ ID NO: 1 and any polypeptide or oligonucleotide of interest. In certain embodiments, the subject is a mammal, in some embodiments, the subject is a mouse or a rat, and in certain embodiments, the subject is a human. In some embodiments, the method comprises determining cholinergic activity. In some embodiments, the method comprises determining cholinergic activity at one or more time points before administration of the AAV particle. In some embodiments, the method comprises determining cholinergic activity at one or more time points after administration of the AAV particle. In some embodiments, the method comprises comparing cholinergic activity between one or more time points before and after administration of the AAV particle.

In certain embodiments, the AAV particles are administered by injection, which may include, but is not limited to, intravenous, intracerebroventricular, intrathecal, intraparenchymal, intramuscular, or intraperitoneal injection.

### Enhancer to Control Gene Expression in a Subset of Cholinergic Neurons

A commonly studied promoter to control gene expression in cholinergic neurons is the HB9 promoter described in U.S. Pat. No. 7,632,679 and S. Arber et al., Requirement for the Homeobox Gene Hb9 in the Consolidation of Motor Neuron Identity, Neuron. 1999 August; 23(4):659-74, each of which are herein incorporated by reference in their entirety. Key disadvantages of the HB9 promoter include its large (9.5 kb) size, hindering its compatibility with viral packaging, and the fact that it is only active during early postnatal stages. Various groups have attempted to identify shorter segments of the HB9 promoter, but these enhancers control expression only in nascent embryonic motor neurons. There is currently an effort in the field to develop a new enhancer that can be used to control gene expression specifically in a subset of cholinergic neurons. These cholinergic neurons include motor neurons affected in ALS as well as basal forebrain cholinergic neurons affected in AD and other diseases. The cholinergic enhancer disclosed in SEQ ID NO: 1 was identified by mapping accessible chromatin regions around the CHAT gene. Importantly, this enhancer is much smaller (1 kb) than the HB9 promoter. This makes the cholinergic enhancer fully compatible with viral packaging. Additionally, this regulatory element can be used to drive expression in cholinergic neurons at all ages from early postnatal to adulthood.

SEQUENCES
SEQ ID NO: 1 depicts the nucleotide sequence of the enhancer dis-
closed herein:
CAGTGAGCTTCATTATCACCTAACAGCTTCAGAGTGGGTGGTGGGTTTTGGA
TGACAACCTTTCTTCTCATTTTATTCAGTGGCCACACCGTGGCCTTAGTCTGA
TAAACCAAAAACCTGCTCCATTATGAATCAGTGCTGTGGGGAGTGGGTAGAG
AGTGTGAAGTTCTGGGGTGGGGGAGTCTGGAGAGAGGGTGGGAGCAGCCAT
TCTGCAGCAGTGCCTTCTTGGGGTCATGGGTCTGTAGGTGCTGCTGTGGAGG
GAGAGATCAGCCTATTCTGGCTTCATTTCTGAGCTGCAAACTGCCTGGGTGTC
TGGAGAAGCAGGTTGGCGTGGTGGTTAGCAGTGCGTGGGCGGGGTTGCCCG
CTCTTGATTTATGATTTCTTTGTCTCTGTGGAAGCACTTAAGTGCAGGCTTTA
GTTCCAATGACACTCAGGAGCCTCTGGATTCCAGCACTGGGGATGGGGGTGG
GGTAGAACGTTCTCAGGCCTCACCAACCCCTCCCCTGTGTGCTGCCTTTGGGA
GAGTCCCAAGGCTTCAGCATTACTTAATTAATTAGGCCTCTACTGCTACATAG
GCTCAGATTCAAAAGAACAGAGTGGCCCACGTCAGCCATTCCCGGAAAAGT
CTGATGGCTGGAAGCCAGAGGACTATGTGTCTGCCTTGCTGCCCTTGGCCAG
CCCATCCTGAATGCCCAGACTCGGACAATGGAGTAGGTACAGAAGGGTAAA
GACAGTGTCTTCTGTACCAGTAAGTGGGCCCTGATCTGCTCTCTACAGCTTCC
AGAGAAAGGGCCTGGCCAATGAGCGGCCTTTTGAGTAGCAGATACCTCACAT
GCATTCTGATAGAAAGCCTGGCCCCAGATCACTGTGACTTTAGCCCTCAGGT
TTCTTTTGCACTTCAATTCAATGACTTCTTGAGGTTCATTTCCCTCTCCAAGAT
TTGCCACAGACCAGTGGTTCTCAACCTGTGGGTCACACCTCCTTTGGGGAAA
TTGAATGA
SEQ ID NO: 2 depicts the nucleotide sequence of the ampicillin
resistance gene disclosed herein:
ATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAG
ATCAGTTGGGTGCACGAGTGGGTTACATCGAACTGGATCTCAACAGCGGTAA
GATCCTTGAGAGTTTTCGCCCCGAAGAACGTTTTCCAATGATGAGCACTTTTA
AAGTTCTGCTATGTGGCGCGGTATTATCCCGTATTGACGCCGGGCAAGAGCA
ACTCGGTCGCCGCATACACTATTCTCAGAATGACTTGGTTGAGTACTCACCAG
TCACAGAAAAGCATCTTACGGATGGCATGACAGTAAGAGAATTATGCAGTGC
TGCCATAACCATGAGTGATAACACTGCGGCCAACTTACTTCTGACAACGATC
GGAGGACCGAAGGAGCTAACCGCTTTTTTGCACAACATGGGGGATCATGTAA
CTCGCCTTGATCGTTGGGAACCGGAGCTGAATGAAGCCATACCAAACGACGA
GCGTGACACCACGATGCCTGTAGCAATGGCAACAACGTTGCGCAAACTATTA
ACTGGCGAACTACTTACTCTAGCTTCCCGGCAACAATTAATAGACTGGATGG
AGGCGGATAAAGTTGCAGGACCACTTCTGCGCTCGGCCCTTCCGGCTGGCTG
GTTTATTGCTGATAAATCTGGAGCCGGTGAGCGTGGGTCTCGCGGTATCATTG
CAGCACTGGGGCCAGATGGTAAGCCCTCCCGTATCGTAGTTATCTACACGAC
GGGGAGTCAGGCAACTATGGATGAACGAAATAGACAGATCGCTGAGATAGG
TGCCTCACTGATTAAGCATTGGTAA
SEQ ID NO: 3 depicts the amino acid sequence encoded by the
ampicillin resistance gene disclosed herein:
MSIQHFRVALIPFFAAFCLPVFAHPETLVKVKDAEDQLGARVGYIELDLNSGKILE
SFRPEERFPMMSTFKVLLCGAVLSRIDAGQEQLGRRIHYSQNDLVEYSPVTEKHL
TDGMTVRELCSAAITMSDNTAANLLLTTIGGPKELTAFLHNMGDHVTRLDRWEP
ELNEAIPNDERDTTMPVAMATTLRKLLTGELLTLASRQQLIDWMEADKVAGPLL
RSALPAGWFIADKSGAGERGSRGIIAALGPDGKPSRIVVIYTTGSQATMDERNRQI
AEIGASLIKHW-
SEQ ID NO: 4 depicts the nucleotide sequence of an exemplary
AAV2 ITR disclosed herein:
CTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGT
GGCCAACTCCATCACTAGGGGTTCCT
SEQ ID NO: 5 depicts the nucleotide sequence of an exemplary
AAV2 ITR disclosed herein:
AGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGG
CCTCAGTGAGCGAGCGAGCGCGCAG
SEQ ID NO: 6 depicts the nucleotide sequence of an exemplary
promoter disclosed herein:
TAGAGGGTATATAATGGAAGCTCGACTTCCAG
SEQ ID NO: 7 depicts the nucleotide sequence of an exemplary
intron disclosed herein:
GTAAGTATCAAGGTTACAAGACAGGTTTAAGGAGACCAATAGAAACTGGGCT
TGTCGAGACAGAGAAGACTCTTGCGTTTCTGATAGGCACCTATTGGTCTTACT
GACATCCACTTTGCCTTTCTCTCCACAG
SEQ ID NO: 8 depicts the nucleotide sequence of an exemplary
woodchuck hepatitis virus post-transcriptional regulatory element
disclosed herein:
AATCAACCTCTGGATTACAAAATTTGTGAAAGATTGACTGGTATTCTTAACTA
TGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGTGCA
CTGTGTTTGCTGACGCAACCCCCACTGGTTGGGGCATTGCCACCACCTGTCAG
CTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGT
GTTGCCACCTGGATTCTGCGCGGGACGTCCTTCTGCTACGTCCCTTCGGCCCT
CAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCC
CCGC
SEQ ID NO: 9 depicts the nucleotide sequence of an exemplary
polyA sequence disclosed herein:
TAAGATACATTGATGAGTTTGGACAAACCACAACTAGAATGCAGTGAAAAAA
ATGCTTTATTTGTGAAATTTGTGATGCTATTGCTTTATTTGTAACCATTATAAG
CTGCAATAAACAAGTT

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Functional Validation of the Cholinerigic Enhancer Packaged in AAVs

A reporter construct (SEQ ID NO: 10) was used to generate AAVs and the AAVs were injected into early postnatal mice. The reporter construct packaged in the AAVs comprises a nucleotide sequence encoding the cholinergic enhancer (SEQ ID NO: 1) and a nucleotide sequence encoding the fluorescent protein mCherry positioned 3′ of the cholinergic enhancer. Expression of the reporter could be seen in cholinergic neurons in the spinal cord.

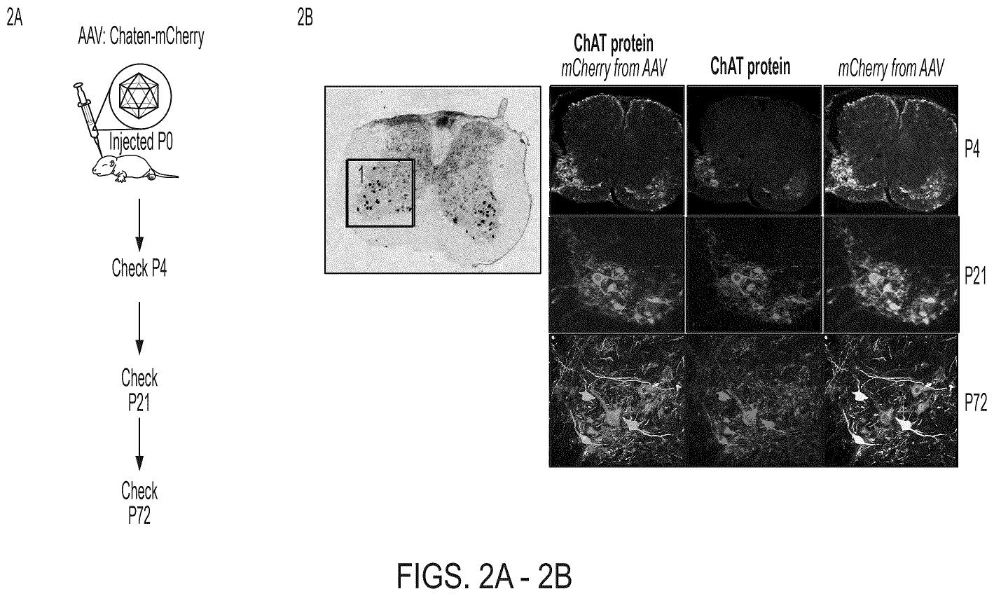

The experimental design for this example is depicted in FIG. 2A. Briefly, mice were injected at PO (day of birth) with an AAV particle comprising the cholinergic enhancer and the fluorescent protein mCherry. Spinal cords of these mice were then collected at various post-injection time points, and immunofluorescence was performed to detect mCherry expression. Images in FIG. 2B are of whole spinal cords or region 1 which contains spinal motor neurons. mCherry was visible in motor neurons as early as 4 days after injection and persisted still at 72 days after injection, which was the latest developmental stage checked. Motor neurons were labeled with ChAT protein.

### Example 2—Functional Validation of the Enhancer Packaged in AAVs Driving Expression of Either Is1 or Lhx3

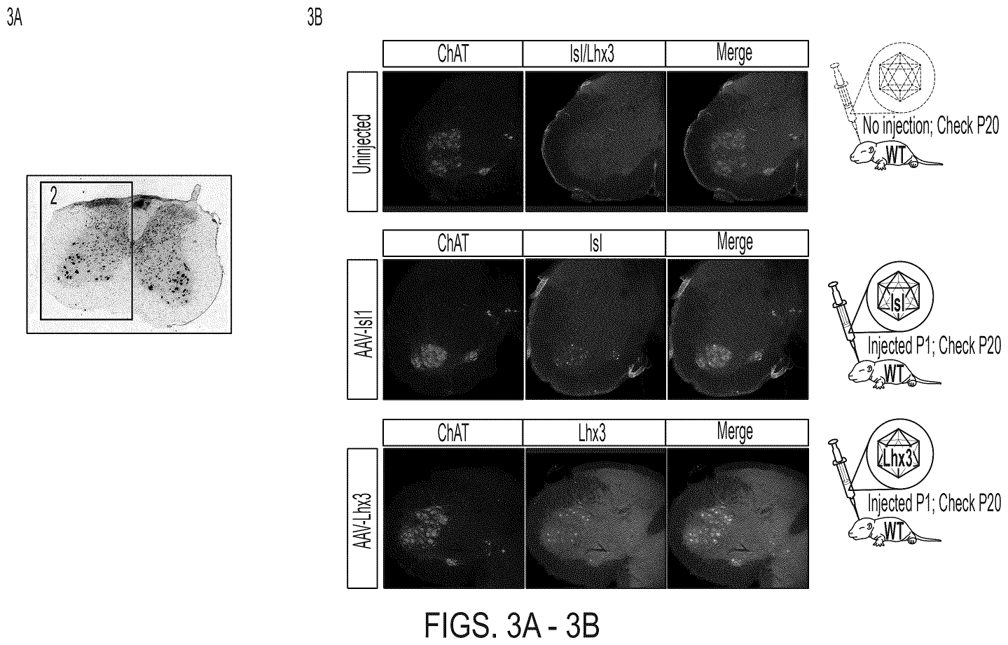

In this experiment, mice were injected at PO (day of birth) with an AAV virus that drove the expression of either Is11 or Lhx3, two transcription factors important in the development of motor neurons. Images in FIG. 3B are of spinal cord or region 2. Both transcription factors were detected in motor neurons of injected mice 20 days after injection.

### Example 3—Functional Validation in Juvenile Mice of the Cholinerigic Enhancer Packaged in AAVs

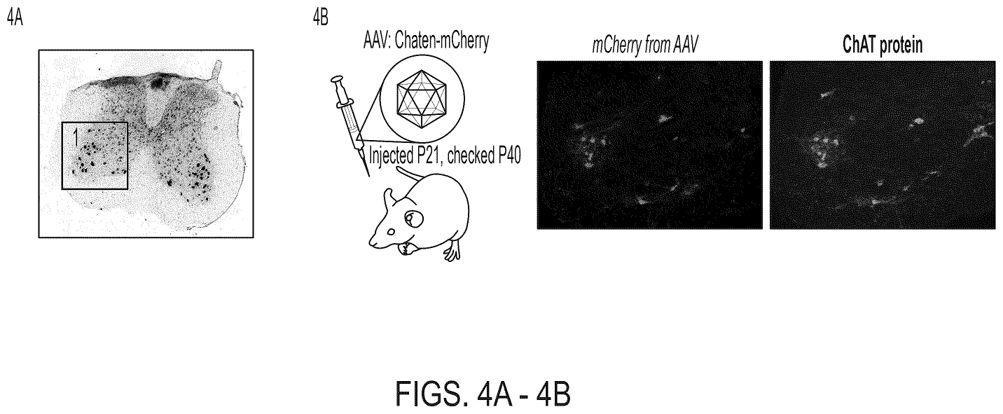

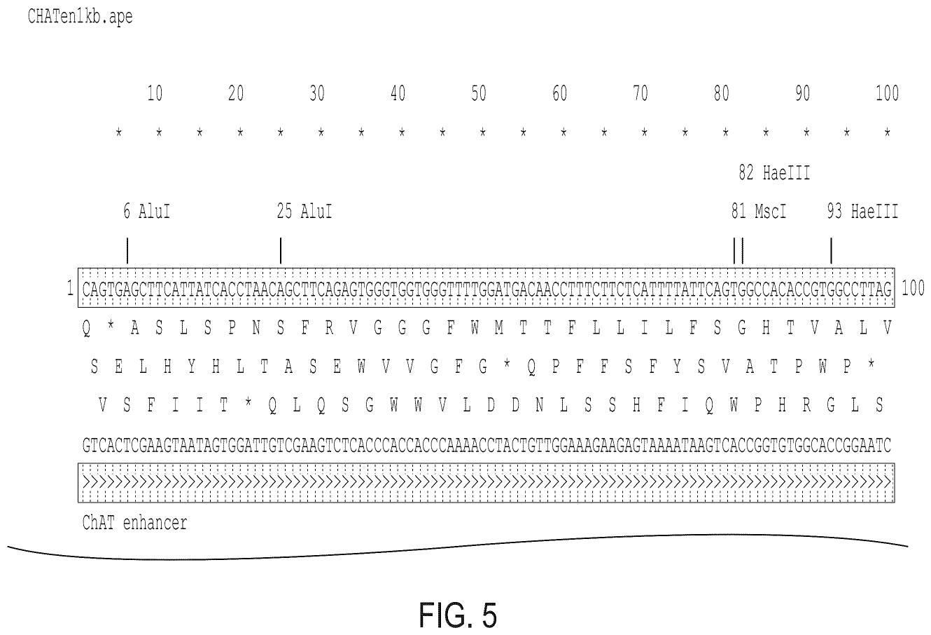

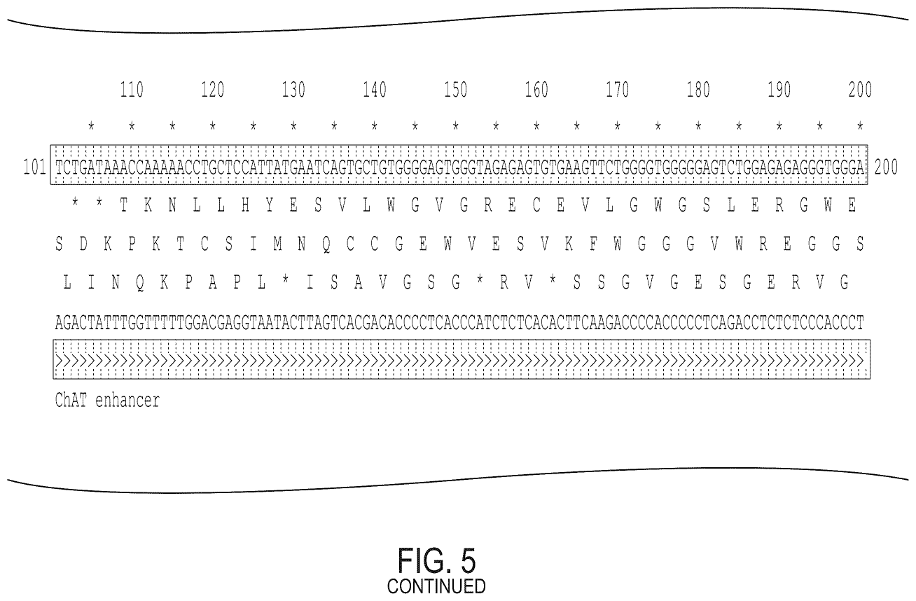

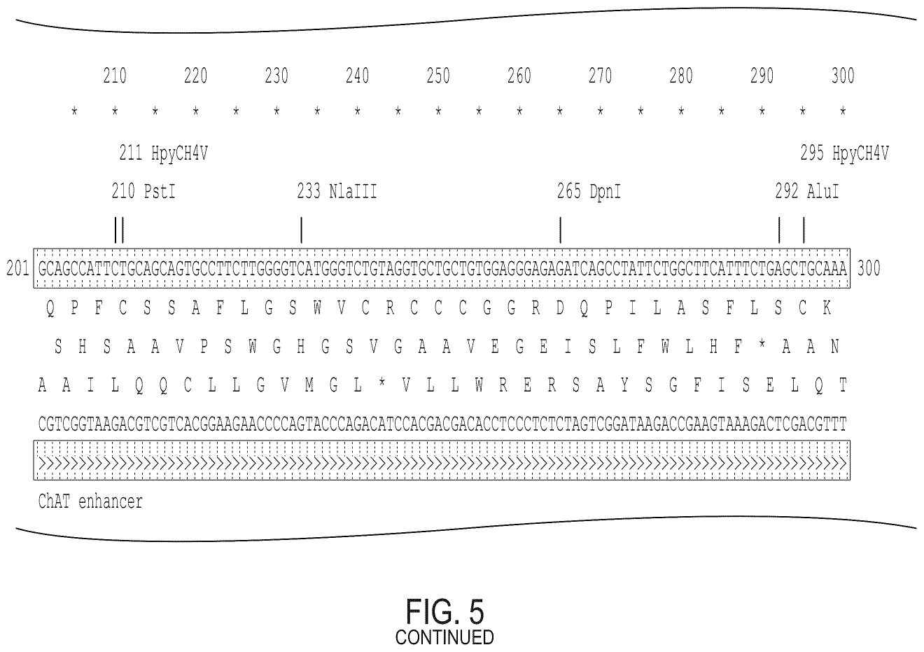

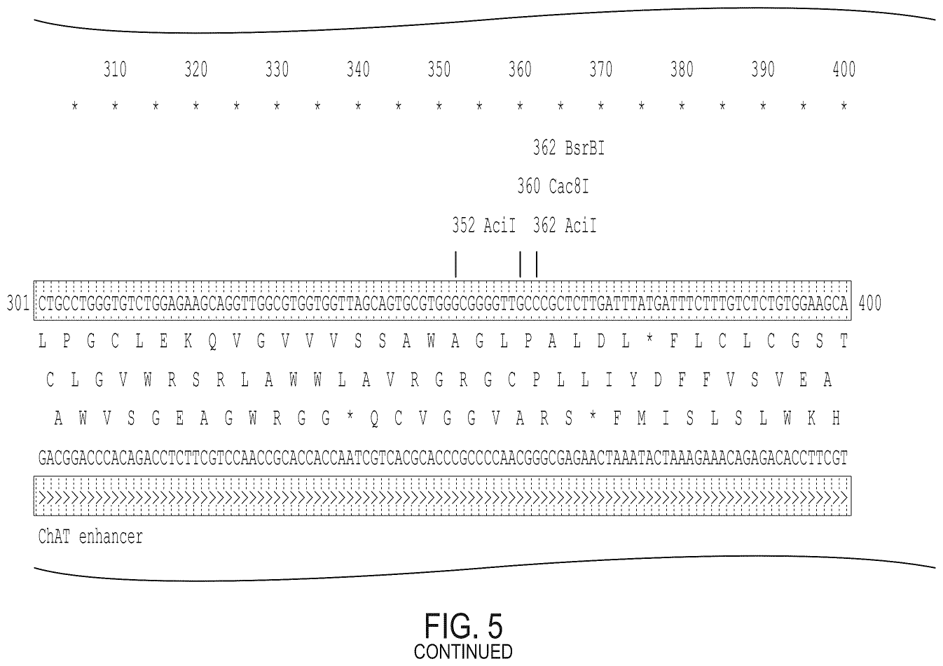

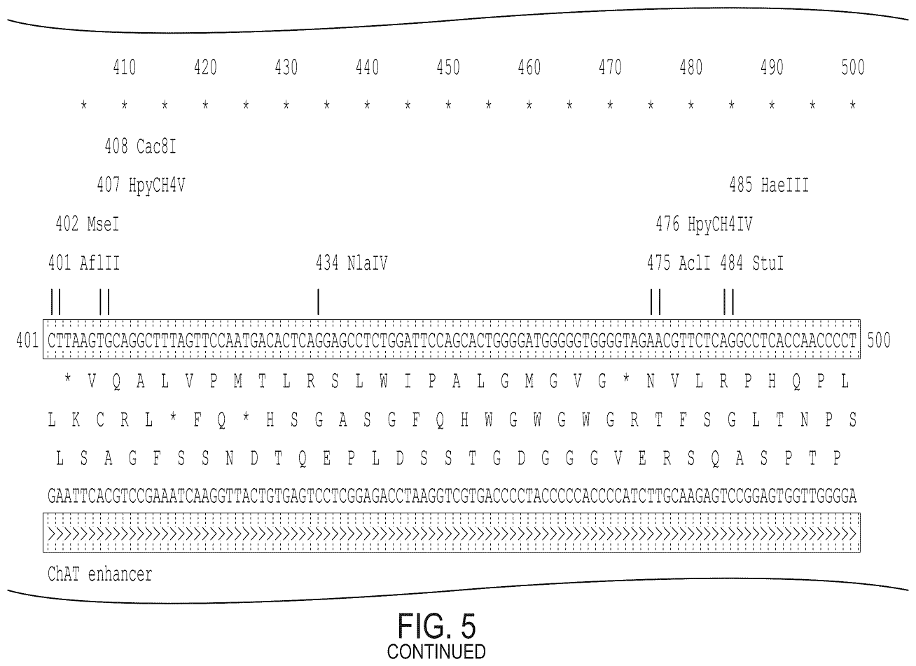

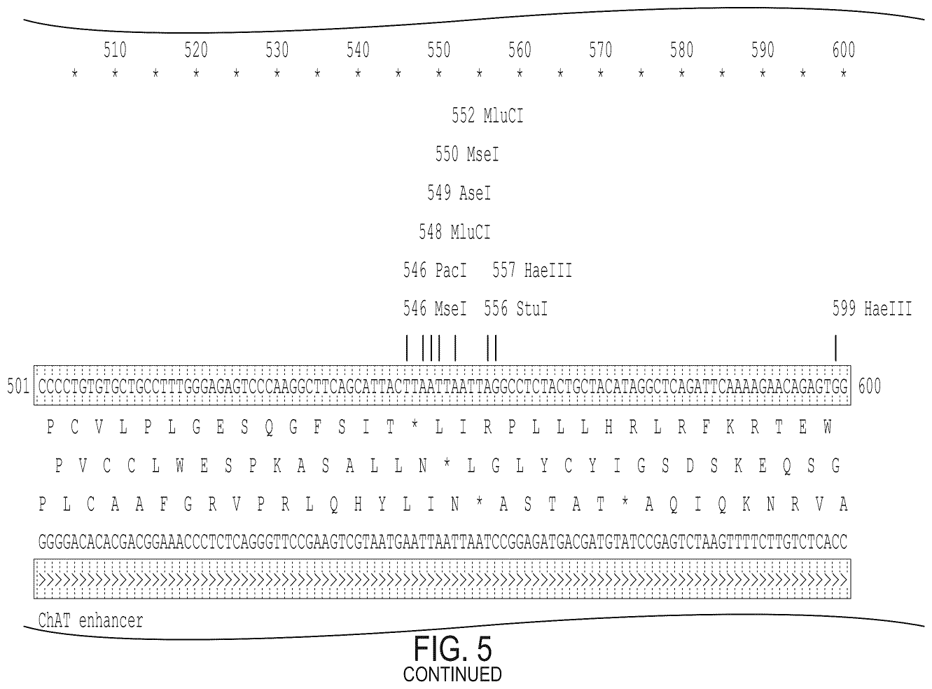

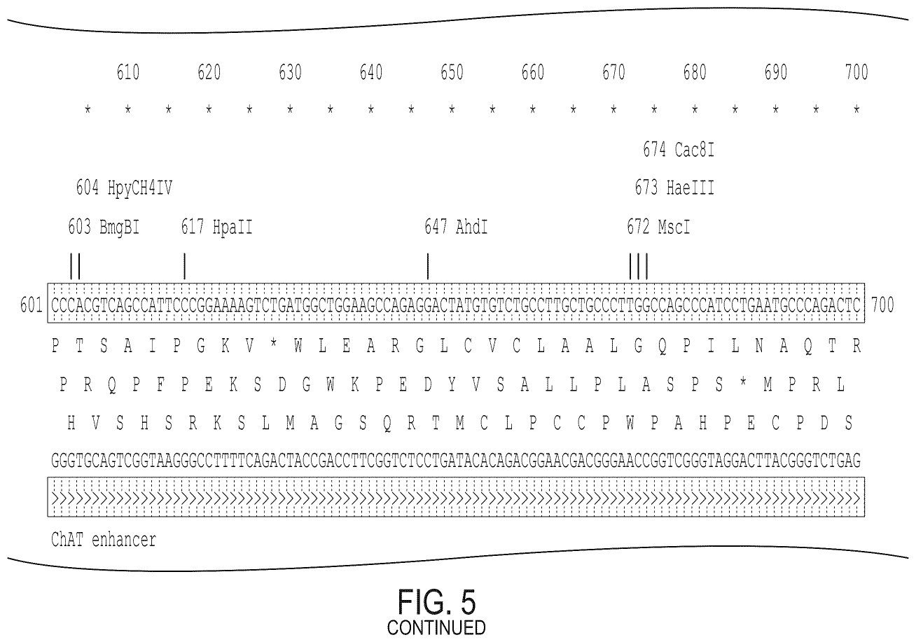

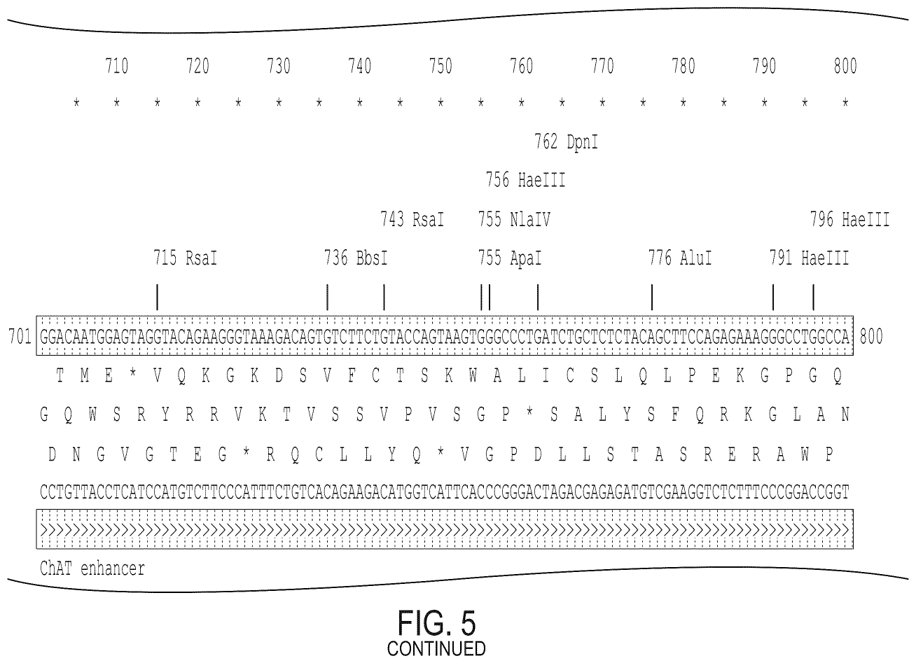

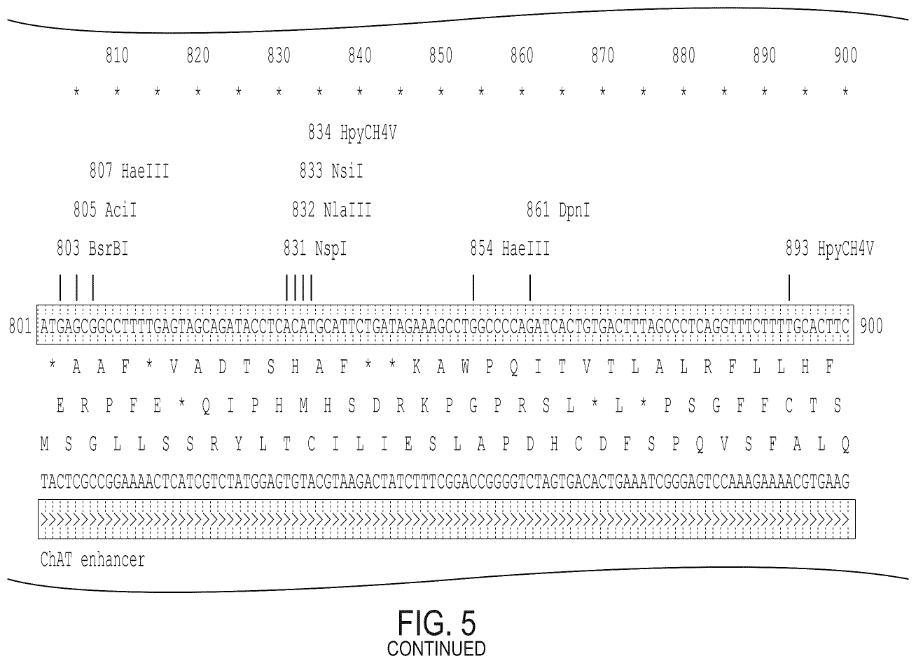

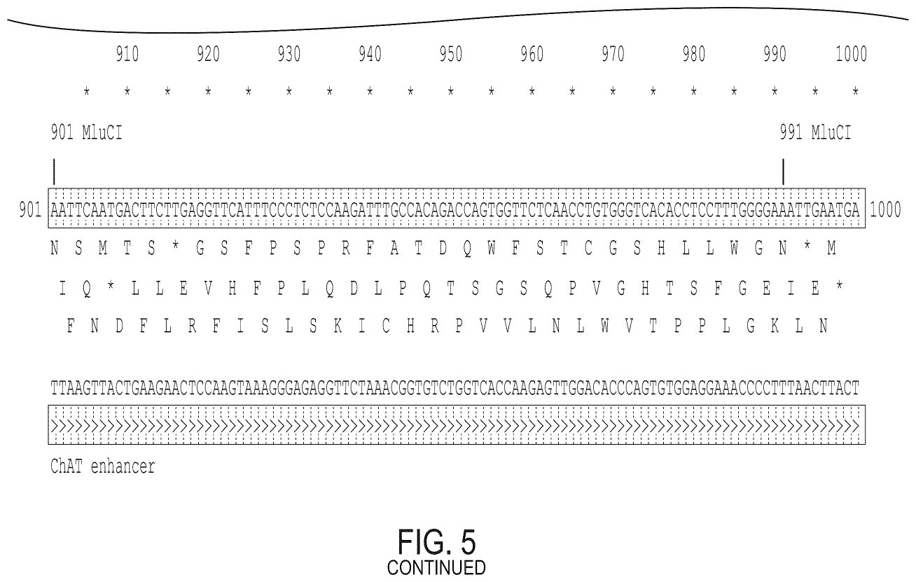

In this experiment, mice were injected at 3 weeks after birth (P21) with an AAV virus that drove expression of fluorescent protein mCherry. Images in FIG. 4B are of region 1 which contains spinal motor neurons. Area 1 sections were imaged 2.5 weeks after injection (P40), and mCherry was detected in motor neurons, which were labeled with ChAT protein.

### Example 4—AAV-Mediated Gene Delivery of Cholinergic Enhancer and Inhibitory RNA

The cholinergic enhancer described in SEQ ID NO: 1 is packaged with an inhibitory RNA into an AAV particle. The inhibitory siRNA is targeted against a gene that is commonly overexpressed in ALS. The AAV particle is injected into a patient suffering from ALS, and the cholinergic enhancer drives expression of the siRNA in spinal motor neurons, inhibiting expression of the overexpressed gene.

### Example 5—AAV-Mediated Gene Delivery of Cholinergic Enhancer and Polypeptide of Interest

The cholinergic enhancer described in SEQ ID NO: 1 is packaged with a nucleotide sequence encoding a polypeptide of interest as described above herein into an AAV particle. The polypeptide of interest is one that is either 1) downregulated in ALS, or 2) is a neurotrophic factor or a neuroprotective factor. The AAV particle is injected into a patient suffering from ALS, and the cholinergic enhancer drives expression of the nucleotide sequence encoding the polypeptide of interest in spinal motor neurons.

1. A nucleic acid comprising: an enhancer sequence comprising a nucleotide sequence at least 70% identical to SEQ ID NO: 1; a nucleotide sequence encoding a polypeptide of interest or an oligonucleotide of interest, wherein the nucleotide sequence encoding the polypeptide of interest or oligonucleotide of interest is positioned 3′ to the enhancer sequence; and a promoter sequence positioned between the enhancer sequence and the nucleotide sequence of interest.

2. The nucleic acid of any of claim 1, wherein the cholinergic neuron is a motor neuron, a lower motor neuron, or a basal forebrain cholinergic neuron.

3. The nucleic acid of any of claim 1, comprising a nucleotide sequence encoding a polypeptide of interest, wherein the polypeptide of interest is a neurotrophic factor or a neuroprotective factor.

4. The nucleic acid of any of claim 1, comprising a nucleotide sequence encoding a polypeptide of interest, wherein the polypeptide of interest is brain-derived neurotrophic factor (BDNF), glial cell line derived neurotrophic factor (GDNF), insulin-like growth factor 1 (IGF-1), or vascular endothelial growth factors (VEGF).

5. The nucleic acid of any of claim 1, wherein the polypeptide of interest is a Cre recombinase.

6. The nucleic acid of any of claim 1, comprising a nucleotide sequence encoding an oligonucleotide of interest, wherein the oligonucleotide of interest is a miRNA, a siRNA, a shRNA, or a piRNA.

7. The nucleic acid of claim 6, wherein the oligonucleotide of interest mediates silencing of SOD1, C9orf72, ATXN2, FUS, or L3MRTL1.

8. The nucleic acid of claim 1, comprising a nucleotide sequence encoding an oligonucleotide of interest, wherein the oligonucleotide of interest is a CRISPR/Cas9 guide RNA or single guide RNA for gene editing.

9. The nucleic acid of any of claim 1, further comprising a nucleotide sequence encoding an antibiotic resistance gene.

10. The nucleic acid of claim 1, further comprising a nucleotide sequence of an intron sequence positioned between the enhancer sequence and the nucleotide sequence encoding the polypeptide of interest or an oligonucleotide of interest.

11. The nucleic acid of claim 1, comprising a nucleotide sequence encoding a polypeptide of interest and further comprising a nucleotide sequence of a polyA sequence positioned 3′ to the nucleotide sequence encoding the polypeptide of interest

12. The nucleic acid of any of claim 1, wherein the enhancer sequence is at least 80%, identical to SEQ ID NO: 1.

13. The nucleic acid of any of claim 1, wherein the enhancer sequence is at least 90% identical to SEQ ID NO: 1.

14. The nucleic acid of claim 1, wherein the enhancer sequence comprises SEQ ID NO: 1.

15. The nucleic acid of claim 1, wherein the enhancer sequence consists of SEQ ID NO: 1.

16. An AAV vector comprising the nucleic acid of claim 1, wherein the nucleic acid is positioned between a first inverted terminal repeat (ITR) of the AAV vector and a second inverted terminal repeat (ITR) of the AAV vector.

17. An AAV particle comprising a nucleic acid comprising: an enhancer sequence comprising a nucleotide sequence at least 70% identical to SEQ ID NO: 1; a nucleotide sequence encoding a polypeptide of interest or an oligonucleotide of interest, wherein the nucleotide sequence encoding the polypeptide of interest or oligonucleotide of interest is positioned 3′ to the enhancer sequence; and a promoter sequence positioned between the enhancer sequence and the nucleotide sequence of interest, wherein the nucleic acid is positioned between a first AAV inverted terminal repeat (ITR) and a second AAV ITR.

18. The AAV particle of claim 17, wherein the AAV particle is an AAV2 particle.

19. A cell comprising the nucleic acid of claim 1.

20. The cell of claim 19, further comprising nucleotide sequences encoding an AAV Rep gene, an AAV Cap gene, and one or more AAV helper genes.

21. A method of studying cholinergic activity in the central nervous system or motor neuron activity in the peripheral nervous system of a mammal comprising administering the AAV particle of claim 17.

22. A method of treating a neurologic disorder in a subject in need thereof comprising administering a therapeutically effective amount of the AAV particle of claim 17 to the subject, wherein the neurological disorder is Amyotrophic Lateral Sclerosis, Alzheimer's disease, Parkinson's disease, Myasthenia gravis, Huntington's chorea, Spinal Muscular Atrophy, Kennedy Disease, Progressive Muscular Atrophy, or Monomelic Amyotrophy.

23. A method of gene editing a cholinergic neuron comprising administering the AAV particle of claim 17 to a subject, wherein the cholinergic neuron is a motor neuron, a lower motor neuron, or a basal forebrain cholinergic neuron.

24. A pharmaceutical composition comprising the AAV particle of claim 17.


SEQUENCE LISTING (224 sequences)

SEQ ID NO: 1 - DNA, 1000 bp, unidentified
  source 1..1000: mol_type = unassigned DNA
cagtgagcttcattatcacctaacagcttcagagtgggtggtgggttttggatgacaacctttcttctcattttattcagtggccacaccgtggccttagtctgataaaccaaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaacctgtgggtcacacctcctttggggaaattgaatga

SEQ ID NO: 2 - DNA, 861 bp, unidentified
  source 1..861: mol_type = unassigned DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 3 - protein, 286 aa, unidentified
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 4 - DNA, 130 bp, unidentified
  source 1..130: mol_type = unassigned DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 130 bp, unidentified
  source 1..130: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 32 bp, unidentified
  source 1..32: mol_type = unassigned DNA
tagagggtatataatggaagctcgacttccag

SEQ ID NO: 7 - DNA, 133 bp, unidentified
  source 1..133: mol_type = unassigned DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 8 - DNA, 589 bp, unidentified
  source 1..589: mol_type = unassigned DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 9 - DNA, 122 bp, unidentified
  source 1..122: mol_type = unassigned DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 10 - DNA, 5683 bp, synthetic construct
  source 1..5683: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaggtacccagtgagcttcattatcacctaacagcttcagagtgggtggtgggttttggatgacaacctttcttctcattttattcagtggccacaccgtggccttagtctgataaaccaaaaacctgctccattatgaatcagtgctgtggggagtgggtagagagtgtgaagttctggggtgggggagtctggagagagggtgggagcagccattctgcagcagtgccttcttggggtcatgggtctgtaggtgctgctgtggagggagagatcagcctattctggcttcatttctgagctgcaaactgcctgggtgtctggagaagcaggttggcgtggtggttagcagtgcgtgggcggggttgcccgctcttgatttatgatttctttgtctctgtggaagcacttaagtgcaggctttagttccaatgacactcaggagcctctggattccagcactggggatgggggtggggtagaacgttctcaggcctcaccaacccctcccctgtgtgctgcctttgggagagtcccaaggcttcagcattacttaattaattaggcctctactgctacataggctcagattcaaaagaacagagtggcccacgtcagccattcccggaaaagtctgatggctggaagccagaggactatgtgtctgccttgctgcccttggccagcccatcctgaatgcccagactcggacaatggagtaggtacagaagggtaaagacagtgtcttctgtaccagtaagtgggccctgatctgctctctacagcttccagagaaagggcctggccaatgagcggccttttgagtagcagatacctcacatgcattctgatagaaagcctggccccagatcactgtgactttagccctcaggtttcttttgcacttcaattcaatgacttcttgaggttcatttccctctccaagatttgccacagaccagtggttctcaacctgtgggtcacacctcctttggggaaattgaatgaaccggtctagagggtatataatggaagctcgacttccaggtaagtatcaaggttacaagacaggtttaaggagaccaatagaaactgggcttgtcgagacagagaagactcttgcgtttctgataggcacctattggtcttactgacatccactttgcctttctctccacagggcaaagaattggatccgccaccatggtgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 11 - protein, 32 aa, unidentified
ASLSPNSFRVGGGFWMTTFLLILFSGHTVALV

SEQ ID NO: 12 - protein, 89 aa, unidentified
TKNLLHYESVLWGVGRECEVLGWGSLERGWEQPFCSSAFLGSWVCRCCCGGRDQPILASFLSCKLPGCLEKQVGVVVSSAWAGLPALDL

SEQ ID NO: 13 - protein, 8 aa, unidentified
FLCLCGST

SEQ ID NO: 14 - protein, 22 aa, unidentified
VQALVPMTLRSLWIPALGMGVG

SEQ ID NO: 15 - protein, 24 aa, unidentified
NVLRPHQPLPCVLPLGESQGFSIT

SEQ ID NO: 16 - protein, 26 aa, unidentified
LIRPLLLHRLRFKRTEWPTSAIPGKV

SEQ ID NO: 17 - protein, 27 aa, unidentified
WLEARGLCVCLAALGQPILNAQTRTME

SEQ ID NO: 18 - protein, 29 aa, unidentified
VQKGKDSVFCTSKWALICSLQLPEKGPGQ

SEQ ID NO: 19 - protein, 8 aa, unidentified
VADTSHAF

SEQ ID NO: 20 - protein, 23 aa, unidentified
KAWPQITVTLALRFLLHFNSMTS

SEQ ID NO: 21 - protein, 25 aa, unidentified
GSFPSPRFATDQWFSTCGSHLLWGN

SEQ ID NO: 22 - protein, 17 aa, unidentified
SELHYHLTASEWVVGFG

SEQ ID NO: 23 - protein, 14 aa, unidentified
QPFFSFYSVATPWP

SEQ ID NO: 24 - protein, 63 aa, unidentified
SDKPKTCSIMNQCCGEWVESVKFWGGGVWREGGSSHSAAVPSWGHGSVGAAVEGEISLFWLHF

SEQ ID NO: 25 - protein, 41 aa, unidentified
AANCLGVWRSRLAWWLAVRGRGCPLLIYDFFVSVEALKCRL

SEQ ID NO: 26 - protein, 41 aa, unidentified
HSGASGFQHWGWGWGRTFSGLTNPSPVCCLWESPKASALLN

SEQ ID NO: 27 - protein, 44 aa, unidentified
LGLYCYIGSDSKEQSGPRQPFPEKSDGWKPEDYVSALLPLASPS

SEQ ID NO: 28 - protein, 24 aa, unidentified
MPRLGQWSRYRRVKTVSSVPVSGP

SEQ ID NO: 29 - protein, 18 aa, unidentified
SALYSFQRKGLANERPFE

SEQ ID NO: 30 - protein, 16 aa, unidentified
QIPHMHSDRKPGPRSL

SEQ ID NO: 31 - protein, 10 aa, unidentified
PSGFFCTSIQ

SEQ ID NO: 32 - protein, 29 aa, unidentified
LLEVHFPLQDLPQTSGSQPVGHTSFGEIE

SEQ ID NO: 33 - protein, 6 aa, unidentified
VSFIIT

SEQ ID NO: 34 - protein, 35 aa, unidentified
QLQSGWWVLDDNLSSHFIQWPHRGLSLINQKPAPL

SEQ ID NO: 35 - protein, 7 aa, unidentified
ISAVGSG

SEQ ID NO: 36 - protein, 26 aa, unidentified
SSGVGESGERVGAAILQQCLLGVMGL

SEQ ID NO: 37 - protein, 31 aa, unidentified
VLLWRERSAYSGFISELQTAWVSGEAGWRGG

SEQ ID NO: 38 - protein, 9 aa, unidentified
QCVGGVARS

SEQ ID NO: 39 - protein, 61 aa, unidentified
FMISLSLWKHLSAGFSSNDTQEPLDSSTGDGGGVERSQASPTPPLCAAFGRVPRLQHYLIN

SEQ ID NO: 40 - protein, 5 aa, unidentified
ASTAT

SEQ ID NO: 41 - protein, 50 aa, unidentified
AQIQKNRVAHVSHSRKSLMAGSQRTMCLPCCPWPAHPECPDSDNGVGTEG

SEQ ID NO: 42 - protein, 7 aa, unidentified
RQCLLYQ

SEQ ID NO: 43 - protein, 82 aa, unidentified
VGPDLLSTASRERAWPMSGLLSSRYLTCILIESLAPDHCDFSPQVSFALQFNDFLRFISLSKICHRPVVLNLWVTPPLGKLN

SEQ ID NO: 44 - protein, 26 aa, synthetic construct
LPLPRSLTRCARSFGCGERYQLTQRR

SEQ ID NO: 45 - protein, 141 aa, synthetic construct
YGYPQNQGITQERTCEQKASKRPGTVKRPRCWRFSIGSAPLTSITKIDAQVRGGETRQDYKDTRRFPLEAPSCALLFRPCRLPDTCPPFSLREAWRFLIAHAVGISVRCRSFAPSWAVCTNPPFSPTAAPYPVTIVLSPTR

SEQ ID NO: 46 - protein, 20 aa, synthetic construct
DTTYRHWQQPLVTGLAERGM

SEQ ID NO: 47 - protein, 6 aa, synthetic construct
AVLQSS

SEQ ID NO: 48 - protein, 19 aa, synthetic construct
SGGLTTATLEEQYLVSALC

SEQ ID NO: 49 - protein, 43 aa, synthetic construct
SQLPSEKELVALDPANKPPLVAVVFLFASSRLRAEKKDLKKIL

SEQ ID NO: 50 - protein, 20 aa, synthetic construct
SFLRGLTLSGTKTHVKGFWS

SEQ ID NO: 51 - protein, 11 aa, synthetic construct
DYQKGSSPRSF

SEQ ID NO: 52 - protein, 23 aa, synthetic construct
IKNEVLNQSKVYMSKLGLTVTNA

SEQ ID NO: 53 - protein, 15 aa, synthetic construct
SVRHLSQRSVYFVHP

SEQ ID NO: 54 - protein, 8 aa, synthetic construct
LPDSPSCR

SEQ ID NO: 55 - protein, 14 aa, synthetic construct
LRYGRAYHLAPVLQ

SEQ ID NO: 56 - protein, 14 aa, synthetic construct
YRETHAHRLQIYQQ

SEQ ID NO: 57 - protein, 30 aa, synthetic construct
TSQPEGPSAEVVLQLYPPPSSLLIVAGKLE

SEQ ID NO: 58 - protein, 85 aa, synthetic construct
VVRQLIVCATLLPLLQASWCHARRLVWLHSAPVPNDQGELHDPPCCAKKRLAPSVLRSLSEVSWPQCYHSWLWQHCIILLLSCHP

SEQ ID NO: 59 - protein, 4 aa, synthetic construct
DAFL

SEQ ID NO: 60 - protein, 35 aa, synthetic construct
LVSTQPSHSENSVCGDRVALARRQYGIIPRHIAEL

SEQ ID NO: 61 - protein, 20 aa, synthetic construct
KCSSLENVLRGENSQGSYRC

SEQ ID NO: 62 - protein, 38 aa, synthetic construct
DPVRCNPLVHPTDLQHLLLSPAFLGEQKQEGKMPQKRE

SEQ ID NO: 63 - protein, 26 aa, synthetic construct
GRHGNVEYSYSSFFNIIEAFIRVIVS

SEQ ID NO: 64 - protein, 13 aa, synthetic construct
ADTYLNVFRKINK

SEQ ID NO: 65 - protein, 16 aa, synthetic construct
GFRAHFPEKCHLNCKR

SEQ ID NO: 66 - protein, 16 aa, synthetic construct
YFVKIRVKFLLNQLIF

SEQ ID NO: 67 - protein, 10 aa, synthetic construct
PIGRNRQNPL

SEQ ID NO: 68 - protein, 42 aa, synthetic construct
IKRIDRDRVECCSSLEQESTIKERGLQRQRAKNRLSGRWPTT

SEQ ID NO: 69 - protein, 13 aa, synthetic construct
TITLIKFFGVEVP

SEQ ID NO: 70 - protein, 6 aa, synthetic construct
STKSEP

SEQ ID NO: 71 - protein, 5 aa, synthetic construct
REPPI

SEQ ID NO: 72 - protein, 22 aa, synthetic construct
SLTGKAGERGEKGREESERSGR

SEQ ID NO: 73 - protein, 18 aa, synthetic construct
GAGKCSGHAARNHHTRRA

SEQ ID NO: 74 - protein, 37 aa, synthetic construct
CAATGRVPFAIQAAQLLGRAIGAGLFAITPAARSLAH

SEQ ID NO: 75 - protein, 32 aa, synthetic construct
GRPGKARASGDLWSPGLSERASAQRGSGQLHH

SEQ ID NO: 76 - protein, 7 aa, synthetic construct
GFLVVND

SEQ ID NO: 77 - protein, 11 aa, synthetic construct
PAMLLIYVAML

SEQ ID NO: 78 - protein, 19 aa, synthetic construct
VPSELHYHLTASEWVVGFG

SEQ ID NO: 79 - protein, 6 aa, synthetic construct
TGLEGI

SEQ ID NO: 80 - protein, 15 aa, synthetic construct
WKLDFQVSIKVTRQV

SEQ ID NO: 81 - protein, 13 aa, synthetic construct
KLGLSRQRRLLRF

SEQ ID NO: 82 - protein, 259 aa, synthetic construct
APIGLTDIHFAFLSTGQRIGSATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 83 - protein, 233 aa, synthetic construct
EFDIKLIDNQPLDYKICERLTGILNYVAPFTLCGYAALMPLYHAIASRMAFIFSSLYKSWLLSLYEELWPVVRQRGVVCTVFADATPTGWGIATTCQLLSGTFAFPLPIATAELIAACLARCWTGARLLGTDNSVVLSGKSSSFPWLLACVATWILRGTSFCYVPSALNPADLPSRGLLPALRPLPRLRLRPQTSRISLWAASPHRYRRPGRPLRADMIRYIDEFGQTTTRMQ

SEQ ID NO: 84 - protein, 45 aa, synthetic construct
KKCFICEICDAIALFVTIISCNKQVNNNNCIHFMFQVQGEMWEVF

SEQ ID NO: 85 - protein, 19 aa, synthetic construct
NLYKCGKIDKDLPRAWLRR

SEQ ID NO: 86 - protein, 7 aa, synthetic construct
VAWRVNH

SEQ ID NO: 87 - protein, 20 aa, synthetic construct
LQGTPSDGVGHSLSARSLAH

SEQ ID NO: 88 - protein, 26 aa, synthetic construct
GRATKGRPTPGLCPGGLSERASAQLH

SEQ ID NO: 89 - protein, 14 aa, synthetic construct
IGQRAGRGGLRIGR

SEQ ID NO: 90 - protein, 6 aa, synthetic construct
FRFLAH

SEQ ID NO: 91 - protein, 28 aa, synthetic construct
LAALGRSAAASGISSLKGGNTVIHRIRG

SEQ ID NO: 92 - protein, 16 aa, synthetic construct
RRKEHVSKRPAKGQEP

SEQ ID NO: 93 - protein, 9 aa, synthetic construct
KGRVAGVFP

SEQ ID NO: 94 - protein, 4 aa, synthetic construct
APPP

SEQ ID NO: 95 - protein, 57 aa, synthetic construct
RASQKSTLKSEVAKPDRTIKIPGVSPWKLPRALSCSDPAAYRIPVRLSPFGKRGAFS

SEQ ID NO: 96 - protein, 29 aa, synthetic construct
VSQFGVGRSLQAGLCARTPRSARPLRLIR

SEQ ID NO: 97 - protein, 16 aa, synthetic construct
VQPGKTRLIATGSSHW

SEQ ID NO: 98 - protein, 16 aa, synthetic construct
QSEVCRRCYRVLEVVA

SEQ ID NO: 99 - protein, 4 aa, synthetic construct
LRLH

SEQ ID NO: 100 - protein, 20 aa, synthetic construct
KNSIWYLRSAEASYLRKKSW

SEQ ID NO: 101 - protein, 10 aa, synthetic construct
LLIRQTNHRW

SEQ ID NO: 102 - protein, 28 aa, synthetic construct
RWFFCLQAADYAQKKRISRRSFDLFYGV

SEQ ID NO: 103 - protein, 32 aa, synthetic construct
RSVERKLTLRDFGHEIIKKDLHLDPFKLKMKF

SEQ ID NO: 104 - protein, 6 aa, synthetic construct
INLKYI

SEQ ID NO: 105 - protein, 4 aa, synthetic construct
VNLV

SEQ ID NO: 106 - protein, 7 aa, synthetic construct
QLPMLNQ

SEQ ID NO: 107 - protein, 75 aa, synthetic construct
GTYLSDLSISFIHSCLTPRRVDNYDTGGLTIWPQCCNDTARPTLTGSRFISNKPASRKGRAQKWSCNFIRLHPVY

SEQ ID NO: 108 - protein, 5 aa, synthetic construct
LLPGS

SEQ ID NO: 109 - protein, 44 aa, synthetic construct
FAQRCCHCYRHRGVTLVVWYGFIQLRFPTIKASYMIPHVVQKSG

SEQ ID NO: 110 - protein, 10 aa, synthetic construct
LLRSSDRCQK

SEQ ID NO: 111 - protein, 14 aa, synthetic construct
VGRSVITHGYGSTA

SEQ ID NO: 112 - protein, 15 aa, synthetic construct
FSYCHAIRKMLFCDW

SEQ ID NO: 113 - protein, 24 aa, synthetic construct
VLNQVILRIVYAATELLLPGVNTG

SEQ ID NO: 114 - protein, 4 aa, synthetic construct
YRAT

SEQ ID NO: 115 - protein, 95 aa, synthetic construct
QNFKSAHHWKTFFGAKTLKDLTAVEIQFDVTHSCTQLIFSIFYFHQRFWVSKNRKAKCRKKGNKGDTEMLNTHTLPFSILLKHLSGLLSHERIHI

SEQ ID NO: 116 - protein, 5 aa, synthetic construct
MYLEK

SEQ ID NO: 117 - protein, 14 aa, synthetic construct
TNRGSAHISPKSAT

SEQ ID NO: 118 - protein, 15 aa, synthetic construct
IVSVNILLKFALNFC

SEQ ID NO: 119 - protein, 7 aa, synthetic construct
ISSFFNQ

SEQ ID NO: 120 - protein, 12 aa, synthetic construct
AEIGKIPYKSKE

SEQ ID NO: 121 - protein, 42 aa, synthetic construct
TEIGLSVVPVWNKSPLLKNVDSNVKGRKTVYQGDGPLREPSP

SEQ ID NO: 122 - protein, 24 aa, synthetic construct
SSFLGSRCRKALNRNPKGSPRFRA

SEQ ID NO: 123 - protein, 114 aa, synthetic construct
RGKPANVARKEGKKAKGAGARALASVAVTLRVTTTPAALNAPLQGASHSPFRLRNCWEGRSVRASSLLRQLRARSLTEAARAKPGRRATFGRPASVSERARREGVANSITRGSL

SEQ ID NO: 124 - protein, 11 aa, synthetic construct
LMINPPCYLST

SEQ ID NO: 125 - protein, 15 aa, synthetic construct
PCSRKSRYPVSFIIT

SEQ ID NO: 126 - protein, 85 aa, synthetic construct
VGPDLLSTASRERAWPMSGLLSSRYLTCILIESLAPDHCDFSPQVSFALQFNDFLRFISLSKICHRPVVLNLWVTPPLGKLNEPV

SEQ ID NO: 127 - protein, 10 aa, synthetic construct
RVYNGSSTSR

SEQ ID NO: 128 - protein, 52 aa, synthetic construct
VSRLQDRFKETNRNWACRDREDSCVSDRHLLVLLTSTLPFSPQGKELDPPPW

SEQ ID NO: 129 - protein, 24 aa, synthetic construct
ARARRTTWPSSRSSCASRCTWRAP

SEQ ID NO: 130 - protein, 23 aa, synthetic construct
TATSSRSRARARAAPTRAPRPPS

SEQ ID NO: 131 - protein, 24 aa, synthetic construct
PRAAPCPSPGTSCPLSSCTAPRPT

SEQ ID NO: 132 - protein, 9 aa, synthetic construct
STPPTSPTT

SEQ ID NO: 133 - protein, 12 aa, synthetic construct
SCPSPRASSGSA

SEQ ID NO: 134 - protein, 7 aa, synthetic construct
TSRTAAW

SEQ ID NO: 135 - protein, 14 aa, synthetic construct
PRTPPCRTASSSTR

SEQ ID NO: 136 - protein, 12 aa, synthetic construct
SCAAPTSPPTAP

SEQ ID NO: 137 - protein, 21 aa, synthetic construct
CRRRPWAGRPPPSGCTPRTAP

SEQ ID NO: 138 - protein, 7 aa, synthetic construct
RARSSRG

SEQ ID NO: 139 - protein, 84 aa, synthetic construct
RTAATTTPRSRPPTRPRSPCSCPAPTTSTSSWTSPPTTRTTPSWNSTSAPRAATPPAAWTSCTSKNSISSLSIINLWITKFVKD

SEQ ID NO: 140 - protein, 17 aa, synthetic construct
LVFLTMLLLLRYVDTLL

SEQ ID NO: 141 - protein, 179 aa, synthetic construct
CLCIMLLLPVWLSFSPPCINPGCCLFMRSCGPLSGNVAWCALCLLTQPPLVGALPPPVSSFPGLSLSPSLLPRRNSSPPALPAAGQGLGCWALTIPWCCRGNHRPFLGCSPVLPPGFCAGRPSATSLRPSIQRTFLPAACCRLCGLFRVFAFALRRVGSPFGPPPRIDTVDPGGRFEQT

SEQ ID NO: 142 - protein, 30 aa, synthetic construct
DTLMSLDKPQLECSEKNALFVKFVMLLLYL

SEQ ID NO: 143 - protein, 50 aa, synthetic construct
AAINKLTTTIAFILCFRFRGRCGRFFKASKTSTNVVKSIRIFLEHGYVDK

SEQ ID NO: 144 - protein, 70 aa, synthetic construct
HGGLIINYKEPLVMELATPSLRARSLTEAGRPKVARRPGFARAASVSERARSCINESANARGEAVCVLGA

SEQ ID NO: 145 - protein, 41 aa, synthetic construct
SASSLTDSLRSVVRLRRAVSAHSKAVIRLSTESGDNAGKNM

SEQ ID NO: 146 - protein, 44 aa, synthetic construct
AKGQQKARNRKKAALLAFFHRLRPPDEHHKNRRSSQRWRNPTGL

SEQ ID NO: 147 - protein, 48 aa, synthetic construct
RYQAFPPGSSLVRSPVPTLPLTGYLSAFLPSGSVALSHSSRCRYLSSV

SEQ ID NO: 148 - protein, 92 aa, synthetic construct
VVRSKLGCVHEPPVQPDRCALSGNYRLESNPVRHDLSPLAAATGNRISRARYVGGATEFLKWWPNYGYTRRTVFGICALLKPVTFGKRVGSS

SEQ ID NO: 149 - protein, 46 aa, synthetic construct
SGKQTTAGSGGFFVCKQQITRRKKGSQEDPLIFSTGSDAQWNENSR

SEQ ID NO: 150 - protein, 13 aa, synthetic construct
GILVMRLSKRIFT

SEQ ID NO: 151 - protein, 4 aa, synthetic construct
ILLN

SEQ ID NO: 152 - protein, 5 aa, synthetic construct
SFKSI

SEQ ID NO: 153 - protein, 4 aa, synthetic construct
SIYE

SEQ ID NO: 154 - protein, 27 aa, synthetic construct
TWSDSYQCLISEAPISAICLFRSSIVA

SEQ ID NO: 155 - protein, 4 aa, synthetic construct
LPVV

SEQ ID NO: 156 - protein, 103 aa, synthetic construct
ITTIREGLPSGPSAAMIPRDPRSPAPDLSAINQPAGRAERRSGPATLSASIQSINCCREARVSSSPVNSLRNVVAIATGIVVSRSSFGMASFSSGSQRSRRVT

SEQ ID NO: 157 - protein, 58 aa, synthetic construct
SPMLCKKAVSSFGPPIVVRSKLAAVLSLMVMAALHNSLTVMPSVRCFSVTGEYSTKSF

SEQ ID NO: 158 - protein, 50 aa, synthetic construct
CMRRPSCSCPASIRDNTAPHSRTLKVLIIGKRSSGRKLSRILPLLRSSSM

SEQ ID NO: 159 - protein, 6 aa, synthetic construct
PTRAPN

SEQ ID NO: 160 - protein, 12 aa, synthetic construct
SSASFTFTSVSG

SEQ ID NO: 161 - protein, 19 aa, synthetic construct
AKTGRQNAAKKGIRATRKC

SEQ ID NO: 162 - protein, 10 aa, synthetic construct
ILILFLFQYY

SEQ ID NO: 163 - protein, 17 aa, synthetic construct
SIYQGYCLMSGYIFECI

SEQ ID NO: 164 - protein, 19 aa, synthetic construct
KNKQIGVPRTFPRKVPPKL

SEQ ID NO: 165 - protein, 5 aa, synthetic construct
ALIFC

SEQ ID NO: 166 - protein, 27 aa, synthetic construct
IFVKSAHFLTNRPKSAKSLINQKNRPR

SEQ ID NO: 167 - protein, 11 aa, synthetic construct
VLFQFGTRVHY

SEQ ID NO: 168 - protein, 37 aa, synthetic construct
RTWTPTSKGEKPSIRAMAHYVNHHPNQVFWGRGAVKH

SEQ ID NO: 169 - protein, 38 aa, synthetic construct
IGTLKGAPDLELDGESRRTWRERKGRKRKERALGRWQV

SEQ ID NO: 170 - protein, 5 aa, synthetic construct
RSRCA

SEQ ID NO: 171 - protein, 63 aa, synthetic construct
PPHPPRLMRRYRARPIRHSGCATVGKGDRCGPLRYYASCALARSLRPPGQSPGVGRPLVARPQ

SEQ ID NO: 172 - protein, 19 aa, synthetic construct
ASERAEREWPTPSLGVPCS

SEQ ID NO: 173 - protein, 19 aa, synthetic construct
LTRHATYLRSHALGRVGTQ

SEQ ID NO: 174 - protein, 49 aa, synthetic construct
MNRSRGYIMEARLPGKYQGYKTGLRRPIETGLVETEKTLAFLIGTYWSY

SEQ ID NO: 175 - protein, 261 aa, synthetic construct
HPLCLSLHRAKNWIRHHGEQGRGGQHGHHQGVHALQGAHGGLRERPRVRDRGRGRGPPLRGHPDRQAEGDQGRPPALRLGHPVPSVHVRLQGLREAPRRHPRLLEAVLPRGLQVGARDELRGRRRGDRDPGLLPAGRRVHLQGEAARHQLPLRRPRNAEEDHGLGGLLRADVPRGRRPEGRDQAEAEAEGRRPLRRRGQDHLQGQEARAAARRLQRQHQAGHHLPQRGLHHRGTVRARRGPPLHRRHGRAVQVRIRYQAYR

SEQ ID NO: 176 - protein, 8 aa, synthetic construct
STSGLQNL

SEQ ID NO: 177 - protein, 6 aa, synthetic construct
KIDWYS

SEQ ID NO: 178 - protein, 32 aa, synthetic construct
LCCSFYAMWIRCFNAFVSCYCFPYGFHFLLLV

SEQ ID NO: 179 - protein, 7 aa, synthetic construct
ILVAVSL

SEQ ID NO: 180 - protein, 17 aa, synthetic construct
GVVARCQATWRGVHCVC

SEQ ID NO: 181 - protein, 47 aa, synthetic construct
RNPHWLGHCHHLSAPFRDFRFPPPYCHGGTHRRLPCPLLDRGSAVGH

SEQ ID NO: 182 - protein, 90 aa, synthetic construct
QFRGVVGEIIVLSLAARLCCHLDSARDVLLLRPFGPQSSGPSFPRPAAGSAASSASSPSPSDESDLPLGRLPASIPSTRAAASSRHDKIH

SEQ ID NO: 183 - protein, 6 aa, synthetic construct
VWTNHN

SEQ ID NO: 184 - protein, 9 aa, synthetic construct
NAVKKMLYL

SEQ ID NO: 185 - protein, 12 aa, synthetic construct
CYCFICNHYKLQ

SEQ ID NO: 186 - protein, 29 aa, synthetic construct
QQQLHSFYVSGSGGDVGGFLKQVKPLQMW

SEQ ID NO: 187 - protein, 4 aa, synthetic construct
SMAT

SEQ ID NO: 188 - protein, 6 aa, synthetic construct
ISSMAG

SEQ ID NO: 189 - protein, 7 aa, synthetic construct
SLTTRNP

SEQ ID NO: 190 - protein, 32 aa, synthetic construct
WSWPLPLCALARSLRPGDQRSPDARALPGRPQ

SEQ ID NO: 191 - protein, 23 aa, synthetic construct
ASERAAALMNRPTRGERRFAYWA

SEQ ID NO: 192 - protein, 286 aa, synthetic construct
WHKILSAGIEAIQRNREDMTAQSGTTYIVVIRSPKGDPGLAAIIGRSGREGAGSKDAIFWGAPLASRLLPGAVKDAEMWDILQQRSALTLLEGTLLKRLTTAMAVPMTTDREDNPIAENLEPEWRDLRTVHDGMNHLFATLEKPGGITTLLLNAATNDSMTIAASCLERVTMGDTLHKETVPSYEVLDNQSYHIRRGLQEQGADIRSLVAGCLLVKFTSMMPFREEPRFSELIKGSNLDLEIYGVRAGLQDEADKVKVLTEPHAFVPLCFAAFFPILAVRFHQISM

SEQ ID NO: 193 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 194 - protein, 4 aa, synthetic construct
RGEI

SEQ ID NO: 195 - protein, 32 aa, synthetic construct; identical to SEQ ID NO: 11

SEQ ID NO: 196 - protein, 89 aa, synthetic construct; identical to SEQ ID NO: 12

SEQ ID NO: 197 - protein, 8 aa, synthetic construct
FLCLCGST

SEQ ID NO: 198 - protein, 22 aa, synthetic construct
VQALVPMTLRSLWIPALGMGVG

SEQ ID NO: 199 - protein, 24 aa, synthetic construct
NVLRPHQPLPCVLPLGESQGFSIT

SEQ ID NO: 200 - protein, 26 aa, synthetic construct
LIRPLLLHRLRFKRTEWPTSAIPGKV

SEQ ID NO: 201 - protein, 27 aa, synthetic construct
WLEARGLCVCLAALGQPILNAQTRTME

SEQ ID NO: 202 - protein, 29 aa, synthetic construct
VQKGKDSVFCTSKWALICSLQLPEKGPGQ

SEQ ID NO: 203 - protein, 8 aa, synthetic construct
VADTSHAF

SEQ ID NO: 204 - protein, 23 aa, synthetic construct
KAWPQITVTLALRFLLHFNSMTS

SEQ ID NO: 205 - protein, 25 aa, synthetic construct
GSFPSPRFATDQWFSTCGSHLLWGN

SEQ ID NO: 206 - protein, 14 aa, synthetic construct
QPFFSFYSVATPWP

SEQ ID NO: 207 - protein, 63 aa, synthetic construct; identical to SEQ ID NO: 24

SEQ ID NO: 208 - protein, 41 aa, synthetic construct; identical to SEQ ID NO: 25

SEQ ID NO: 209 - protein, 41 aa, synthetic construct; identical to SEQ ID NO: 26

SEQ ID NO: 210 - protein, 44 aa, synthetic construct; identical to SEQ ID NO: 27

SEQ ID NO: 211 - protein, 24 aa, synthetic construct
MPRLGQWSRYRRVKTVSSVPVSGP

SEQ ID NO: 212 - protein, 18 aa, synthetic construct
SALYSFQRKGLANERPFE

SEQ ID NO: 213 - protein, 16 aa, synthetic construct
QIPHMHSDRKPGPRSL

SEQ ID NO: 214 - protein, 10 aa, synthetic construct
PSGFFCTSIQ

SEQ ID NO: 215 - protein, 29 aa, synthetic construct
LLEVHFPLQDLPQTSGSQPVGHTSFGEIE

SEQ ID NO: 216 - protein, 35 aa, synthetic construct; identical to SEQ ID NO: 34

SEQ ID NO: 217 - protein, 7 aa, synthetic construct
ISAVGSG

SEQ ID NO: 218 - protein, 26 aa, synthetic construct
SSGVGESGERVGAAILQQCLLGVMGL

SEQ ID NO: 219 - protein, 31 aa, synthetic construct; identical to SEQ ID NO: 37

SEQ ID NO: 220 - protein, 9 aa, synthetic construct
QCVGGVARS

SEQ ID NO: 221 - protein, 61 aa, synthetic construct; identical to SEQ ID NO: 39

SEQ ID NO: 222 - protein, 5 aa, synthetic construct
ASTAT

SEQ ID NO: 223 - protein, 50 aa, synthetic construct; identical to SEQ ID NO: 41

SEQ ID NO: 224 - protein, 7 aa, synthetic construct
RQCLLYQ